(19) United States
(12) Patent Application Publication
(10) Pub. No.: US 2017/0007595 A1
(43) Pub. Date: Jan. 12, 2017

(54) NSD3 INHIBITORS FOR TREATMENT OF CANCERS

(71) Applicants: THE BRIGHAM AND WOMEN'S HOSPITAL, INC., Boston, MA (US); PRESIDENT AND FELLOWS OF HARVARD COLLEGE, Cambridge, MA (US)

(72) Inventors: Christopher A. FRENCH, Newton, MA (US); Peter M. HOWLEY, Wellesley, MA (US); Erica Marlene WALSH MICHEL, Somerville, MA (US); Shaila RAHMAN, Cambridge, MA (US); Simone KUHNLE, Cambridge, MA (US)

(73) Assignees: THE BRIGHAM AND WOMEN'S HOSPITAL, INC., Boston, MA (US); PRESIDENT AND FELLOWS OF HARVARD COLLEGE, Cambridge, MA (US)

(21) Appl. No.: 15/114,020

(22) PCT Filed: Jan. 23, 2015
(86) PCT No.: PCT/US15/12602

§ 371 Date: Jul. 25, 2016

Related U.S. Application Data
(60) Provisional application No. 61/931,334, filed on Jan. 24, 2014.
(60) Provisional application No. 62/003,739, filed on May 28, 2014.

(51) Int. Cl. A61K 31/47; G01N 33/574; C12Q 1/68; A61K 31/5517; A61K 31/4745

(52) U.S. Cl. CPC A61K 31/47; A61K 31/5517; A61K 31/4745; C12Q 1/6886; G01N 33/5748; C12Q 2600/16; C12Q 2600/158; C12Q 2600/156; C12Q 2600/178

(57) ABSTRACT

The present invention relates to the use of NSD3i inhibitors for the treatment of cancer. In particular, the present invention relates to methods, kits and compositions comprising NSD3 inhibitors to treat cancers dependent on NSD3, in particular subjects with NUT midline carcinoma (NMC) and subjects with NSD3/NUT or BRD4/NUT or BRD3/NUT fusion genes, as well as subjects with BRD4-dependent (but NUT-independent cancers). The present invention also relates to methods, kits and compositions comprising BET inhibitors for the treatment of subjects with NSD3/NUT fusion genes. Other aspects of the invention relate to assays and methods to identify an inhibitor of NSD3 which disrupts or decreases the interaction of the NSD3 protein with the ET do main of BRD4.

### CROSS-REFERENCE TO RELATED APPLICATION

This application claims benefit under 35 U.S.C. §119(e) of U.S. Provisional Application No. 61/931,334 filed Jan. 24, 2014 and U.S. Provisional Application No. 62/003,739 filed May 28, 2014, the contents of which are incorporated herein by reference in its entirety.

### GOVERNMENT SUPPORT

This invention was made with Government Support under Grant numbers R01 CA116720, 2R01CA124633-06A1, T32CA009361 and T32HL007627-27 awarded by the National Institutes of Health (NIH). The U.S. Government has certain rights to the invention.

### FIELD OF THE INVENTION

This relates to NSD3 inhibitors, especially a selective NSD3 inhibitor, for use in treating, ameliorating and/or preventing midline carcinoma. Also corresponding methods for treating, preventing or ameliorating midline carcinoma are subject of the present invention. Preferably, NSD3/NUT or BRD or BRD4-dependent cancers midline carcinoma is treated with the NSD3 inhibitors in accordance with the present invention.

### SEQUENCE LISTING

The instant application contains a Sequence Listing which has been submitted electronically in ASCII format and is hereby incorporated by reference in its entirety. Said ASCII copy, created on Jan. 22, 2015, is named 043214-080752-PCT_SL.txt and is 102,716 bytes in size.

### BACKGROUND OF THE INVENTION

Hematopoietic and mesenchymal malignancies are often characterized by translocation-associated fusion oncoproteins that block differentiation, whereas many epithelial cancers are typified by multiple sequential mutations that progress in a multistep pathway to carcinogenesis. One exception of an epithelial carcinoma that is driven by a fusion-oncogene is NUT midline carcinoma (referred to herein as “NMC”). NMC is a highly lethal type of squamous cell carcinoma that has previously been described to occur in young adults and children; see French (2004) J Clin Oncology 22(20), 4135-4139. NMC is defined by chromosomal rearrangement of the NUT gene (also known as NUTM1), which is most commonly fused to the BET family genes BRD4 and BRD3 (1, 2), defined by the presence of dual bromodomains and an extraterminal (ET) domain. BRD-NUT oncoproteins' primary mechanism is to block differentiation and maintain cells in a highly proliferative, poorly differentiated state. This poorly differentiated cancer is far more aggressive than even small cell carcinoma of the lung, with a median survival of 6.7 months (3), and there exist no effective treatment options.

Squamous cell carcinomas are carcinomas that exhibit squamous morphology, and commonly involve organs that are lined by squamous epithelium, including, but not limited to, skin, upper aerodigestive tract, cervix and anus. One type of squamous cell is NUT midline carcinomas, which often, but not exclusively, involves midline organs, including head, neck and medistanum.

NMC is a disease which is genetically defined by rearrangements in the nuclear protein in testis (NUT) gene on chromosome 15q14 most commonly in a balanced translocation with the BRD4 gene or the BRD3 gene. A corresponding rearrangement has first been disclosed in a cell line termed Ty-82 which had been derived from a 22-year old woman with undifferentiated thymic carcinoma; see Kuzume (1992) Int J Cancer 50, 259-264. Later, it has been found that this translocation involving rearrangement in the NUT gene is characteristic for a particularly aggressive form of a midline carcinoma and the term NUT midline carcinoma has been coined; see French (2001) Am J Pathol 159(6), 1987-1992.

NMC as a genetically defined disease does not arise from a specific organ. Most cases occur in the mediastinum and upper aerodigestive tract, but in some cases tumors have arisen in bone, bladder, abdominal retroperitoneum, pancrease and salivary glands; see French (2010), Cancer Genetics and Cytogenetics 203, 16-20 and Ziai (2010) Head and Neck Pathol 4, 163-168.

In about two thirds of NMC cases NUT is fused to BRD4 on chromosome 19; see French (2003) Cancer Res 63, 304-307 and French (2008) Oncogene 27, 2237-2242. French (2008) found that in certain cases NUT may also be fused to BRD3. Further, the inventors previously described the functional role of BRD-NUT fusion proteins using an siRNA assay for silencing expression. It was found that the suppression of expression of such fusion genes results in squamous differentiation and cell cycle arrest and it was concluded that BRD-NUT fusion proteins contribute to carcinogenesis. It has been suggested in the art that NUT rearrangement is a very early, possible tumor-initiating event; see French (2010) J Clin Pathol).

NUT rearrangements are restricted to NMC and can be detected by immunohistochemical testing (e.g. FISH) or by molecular testing like detection of the expression of NUT fusion genes. It is known that NUT can form fusion oncoproteins, such as BRD4-NUT fusion genes, BRD3-NUT fusion genes or fusions of NUT with other uncharacterized genes (termed NUT-variant fusion genes). Also NMC diagnosis via detection of NUT expression with a NUT specific monoclonal antibody has been disclosed in the art; see Haack (2009) Am J Surg Pathol 33(7), 984-991.

Recent excitement in small molecule BET inhibitors arose from the demonstration of the therapeutic targeting of BRD-NUT oncoproteins in NMC in vitro and in pre-clinical models (4). This has led to a clinical trial using the GSK BET inhibitor drug, GSK-525762A, now enrolling NMC and other solid tumors (http://www.clinicaltrials.gov/ct2/show/NCT01587703?term=NMC&rank=1). BET inhibitors are acetyl-histone mimetics that target the acetyl-histone binding pocket of BET protein chromatin-reading bromodomains, such as those of BRD2, 3, 4 and T (4, 5). BET inhibitors induced differentiation and proliferation arrest of NMC, and are a potential form of differentiation therapy in this disease. However, it is not known how interference with chromatin binding leads to inhibition of the blockade of differentiation by BRD-NUT oncoproteins, because the mechanism by which BRD-NUT blocks differentiation is unclear. Evidence suggests that deregulation of MYC expression by BRD-NUT may be key to the blockade of differentiation (6), but it remains to be determined whether BRD-NUT acts directly or indirectly.

Known functional domains of BRD4 that are present in BRD-NUT fusions may give clues to its function. Wild type BRD4 binds to acetylated histones and the positive transcriptional elongation factor, P-TEFb with its bromodomains (7), and is associated with transcriptional activation of target genes (7, 8). Although the function of NUT, an entirely unstructured protein, is unknown, it binds to and activates the histone acetyltransferase (HAT), p300 (9). Both of the bromodomains, and the p300-binding domain are present in BRD-NUT oncoproteins. This has led to the hypothesis that BRD-NUT fusion oncoproteins tether HATs and transcriptional co-factors to chromatin via their bromodomains, leading to a feed-forward process of acetylation and recruitment that results in sequestration of these factors away from pro-differentiation genes to pro-growth genes, such as MYC (2, 9).

The role of the ET domain and its binding proteins has not been investigated in the context of BRD-NUT oncoproteins. Here, the inventors have discovered a novel fusion in a NUT-variant NMC between the methyltransferase protein, NSD3, that has been previously shown to associate with the ET domains of BET proteins (8), and NUT. This discovery demonstrates that NSD3 may be a key component of the BRD-NUT oncogenic complex. Thus, the inventors investigated the oncogenic role of NSD3 in this NUT-variant NMC as well as more typical BRD4-NUT NMCs.

Generally, it is believed that midline carcinoma, especially NMC, is a rare type of cancer; however, most cases of NMC currently go unrecognized due to its lack of characteristic histological features; see French (2010) J Clin Pathol. NMCs are often mistaken for other cancer types such as thymic carcinoma, squamous cell carcinoma of the head and neck, lung carcinoma, Ewing sarcoma, and acute leukemia; (see Schwartz (2011) Cancer Res 71(7), 2686-2696. French (2010) J Clin Pathol.) has proposed to consider any poorly differentiated, monomorphic, midline neoplasm that does not stain for lineage-specific markers for NUT rearrangement testing. Many patients with presently undiagnosed NMC would profit enormously from diagnosis and subsequent effective treatment of NMC.

Unfortunately, an effective therapy of midline carcinoma, such as NMC, is presently not available resulting in a low survival rate (1 survival out of 22 reported cases) and a mean survival of less than 1 year (9.5 months) despite aggressive chemotherapy and radiation treatment, as summarized in Table 1 of French (2010) J Clin Pathol and French (2010), Cancer Genetics and Cytogenetics 203, 16-20. Further, numerous NMC tumors might not be treated at all or treatment might commence late due to a late or absent NMC diagnosis. Although reliable diagnosis of NMC is available, it needs to be improved, and there is also a need in the art for the efficient treatment of midline carcinoma, especially of NMC.

Although there are some existing potential therapies of midline carcinoma, such as NMC, their efficacy against all types of midline carcinoma is inconstant. Schwartz has investigated the mechanism underlying a NMC subtype that is characterized by the expression of the BRD4-NUT fusion gene. Schwartz found that expression of BRD4-NUT is associated with global decrease in histone deacetylation and transcriptional repression. Therefore, Schwartz suggest the use of histone deacetylase inhibitors (HDACi) such as vorinostat and romidesin to treat NMC mediated by the BRD3-NUT oncogene or use of small molecule bromodomain inhibitors (Brdi) to target BRD4-NUT. Schwartz hightlight that the treatments only target BRD4-NUT or BRD3-NUT. The International patent application WO 2010/011700 describes the use of compounds, in particular histone deacetylase inhibitors, that promote increased acetylation of histones for the treatment of a cancer characterized by BRD3-NUT or BRD4-NUT oncoproteins. Also Filippakopoulos (2010) propose use of a BRD4-directed inhibitor termed JQ1 (a thieno-triazolo-1,4-diazepinethe) to treat NMC characterized by BRD4-NUT fusion proteins.

Thus, the technical problem underlying the present invention is that the limited treatments for NMC are only effective, if at all, only on subjects with NMC characterized by BRD4-NUT and BRD3-NUT fusion proteins, resulting in ineffective treatment for NMC characterized by different fusion proteins, and non-BRD-NUT fusion oncoproteins. The technical problem is solved by provision of the embodiments characterized in the claims, and relates to methods and inhibitors for treating, ameliorating and/or preventing midline carcinoma in subjects with NMC characterized by non-BRD-NUT fusion oncoproteins.

### SUMMARY OF THE INVENTION

The present invention is based on the discovery of a new subset of NUT midline carcinoma (NMC) having a NSD3-NUT fusion oncoprotein. Previously known fusion oncoproteins with NUT include fusions with BET (bromodomain and extra-terminal) family proteins BRD4 and BRD3 to form BRD4-NUT fusion genes and BRD3-NUT fusion genes, respectively. It was also known that NUT can form fusion oncoproteins with other uncharacterized genes (termed NUT-variant fusion genes). NMC relates to a genetically defined, very aggressive epithelial cancer that usually arises in the midline of the body and is characterized by a chromosomal rearrangement in the nuclear protein in testis (NUT) gene. In approximately 75% of cases, the coding sequence of NUT on chromosome 15q14 is fused to BRD4 or BRD3, which creates a chimeric gene that encodes the BRD-NUT fusion protein. In the remaining 35% of NMC cases, NUT is fused to an unknown partner gene, usually called NUT-variant.

Herein, the inventors have discovered the identity of an unknown partner gene of the NUT-variant and that NUT can form a fusion oncoprotein with the protein NSD3. Importantly, the inventors have discovered that inhibition of NSD3 decreases proliferation and increases differentiation of NMC cells comprising the NSD3/NUT fusion protein. Surprisingly, it was also discovered that inhibition of NSD3 also decreased the proliferation and increases differentiation of NMC cells comprising BRD/NUT fusion proteins, such as cancer cells with BRD4/NUT or BRD3/NUT fusion oncogenes. Moreover, it was discovered that BET inhibitors (BETi) also decreases proliferation and increases differentiation of NMC cells comprising the NSD3/NUT fusion protein. Accordingly, the present invention relates to a NSD3 inhibitor for use in treating, ameliorating and/or preventing midline carcinoma.

Accordingly, the present invention relates to methods and compositions comprising use of NSD3 inhibitors for the treatment of cancers with NUT fusions, such as, for example NSD3/NUT, BRD4/NUT and BRD3/NUT fusion oncoproteins. In some embodiments, the cancer is NUT midline carcinoma (NMC). In some embodiments, another aspect of the present invention relates to methods and compositions comprising the use of NSD3 inhibitors for the treatment of cancers dependent on NDS3. Such NSD3-dependent cancers include, but are not limited to primary breast carcinomas, pancreatic adenocarcinomas, and acute myeloid leukemia or myelodysplastic syndrome with NU98-NSD3 fusion oncogene.

In alternative embodiments, the present invention relates to methods and compositions comprising use of NSD3 inhibitors (NSD3i) for the treatment of cancers responsive to BET inhibitors (herein also referred to as “BETi”), including but not limited to BRD4-dependent, NUT-independent cancers. Such cancers are well known in the art, and include, for example but not limited to, leukemia, lymphoma, multiple myeloma, neuroblastoma, acute myeloid leukemia (AML), Burkitt lymphomia, Erythroleukemia, Lung adenocarcinoma, B-ALL (B-cell acute lymphoblastic leukemia), Burkitt Lymphoma, APML (Promyelocytic leukemia), Multiple myeloma, Cervical squamous cell carcinoma, Breast carcinoma, Prostate carcinoma and melanoma

In some embodiments, the NSD3 inhibitor is a RNAi inhibitor. In some embodiments, the NSD3 inhibitor is a molecule or entity which disrupts the interaction of NSD3 with a BET protein, such as an entity which inhibits the interaction of NSD3 with BRD4 and/or BRD3. In some embodiments, a NSD3 inhibitor is a large polypeptide that blocks the binding of NSD3 with BRD4 and/or BRD3. In some embodiments, the NSD3 inhibitor is a dominant negative protein, such as, for example, the ET domain of the BRD protein which interacts with NSD3. Accordingly, in some embodiments, the polypeptide inhibitor of NSD3 is comprises a dominant negative protein (e.g., a decoy protein) which binds to NSD3, for example, a protein corresponding to SEQ ID NO: 6, or a fragment of at least 20, or at least 30, or at least 40, or at least 50 or at least 60 or at least 70 or at least 80 consecutive amino acids thereof, or a homologue thereof. In alternative embodiments, the polypeptide inhibitor of NSD3 is comprises a competitive inhibitor, e.g., a soluble fraction of NSD3 which binds to the ET domain of BRD4, for example, a protein corresponding to SEQ ID NO: 4, or a fragment of at least 20, or at least 30, or at least 40, or at least 50 or at least 60 or at least 70 or at least 80, or at least 90, or at least 100 or more than 100 consecutive amino acids thereof, or a homologue thereof.

Another aspect of the present invention relates to use of BET inhibitors (BETi) for cancers comprising overexpressed NDS3, or cancers comprising the NDS3/NUT fusion oncoprotein. In some embodiments, the cancer is NUT midline carcinoma (NMC), pancreatic adenocarcinoma, breast adenocarcinoma and acute myeloid leukemia. Such BET inhibitors are well known in the art and include, for example, but are not limited to JQ1, disclosed in WO/2009/084693 and GSK-525762A (also known as I-BET762, Wynce et al., Oncotarget. 2013; 4(12): 2419-2429 Inhibition of BET bromodomain proteins as a therapeutic approach in prostate cancer), LY294002 (Dittmann et al., “The Commonly Used PI3-Kinase Probe LY294002 is an Inhibitor of BET Bromodomains”. ACS Chemical Biology: 2013, 131210150813004.). BET inhibitors are also disclosed in US Application 2012/0208800 and International Applications WO201105484 and WO2006/032470 (SmithKline Beecham Corporation), which are each incorporated herein in their entirety. Such compounds can be prepared by methods described therein.

In a further embodiment a BET inhibitor is a compound that is generically or specifically disclosed in PCT publication WO2009/084693 (Mitsubishi Tanabe), which is incorporated herein in its entirety by reference. Such compounds can be prepared by methods described therein. In a further embodiment a BET inhibitor is 1-[2-(1/-/-benzimidazol-2-ylthio)ethyl]-1,3-dihydro-3-methyl-2H-benzinidazole-2-thione as described in Japanese patent application JP2008-15631 1, which is incorporated herein in its entirety. It will be appreciated that a BET inhibitor used in the present invention may be in the form of a pharmaceutically acceptable salt, solvate (e.g. a hydrate) or prodrug or any other derivative of such a compound which upon administration to the recipient is capable of providing (directly or indirectly) the BET inhibitor of the invention, or an active metabolite or residue thereof. Suitable pharmaceutically acceptable salts can include acid or base addition salts. For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent. Suitable prodrugs are recognizable to those skilled in the art, without undue experimentation.

Another aspect of the present invention relates to use of NSD3 inhibitors (NSD3i) for treatment of diseases and disorders in which a BET inhibitor is indicated or useful. Such diseases include, but are not limited to, cancers, autoimmune diseases, systemic inflammatory response syndrome, such as sepsis, burns, pancreatitis, major trauma, hemorrhage and ischemia. In such an embodiment, a BET inhibitor can be administered at the point of diagnosis to reduce the incidence of: SIRS, the onset of shock, multi-organ dysfunction syndrome, which includes the onset of acute lung injury, ARDS, acute renal, hepatic, cardiac and gastro-intestinal injury and mortality. In another embodiment a BET inhibitor would be administered prior to surgical or other procedures associated with a high risk of sepsis, hemorrhage, extensive tissue damage, SIRS or MODS (multiple organ dysfunction syndrome). In some embodiments a BET inhibitor is indicated for the treatment of sepsis, sepsis syndrome, septic shock or endotoxaemia. In another embodiment, a BET inhibitor is indicated for the treatment of acute or chronic pancreatitis. In another embodiment a BET inhibitor is indicated for the treatment of burns.

Another aspect of the present invention relates to use of NSD3 as a biomarker for use as a companion diagnostic to identify subjects who are responsive to BETi. Another aspect of the present invention relates to use of NSD3 as a biomarker to identify subjects with NSD3/NUT fusion oncoprotein and having NMC.

Another aspect relates to a high throughput screen (HTS) to identify compounds which disrupt the interaction between NSD3 and a BET protein, e.g., BRD4 or BRD3. In some embodiments, an exemplary assay can be performed by one of ordinary skill in the art, for example, such an assay can comprise at least the N-terminal region of NSD3 which binds to the BET protein, and at least a portion of the BET protein which interacts with the NSD3 protein, and an agent which produces a florescent signal when NSD3 complexes with either BRD3 or BRD4. A NSD3 inhibitor which disrupts the interaction between NSD3 and a BET protein, e.g., BRD4 or BRD3 can be identified by identifying a decrease in the fluorescent signal on addition of the agent to the NSD3-BRD3/4 complex.

These and other aspects of this invention will be apparent upon reference to the following detailed description. To that end, certain patent and other documents are cited herein to more specifically set forth various aspects of this invention. Each of these documents are hereby incorporated by reference in their entirety.

### BRIEF DESCRIPTION OF FIGURES

FIGS. 1A-1I show a novel NSD3-NUT fusion in NUT midline carcinoma. FIG. 1A shows the histology of the NMC from which the 1221 cell line was derived reveals a very poorly differentiated tumor (400× magnification). FIG. 1B shows immunohistochemistry of the tumor using the anti-NUT monoclonal antibody, C52 (400× magnification). FIG. 1C shows that RNA-sequencing reads spanning the junction of NSD3 and NUT (SEQ ID NOS 72-113, respectively, in order of appearance). FIG. 1D shows an immunoblot of three NMC cell lines and 293T control cells stained with AX.1 polyclonal antibody to NUT. FIG. 1E shows an immunoblot of the 1221 cell line 48 h following transfection with control (CTRL), NSD3, and NUT siRNAs stained with the AX.1 antibody to NUT. (F) NSD3-NUT dual color bring-together fluorescent in situ hybridization assay (1000× magnification) using BAC probes telomeric (3′) to NUT (green), and BAC probes centromeric (5′) to NSD3 (red) as depicted in the chromosomes 8 and 15 ideograms. Yellow arrows indicate NSD3-NUT fusions. FIG. 1G shows gel electrophoresis of PCR of TC-797 and 1221 cell lines with (+) and without (−) reverse transcriptase reaction. FIG. 1H is a schematic drawing of the NSD3-NUT predicted encoded protein in comparison with NSD3, NUT, and BRD4-NUT. Abbreviations: PWWP, Pro-Trp-Trp-Pro motif (SEQ ID NO: 13); PHD, PHD finger (Plant Homeo Domain); SET, Su(var)3-9, Enhancer-of-zeste and Trithorax domain; C/H rich, Cys-His-rich region; NLS, nuclear localization sequence; NES, nuclear export signal sequence; Bromo, bromodomain; ET, extra-terminal domain. Arrows indicated breakpoints. FIG. 1I shows NSD3 dual color split-apart fluorescent in situ hybridization assay using BAC probes flanking NSD3, as depicted in the chromosome 8 ideogram, depicted in three NMCs designated cases 1-3. All photomicrographs are identical magnification (1000×).

FIGS. 2A-2D show NSD3-NUT is required for the blockade of differentiation and maintenance of proliferation in 1221 NMC cells. FIG. 2A shows a high throughput 384 well plate immunofluorescent assay of keratin using the DAPI nuclear counterstain in 1221 cells 72 h following transfection with control, NUT, or NSD3 siRNAs. Representative photos are identical magnification (400×). FIG. 2B shows that using the high-throughput assay in (A) quantitative analysis of keratin intensity was compared in 1221 cells 72 h following transfection with control, NUT, NSD3-5′ (targets both NSD3-NUT and NSD3-full length), and NSD3-3′ (targets the NSD3 portion not included in NSD3-NUT). Two different siRNAs were used for each gene or region targeted. Representative results from one of three biological replicates, each performed in triplicate, are shown. Error bars indicate the mean±SD of the triplicate wells. FIG. 2C shows that proliferation assay (Ki-67 fraction) using the high-throughput assay comparing 1221 cells transfected with control, NUT, and NSD3 siRNAs. Shown are averages of three biological replicates, each performed in triplicate. Error bars indicate the mean±SD of the three biological replicates. FIG. 2D shows the cell number determined using the high-throughput assay comparing 1221 cells transfected with control, NUT, and NSD3 siRNAs. Shown are averages of three biological replicates, each performed in triplicate. Error bars indicate the mean±SD of the three biological replicates. * p<0.01; **p<0.05.

FIGS. 3A-3F show wild type NSD3 is required for the blockade of differentiation in BRD4-NUT-expressing NMC cells. FIG. 3A shows immunoblots of BRD4-NUT-positive NMC cell lines TC-797, PER-403, and 8645 120 h following transfection with control and NSD3 siRNAs stained with the terminal squamous differentiation marker, involucrin, using GAPDH as loading control. FIG. 3B shows representative photomicrographs of TC-797s 120 h following transfection with either control, or NSD3 siRNAs stained either with hematoxylin and eosin (H&E) for morphology, or involucrin immunohistochemistry. All photos are identical magnification (400×). FIG. 3C shows quantitative RT-PCR results of NSD3 levels 24 h following transfection of control or NSD3 siRNAs. Primers were either 5′ of the breakpoint (NSD3-5′ primers), or 3′ of the breakpoint (NSD3-3′ primers) with NUT. Results are of a single biological replicate performed in triplicate. Error bars indicate the mean±SD of the triplicate wells. FIG. 3D shows results of a proliferation assay (Ki-67 fraction) comparing BRD4-NUT-positive TC-797, 8645, and PER-403 NMC cells transfected with control and NSD3-6 siRNAs. Three hundred cells were counted per cell block. FIG. 3E shows 797TRex cells induced to express FLAG-tagged NLS-ET domain of BRD4 for 120 h Immunoblot was stained with anti-involucrin (Inv), anti-FLAG, or anti-GAPDH (left). Cell block preparations were H&E stained, or subjected to involucrin immunohistochemistry (right). All photos are identical magnification (400×). FIG. 3F shows the results from a cell viability assay (CellTiter-Glo) of 797TRex, 293TRex, and U2OSTRex cells induced to express FLAG-tagged NLS-ET domain for 120 h. Results are the average of three biological replicates, each performed in quadruplet and normalized to the negative control (ethanol vehicle control) for each cell line. Error bars indicate the mean±SD of the three biological replicates Immunoblot demonstrating NLS-FLAG-ET expression was stained with anti-FLAG, or anti-GAPDH (right).

FIGS. 4A-4D show the N-terminus of NSD3 associates with BRD4 and BRD4-NUT. FIG. 4A shows immunofluorescence microscopy of 797TRex cells induced to express the HA-tagged portion of NSD3 included in NSD3-NUT (NSD3Tr) for 24 h stained with anti-NUT monoclonal antibody (red), and anti-HA monoclonal antibody (green). FIG. 4B shows immunoblot of anti-HA immunoprecipitations of tet-repressor-positive C33A cell (C33A-6TR) lysates following induction of expression of HA-tagged NSD3 variants, HA-NSD3 (full length), HA-NSD3-NUT and HA-NSD3-tr (NSD3 portion of the NSD3-NUT fusion protein). Indicated proteins were detected using anti-HA, and anti-Brd4 antibodies. The smaller bands are degraded protein. FIG. 4C shows an immunoblot of anti-HA immunoprecipitations of C33A-6TR lysates following induction of expression of HA-tagged NUT, BRD4, and BRD4-NUT constructs stained with anti-HA, -NSD3, -p300, and -actin antibodies. To identify the NSD3-specific bands, lysates from TC-797s subjected to siRNA knockdown of NSD3 are shown. FIG. 4D shows an immunoblot of 797TRex lystes 120 h following induction of expression of BioTAP-tagged NLS-fusion construct of NSD3Tr stained with anti-involucrin, -PAP (recognizes the protein A moiety of the BioTAP tag), and -GAPDH antibodies.

FIGS. 5A-5C show BRD4-NUT foci are dependent on NSD3. FIG. 5A shows immunofluorescence microscopy of TC-797 cells 24 h following transfection with control or NSD3-6 siRNAs stained with monoclonal antibody to NUT. All photos are identical magnification (1000×). FIG. 5B shows the quantitation of BRD4-NUT foci was performed in triplicate and the averages of the three experiments. Error bars indicate the mean±SD of triplicate experiments. *p<0.005. FIG. 5C shows an immunoblot of TC-797 lysates 24 h following transfection with control, NSD3-6, or NUT siRNAs stained with anti-NUT polyclonal antibody, AX.1.

FIGS. 6A-6C show NSD3-NUT can replace the function of BRD4-NUT to lock differentiation. FIG. 6A shows H&E and anti-involucrin immunohistochemistry micrographs of 797TRex cells with tetracycline (ON), or treated with vehicle (OFF) to express NSD3-NUT 120 h following transfection with either control or NUT 3′UTR siRNA. All photos are identical magnification (400×). FIG. 6B shows immunoblots using lysates corresponding to the experiment in FIG. 6A, were performed for the differentiation marker, involucrin, NSD3-NUT, and BRD4-NUT using antibodies to NUT. FIG. 6C shows the quantification of immunohistochemical Ki-67 proliferation fraction of 797TRex cells induced to express NSD3-NUT 120 h following transfection with either control or NUT 3′UTR siRNA as in FIG. 6A. Results are the average of three biological replicates performed using the 384-well high throughput assay as in FIG. 2A, each performed in triplicate. Error bars indicate the mean±SD of the three biological replicates. *p<0.0001.

FIGS. 7A-7D shows BRD4 inhibition arrests proliferation and induces differentiation of NSD3-NUT-expressing NMC cells. FIG. 7A shows use of the 384-well plate high-throughput assay exhibited in FIG. 2A, quantitative analysis of keratin intensity was compared in 1221 cells 72 h following transfection with control versus BRD4 siRNAs. Representative results from one of three biological replicates, each performed in triplicate, are shown. Error bars indicate the mean±SD of triplicate wells. FIG. 7B shows that using the high-throughput assay, quantitative analysis of keratin intensity was compared in 1221 cells 72 h following treatment with a dose range of JQ1 versus DMSO vehicle control. Results are the average of three biological replicates performed using the 384-well high throughput assay, each performed in triplicate. Error bars indicate the mean±SD of the three biological replicates. * p<0.01. FIG. 7C shows representative immunofluorescence microscopy of 1221 cells treated as in (7B), with vehicle control or 500 nM JQ1 for 72 h. All photos are identical magnification (400×). FIG. 7D shows the cell number using the high-throughput assay comparing 1221 cells 72 h following treatment with increasing concentrations of JQ1 versus DMSO vehicle control. Results are the average of three biological replicates, each performed in triplicate. Error bars indicate the mean±SD of the three biological replicates. * p<0.01.

FIG. 8 shows karyotype of the 1221 NSD3-NUT-positive cell line demonstrating 45,XX,der(3)t(3;6)(q24;p12),del(6p12)der(88)t(8;15)(p12;q15),der99)t(9;10)(p13;q11.2),-15.

FIG. 9 shows NUT is translocated to the NSD3 locus in 1221 cell line. FISH on metaphase spread of 1221 cell line using centromeric (green) and telomeric (red) probes flanking the NUT gene, reveal loss of derivative chromosome 15, and translocation of the telomeric NUT probe proximal to the NSD3 locus on chromosome 8.

FIG. 10 shows wild type NSD3 is required for the blockade of differentiation in BRD4-NUT-expressing NMC cells. Representative photomicrographs of BRD4-NUT-expressing NMC cell lines, 8645 and PER-43 following transfection with either control, or NSD3 siRNAs stained with hematoxylin and eosin for morphology. All photos are identical magnification (400×).

FIGS. 11A-11B shows that among several ET-interacting proteins, only HSD3 is required for the blockade of differentiation in NMC. FIG. 11A shows quantitative RT-PCR of NSD3, JMJD6, GLTSCR1, and ATAD5 levels 24 h following transfection of siRNAs. Results are of a single biological replicate performed in triplicate. Error bars indicate the mean+SD of the triplicate wells. Efficient (approx. 85%) knockdown of CHD4 protein levels using the siRNAs shown in (11B) has been previously demonstrated (ref 8, FIG. 5A). FIG. 11B shows immunoblots of TC-797 lysates 120 h following transfection with siRNAs stained with involucrin, using histone H3 as loading control.

### DETAILED DESCRIPTION

As discussed herein, the present invention provides methods and compositions comprising use of NSD3 inhibitors for the treatment of cancers with NUT fusions, such as, for example NSD3/NUT, BRD4/NUT and BRD3/NUT fusion oncoproteins. In some embodiments, the cancer is NUT midline carcinoma (NMC). In some embodiments, another aspect of the present invention relates to methods and compositions comprising the use of NSD3 inhibitors for the treatment of cancers dependent on NDS3. Such cancers include, but are not limited to primary breast carcinomas, pancreatic adenocarcinomas, acute myeloid leukemia and myelodysplastic syndrome etc.

In alternative embodiments, the present invention relates to methods and compositions comprising use of NSD3 inhibitors (NSD3i) for the treatment of cancers responsive to BET inhibitors (herein also referred to as “BETi”), including but not limited to BRD4-dependent, NUT-independent cancers. Such cancers are well known in the art, and include, for example but not limited to, leukemia, lymphoma, multiple myeloma, neuroblastoma, acute myeloid leukemia (AML), Burkitt lymphomia, Erythroleukemia, Lung adenocarcinoma, B-ALL (B-cell acute lymphoblastic leukemia), Burkitt Lymphoma, APML (Promyelocytic leukemia), Multiple myeloma, Cervical squamous cell carcinoma, Breast carcinoma, Prostate carcinoma and melanoma.

In some embodiments, the NSD3 inhibitor is a RNAi inhibitor. In some embodiments, the NSD3 inhibitor is a molecule or entity which disrupts the interaction of NSD3 with a BET protein, such as an entity which inhibits the interaction of NSD3 with BRD4 and/or BRD3. In some embodiments, a NSD3 inhibitor is a large polypeptide that blocks the binding of NSD3 with BRD4 and/or BRD3. In some embodiments, the NSD3 inhibitor is a dominant negative protein, such as, for example, the ET domain of the BRD protein which interacts with NSD3.

Another aspect of the present invention relates to use of BET inhibitors (BETi) for cancers comprising overexpressed NDS3, or cancers comprising the NDS3/NUT fusion oncoprotein. In some embodiments, the cancer is NUT midline carcinoma (NMC). Such BET inhibitors are well known in the art and include, for example, but are not limited to JQ1, disclosed in WO/2009/084693, which is incorporated herein in its entirety by reference, and GSK-525762A (also known as I-BET762).

Another aspect of the present invention relates to use of NSD3 inhibitors (NSD3i) for treatment of diseases and disorders in which a BET inhibitor is indicated or useful.

Another aspect of the present invention relates to use of NSD3 as a biomarker for use as a companion diagnostic to identify subjects who are responsive to BETi. Another aspect of the present invention relates to use of NSD3 as a biomarker to identify subjects with NSD3/NUT fusion oncoprotein and having NMC.

Another aspect relates to a high throughput screen (HTS) to identify compounds which disrupt the interaction between NSD3 and a BET protein, e.g., BRD4 or BRD3. In some embodiments, an exemplary assay can be performed by one of ordinary skill in the art, for example, such an assay can comprise at least the N-terminal region of NSD3 which binds to the BET protein, and at least a portion of the BET protein which interacts with the NSD3 protein, and an agent which produces a florescent signal when NSD3 complexes with either BRD3 or BRD4. A NSD3 inhibitor which disrupts the interaction between NSD3 and a BET protein, e.g., BRD4 or BRD3 can be identified by identifying a decrease in the fluorescent signal on addition of the agent to the NSD3-BRD3/4 complex.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “NUT midline carcinoma” or “NMC” refers to a genetically defined, very aggressive epithelial cancer that usually arises in the midline of the body and is characterized by a chromosomal rearrangement in the nuclear protein in testis (NUT) gene. In approximately 75% of cases, the coding sequence of NUT on chromosome 15q14 is fused to BRD4 or BRD3, which creates a chimeric gene that encodes the BRD-NUT fusion protein. The remaining cases, the fusion of NUT is to an unknown partner gene, usually called NUT-variant, or as disclosed herein, NSD2-NUT fusion protein. The symptoms of NMC are similar to other forms of cancer and dependent on the stage; while generalized symptoms (weight loss and fatigue) may be seen, site specific symptoms are also present. If the tumor involves the head and neck region (in about 35%), then pain, a mass, obstructive symptoms, among others, may be experienced. NUT midline carcinomas are not specific to any tissue type or organ; common sites include the head, neck and mediastinum. The median age at diagnosis is 17 years, but older patients may be affected.

The terms “NSD3 inhibitor” or “NSD3i” are used interchangeably herein, generally refers to an agent or molecule that inhibits the activity or expression of NSD3. NSD3 inhibitors can be of synthetic or biological origins. They can be organic, or inorganic molecules, or peptides, antibodies or antisense RNA that inhibit NSD3 Inhibitors of NSD3 of the invention are chemical entities or molecules that can inhibit expression of NSD3 and/or biological activity of NSD3 and/or the interaction of NSD3 with BET proteins such as inhibit NSD3 interaction with BRD4 and/or BRD3. NSD3i include, for example, RNAi agents, antisense nucleic acids, dominant negative proteins (decoy molecules), large polypeptides, or modified RNA (modRNA) which express decoy proteins, compounds of trifluroperazine (TFP), flurophenazine, perphenazine, and naphthalenesulfonamides, and enantiomers, prodrugs, derivatives and pharmaceutically acceptable salts thereof, which are discussed further in the section.

As used herein, the term “BET inhibitor” or “BETi” denotes a compound which inhibits the binding of a bromodomain with its cognate acetylated proteins. In one embodiment the BET inhibitor is a compound which inhibits the binding of a BET protein to acetylated lysine residues. In a further embodiment the BET inhibitor is a compound which inhibits the binding of a BET protein to acetylated lysine residues on histones, particularly histones H3 and H4. In a particular embodiment the BET inhibitor is a compound that inhibits the binding of BET family bromodomains to acetylated lysine residues (hereafter referred to as a “BET family bromodomain inhibitor”). In one embodiment the BET family bromodomain is BRD2, BRD3 or BRD4, in particular BRD2 or BRD3. A BET family bromodomain inhibitor is a compound which has a plC50≧5.0 of at least in one or more of the binding assays described herein, or described in International Patent Application WO2013/026874, which is incorporated herein in its entirety by reference.

As used herein, “gene silencing” or “gene silenced” in reference to an activity of an RNAi molecule, for example a siRNA or miRNA refers to a decrease in the mRNA level in a cell for a target gene (e.g. NSD3 gene) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein to inhibit the NSD3 gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “gene product(s)” as used herein refers to include RNA transcribed from a gene, or a polypeptide encoded by a gene or translated from RNA.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “lower”, “reduced”, “reduction” or “decrease”, “down-regulate” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. When “decrease” or “inhibition” is used in the context of the level of expression or activity of a gene or a protein, e.g. NSD3, it refers to a reduction in protein or nucleic acid level or activity in a cell, a cell extract, or a cell supernatant. For example, such a decrease may be due to reduced RNA stability, transcription, or translation, increased protein degradation, or RNA interference. In some embodiments, a NSD3 inhibitor which is a small-molecule as disclosed herein can decrease the activity or expression of NSD3. Preferably, this decrease is at least about 5%, at least about 10%, at least about 25%, at least about 50%, at least about 75%, at least about 80%, or even at least about 90% of the level of expression or activity under control conditions. The term “level” as used herein in reference to NSD3 refers to expression or activity of NSD3.

The term “fragment” of a peptide, polypeptide or molecule as used herein refers to any contiguous polypeptide subset of the molecule. The term “protein fragment” as used herein includes both synthetic and naturally-occurring amino acid sequences derivable from the naturally occurring amino acid sequence. Accordingly, a “fragment” of a molecule, is meant to refer to any polypeptide subset of the molecule.

The term “non-functional” as used herein in conjunction with a “non-functional mimetic of NSD3” refers to a polypeptide which comprises at least a portion of the NSD3 protein of SEQ ID NO:4 or SEQ ID NO:2 but does not retain the natural function of NSD3 of binding and interacting with BRD4 and triggering BRD4 signaling. In some embodiments, a non-functional mimetic of NSD3 comprises a mimetic of the portion of NSD3 which binds to BRD4 (e.g., SEQ ID NO:4, or a fragment thereof) which has an ectopic mutation or amino acid change that allows it to still binds to BRD4 and other ligands of NSD3 but does not allow NSD3-mediated intracellular signaling, including activation of BRD4.

The term “linker” refers to any means to join two or more entities. For example a non-functional mimetic of NSD3 (e.g., SEQ ID NO: 4 or a fragment thereof comprising at least one ectopic mutation) or a mimetic of the ET domain of BRD4 (e.g., SEQ ID NO: 6 or a fragment thereof, and optionally comprising at least one ectopic mutation) as disclosed herein can be joined with a first fusion partner (e.g. Fc). A linker can be a covalent linker or a non-covalent linker. Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling. It will be appreciated that modification which do not significantly decrease the function of a non-functional NSD3 mimetic as disclosed herein or the first fusion partner (e.g. Fc) are preferred.

The terms “subject” and “individual” are used interchangeably herein, and refer to an animal, for example a human, to whom treatment for cancer or a proliferative disorder, including therapeutic treatment or prophylactic treatment, with a pharmaceutical composition comprising a NSD3i or BETi as disclosed herein can be administered. The term “subject” as used herein includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term “non-human animals” and “non-human mammals” are used interchangeably herein and includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. In one embodiment, the subject is human. In another embodiment, the subject is an experimental animal or animal substitute as a disease model, including transgenic non-human animal species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposion, affection.

The term “cancer” and “malignancy” are used interchangeably herein, refers to diseases that are characterized by uncontrolled, abnormal growth of cells. In some embodiments, the term cancer encompasses cancer cells which have spread locally or through the bloodstream and lymphatic system to other parts of the body, referred to herein as “metastatic cancer”. The term is also intended to include any disease of an organ or tissue in mammals characterized by poorly controlled or uncontrolled multiplication of normal or abnormal cells in that tissue and its effect on the body as a whole. Cancer diseases within the scope of the definition comprise benign neoplasms, dysplasia's, hyperplasias as well as neoplasms showing metastatic growth or any other transformations like e.g. leukoplakias which often precede a breakout of cancer.

As used herein, the term “tumor” refers to a mass of transformed cells that are characterized, at least in part, by containing angiogenic vasculature. The transformed cells are characterized by neoplastic uncontrolled cell multiplication which is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease the natural course of which is fatal. Cancer cells exhibit the properties of invasion and metastasis and are highly anaplastic.

As used herein, the terms “metastases” or “metastatic tumor” “metastatic cancer” are used interchangeably herein and refer to a secondary tumor that grows separately elsewhere in the body from the primary tumor and has arisen from detached cancer cells from the primary tumor which have been transported to a separate location, and where the primary tumor is a solid tumor. The primary tumor, as used herein, refers to a tumor that originated in the location or organ in which it is present and did not metastasize to that location from another location. As used herein, a “malignant tumor” or “metastatic cancer” is one having the properties of invasion and metastasis and showing a high degree of anaplasia. Anaplasia is the reversion of cells to an immature or a less differentiated form, and it occurs in most malignant tumors.

The term “therapy resistant cancer” as used herein refers to a cancer present in a subject which is resistant to, or refractory to at least two different anti-cancer agents such as chemotherapy agents, which means, typically a subject has been treated with at least two different anti-cancer agents that did not provide effective treatment as that term is defined herein.

As used herein, the terms “treat” or “treatment” or “treating” refers to therapeutic treatment, wherein the object is to prevent or slow the development of the disease, such as slow down the development of a tumor, the spread of cancer, or reducing at least one effect or symptom of a condition, disease or disorder associated with inappropriate proliferation or a cell mass, for example cancer. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cancer, as well as those likely to develop secondary tumors due to metastasis.

The term “prophylactic treatment” refers to the prevention of the development of cancer in a subject when the subject is at a high risk of developing cancer, such as, for example, a predisposition to cancer where the subject has a genetic mutation or polymorphism known to increase occurrence of a cancer, or a family history of cancer. In some embodiments, prophylactic treatment is used in a subject who has been successfully therapeutically treated for cancer and where the cancer has been eliminated or the subject has gone into remission, and is administered prophylactic treatment with comprising a NSD3i or BETi to prevent a cancer relapse.

The term “effective amount” as used herein refers to the amount of therapeutic agent-comprising a NSD3i or BETi as disclosed herein, to alleviate at least one or more symptom of the disease or disorder, and relates to a sufficient amount of pharmacological composition to provide the desired effect, e.g., to stop or reduce or lessen at least one symptom of the disease or disorder or cancer. The phrase “therapeutically effective amount” as used herein, e.g., a pharmaceutical composition comprising at least one NSD3i or BETi as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylactically significant reduction in a symptom or clinical marker associated with a cancer or a cancer-mediated condition.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated.

With reference to the treatment of a subject with a cancer with a pharmaceutical composition comprising at least one NSD3i or BETi as disclosed herein, the term “therapeutically effective amount” refers to the amount that is safe and sufficient to prevent or delay the development and further growth of a tumor or the spread of metastases in cancer patients. The amount can thus cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor metastases. The effective amount for the treatment of cancer depends on the tumor to be treated, the severity of the tumor, the drug resistance level of the tumor, the species being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of cancer and tumor, for example treatment of a rodent with a cancer, and any treatment or administration of the compositions or formulations that leads to a decrease of at least one symptom of the cancer, for example a reduction in the size of the tumor or a slowing or cessation of the rate of growth of the tumor indicates effective treatment. In embodiments where the compositions are used for the treatment of cancer, the efficacy of the composition can be judged using an experimental animal model of cancer, e.g., wild-type mice or rats, or preferably, transplantation of tumor cells. When using an experimental animal model, efficacy of treatment is evidenced when a reduction in a symptom of the cancer, for example a reduction in the size of the tumor or a slowing or cessation of the rate of growth of the tumor occurs earlier in treated, versus untreated animals. By “earlier” is meant that a decrease, for example in the size of the tumor occurs at least 5% earlier, but preferably more, e.g., one day earlier, two days earlier, 3 days earlier, or more.

As used herein, the term “treating” when used in reference to a cancer treatment is used to refer to the reduction of a symptom and/or a biochemical marker of cancer, for example a reduction in at least one biochemical marker of cancer by at least about 10% would be considered an effective treatment. Examples of such biochemical markers of cancer include CD44, telomerase, TGF-α, TGF-β, erbB-2, erbB-3, MUC1, MUC2, CK20, PSA, CA125 and FOBT. A reduction in the rate of proliferation of the cancer cells by at least about 10% would also be considered effective treatment by the methods as disclosed herein. As alternative examples, a reduction in a symptom of cancer, for example, a slowing of the rate of growth of the cancer by at least about 10% or a cessation of the increase in tumor size, or a reduction in the size of a tumor by at least about 10% or a reduction in the tumor spread (i.e. tumor metastasis) by at least about 10% would also be considered as affective treatments by the methods as disclosed herein. In some embodiments, it is preferred, but not required that the therapeutic agent actually kill the tumor.

As used herein, the terms “administering,” and “introducing” are used interchangeably herein and refer to the placement of the pharmaceutical compositions of the present invention comprising a NSD3i or BETi as disclosed herein into a subject by a method or route which results in at least partial localization of the NSD3i or BETi at a desired site. The compounds of the present invention can be administered by any appropriate route which results in an effective treatment in the subject.

The phrases “parenteral administration” and “administered parentally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. The phrases “systemic administration,” “administered systemically”, “peripheral administration” and “administered peripherally” as used herein mean the administration of the pharmaceutical compositions of the present invention comprising NSD3i or BETi and optionally other agents or material other than directly into the central nervous system, such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The articles “a” and “an” are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### NSD3 Inhibitors (NSD3i)

As mentioned above, the present invention is directed to NSD3 inhibitors for the treatment of diseases and disorders comprising NSD3/NUT fusion oncoproteins or BRD4/NUT or BRD3/NUT fusion oncoproteins, or diseases in which treatment with a BET inhibitor would be beneficial.

Accordingly, the present invention relates in part to methods and compositions to inhibit NSD3. In some embodiments, NSD3 inhibitors as disclosed herein can be used to inhibit the cellular NSD3 activity. In some embodiments, NSD3 inhibitors as disclosed herein can decrease expression (level) of NSD3. In some embodiments, the NSD3 inhibitors inhibit the interaction of NSD3 with BET proteins, such as BRD4 and BRD3.

The ability of a compound to inhibit NSD3 can be assessed by measuring a decrease in activity of NSD3 as compared to the activity of NSD3 in the absence of a NSD3 inhibitor. In some embodiments, the ability of a compound to inhibit NSD3 can be assessed by measuring a decrease in the biological activity (e.g., protein activity), e.g., NSD3-dependent enzyme activity, or decrease in NSD3 expression as compared to the level of NSD3 activity and/or expression in the absence of NSD3 inhibitors.

### RNAi Inhibitors of NSD3

As discussed herein, the inventors have discovered that inhibition of NSD3 can be used in the methods and compositions as disclosed herein. In some embodiments, an inhibitor of NSD3 is a protein inhibitor, and in some embodiments, the inhibitor is any agent which inhibits the function of NSD3 or the expression of Chk2 or PDE1 from its gene. In some embodiments, an inhibitor of NSD3 is a gene silencing agent.

Without wishing to be bound by theory, NSD3 is a methyltransfersase protein and is a BET-binding protein and is also known as WHSC1L1 (histone methyl-transferase) or FLJ20353. NSD3 belongs to the SET Domain Containing (NSD) protein family, which includes NSD1 and NSD2 (WHSC1/MMSET) SET domain-containing methyltransferases.

NSD3, and is encoded by nucleic acid sequence NM_023034.1 (SEQ ID NO: 1), and has an amino acid of NP_075447.1 (SEQ ID NO: 2) Inhibition of the NSD3 gene can be by gene silencing RNAi molecules according to methods commonly known by a skilled artisan. For example, a gene silencing siRNA oligonucleotide duplexes targeted specifically to human NSD3 (GenBank No: NM_023034.1; SEQ ID NO: 1) can readily be used to knockdown NSD3 expression. NSD3 mRNA can be successfully targeted using siRNAs; and other siRNA molecules may be readily prepared by those of skill in the art based on the known sequence of the target mRNA. Accordingly, in avoidance of any doubt, one of ordinary skill in the art can design nucleic acid inhibitors, such as RNAi (RNA silencing) agents to the nucleic acid sequence of NM_023034.1 which is as follows:

| (SEQ ID NO: 1) | |
|---|---|
| 1 | gggggctttg tgcgcggcgg cggcgggaga ggcggcggcg gcggccagca cggaggcgga |
| 61 | ggccgagggg gctgtgcaca ggtcgccgcg gagaggcgtg cgaattccga gccgagcgcc |
| 121 | gaggaccgtg ctacccaggc cgggctgcca gccgcaggct cctctctggc agcagcggcg |
| 181 | gcgcggcgac ccccgtccct cggcctcccc ttcccatccc acctcccgag ccttcctctt |
| 241 | cccgcagcac gcccggcccg gcccggccgt ggccctcctc agtgccggcc gccatggcag |
| 301 | aggcgtccgg cgcggggaaa atctagcccg gggatttcat gcggcctagc tcggttccgc |
| 361 | ctcctcctcg cgcggcccca gcggctgccc gcaccccagc cccactccgg gcctccgtgt |
| 421 | ctctcctgtg atcgcactga cacggccggg gggttagaat ggaacaaact gaaggcccga |
| 481 | tgagagaaag ggaaagttaa ggatgctgga gcagaacaat ggatttctct ttctctttca |
| 541 | tgcaagggat catgggaaac acaattcagc aaccacctca actcattgac tccgccaaca |
| 601 | tccgtcagga ggatgccttt gataacaaca gtgacattgc tgaagatggt ggccagacac |
| 661 | catatgaagc tactttgcag caaggctttc agtacccagc tacaacagaa gatcttcctc |
| 721 | cactcacaaa tgggtatcca tcatcaatca gtgtgtatga aactcaaacc aaataccagt |
| 781 | catataatca gtatcctaat gggtcagcca atggctttgg tgcagttaga aactttagcc |
| 841 | ccactgacta ttatcattca gaaattccaa acacaagacc acatgaaatt ctggaaaaac |
| 901 | cttcccctcc acagccacca cctcctcctt cggtaccaca aactgtgatt ccaaagaaga |
| 961 | ctggctcacc tgaaattaaa ctaaaaataa ccaaaactat ccagaatggc agggaattgt |
| 1021 | ttgagtcttc cctttgtgga gaccttttaa atgaagtaca ggcaagtgag cacacgaaat |
| 1081 | caaagcatga aagcagaaaa gaaaagagga aaaaaagcaa caagcatgac tcatcaagat |
| 1141 | ctgaagagcg caagtcacac aaaatcccca aattagaacc agaggaacaa aatagaccaa |
| 1201 | atgagagggt tgacactgta tcagaaaaac caagggaaga accagtacta aaagaggaag |
| 1261 | ccccagttca gccaatacta tcttctgttc caacaacgga agtgtccact ggtgttaagt |
| 1321 | ttcaggttgg cgatcttgtg tggtccaagg tgggaaccta tccttggtgg ccttgtatgg |
| 1381 | tttcaagtga tccccagctt gaggttcata ctaaaattaa cacaagaggt gcccgagaat |
| 1441 | atcatgtcca gttttttagc aaccagccag agagggcgtg ggttcatgaa aaacgggtac |
| 1501 | gagagtataa aggtcataaa cagtatgaag aattactggc tgaggcaacc aaacaagcca |
| 1561 | gcaatcactc tgagaaacaa aagattcgga aaccccgacc tcagagagaa cgtgctcagt |
| 1621 | gggatattgg cattgcccat gcagagaaag cattgaaaat gactcgagaa gaaagaatag |
| 1681 | aacagtatac ttttatttac attgataaac agcctgaaga ggctttatcc caagcaaaaa |
| 1741 | agagtgttgc ctccaaaacc gaagttaaaa aaacccgacg accaagatct gtgctgaata |
| 1801 | ctcagccaga acagaccaat gcaggggagg tggcctcctc actctcaagt actgaaattc |
| 1861 | ggagacatag ccagaggcgg cacacaagtg cggaagagga agagccaccg cctgttaaaa |
| 1921 | tagcctggaa aactgcggca gcaaggaaat ccttaccagc ttccattacg atgcacaaag |
| 1981 | ggagcctgga tttgcagaag tgtaacatgt ctccagttgt gaaaattgaa caagtgtttg |
| 2041 | ctcttcagaa tgctacaggg gatgggaaat ttatcgatca atttgtttat tcaacaaagg |
| 2101 | gaattggtaa caaaacagaa ataagtgtca gggggcaaga caggcttata atttctacac |
| 2161 | caaaccagag aaatgaaaag ccaacgcaga gtgtatcatc tcctgaagca acatctggtt |
| 2221 | ctacaggctc agtagaaaag aagcaacaga gaagatcaat tagaactcgt tctgaatcag |
| 2281 | agaaatccac tgaggttgtg ccaaagaaga agatcaaaaa ggagcaggtt gaaacagttc |
| 2341 | ctcaggctac agtgaagact ggattacaga aaggtgccag cgagatttca gattcctgta |
| 2401 | aacctctaaa gaaaaggagt cgcgcctcaa ctgatgtaga aatgactagt tcagcataca |
| 2461 | gagacacatc tgactccgat tctagaggac tgagtgacct gcaggtaggc tttggaaagc |
| 2521 | aagtagatag cccttcagct actgcagatg cagacgtttc tgatgtgcag tccatggatt |
| 2581 | caagtttgtc gagaagaggc actggaatga gtaagaagga cactgtatgt cagatttgtg |
| 2641 | aaagctctgg tgactctctg attccttgtg agggagagtg ctgcaaacac tttcacctgg |
| 2701 | agtgcctggg attggcatca cttcctgata gcaagttcat ctgcatggaa tgtaaaactg |
| 2761 | ggcagcaccc atgtttttcg tgtaaagtgt ctggtaaaga tgtgaagcgt tgttctgttg |
| 2821 | gtgcttgtgg gaaattttat catgaagcct gtgtccgcaa attccccact gccatctttg |
| 2881 | aatcaaaagg attccgctgt cctcagcact gctgctctgc ctgctctatg gagaaagata |
| 2941 | tccacaaagc aagtaaaggc cgcatgatga gatgtttaag atgtccagtt gcctatcact |
| 3001 | ctggagatgc ttgcattgcg gccggaagca tgttagtatc ctcctacatt ctcatctgta |
| 3061 | gtaatcattc caaacggagc agtaattctt ctgctgtaaa tgtaggcttt tgtttcgttt |
| 3121 | gtgccagagg gctgatagtt caggaccatt cagaccccat gttcagttca tatgcctata |
| 3181 | agtcccacta cctactgaat gaatcaaatc gtgctgagtt gatgaaatta cctatgattc |
| 3241 | cttcttcgtc agcttccaaa aagaaatgtg agaaaggtgg aagattgctc tgctgtgaat |
| 3301 | cgtgcccagc ttccttccac ccggaatgcc taagcataga aatgccagaa ggctgctgga |
| 3361 | attgtaatga ctgtaaagct ggcaagaaac tacattacaa gcagattgtt tgggtcaaat |
| 3421 | tgggaaatta cagatggtgg ccagcagaga tctgcaaccc caggtctgtg ccactgaaca |
| 3481 | tccagggcct taaacatgac ttgggggact tccctgtatt cttctttggt tctcatgact |
| 3541 | actactgggt acaccagggc agagtgttcc cttatgttga aggagacaaa agctttgctg |
| 3601 | aagggcagac tagtattaac aagaccttca aaaaggcact ggaagaagct gcaaaacgtt |
| 3661 | tccaggaatt gaaagcacaa agagaaagta aagaagccct agagattgaa aaaaactcaa |
| 3721 | gaaaaccccc tccctacaaa cacatcaaag ctaacaaagt aataggaaag gtgcagatcc |
| 3781 | aggttgctga cctgtcagag attccccgct gtaactgcaa gccagctgat gaaaaccctt |
| 3841 | gtggcttgga atcggagtgc ctgaacagaa tgttgcagta tgaatgccac ccgcaggtgt |
| 3901 | gcccagctgg agatcgttgt cagaaccagt gctttacaaa gagactatac cctgatgcag |
| 3961 | agatcatcaa aacggagcgg agaggctggg gcctcaggac caaaaggagc attaagaagg |
| 4021 | gtgaatttgt aaatgaatac gtcggtgaat taattgatga agaagaatgc agattgcgaa |
| 4081 | tcaagcgagc ccacgagaac agtgtaacta atttttatat gttaactgtt accaaggacc |
| 4141 | gtataattga tgccggccca aaaggaaatt attctcgctt catgaaccac agttgtaatc |
| 4201 | ccaactgtga aacacaaaag tggacagtga atggagatgt tcgagtggga ctatttgctc |
| 4261 | tctgtgatat tcctgcaggg atggagttaa catttaatta taacctagat tgtctgggca |
| 4321 | acggcagaac ggagtgccac tgtggagcag ataactgcag tggttttcta ggagtgcggc |
| 4381 | caaagtcggc atgtgcgtca acaaatgaag agaaggcaaa aaatgctaag ttaaaacaga |
| 4441 | agagacgaaa gatcaaaaca gaaccaaagc agatgcatga agattactgt tttcaatgtg |
| 4501 | gagatggtgg agagctggtc atgtgtgaca aaaaagactg tcccaaagca taccacctcc |
| 4561 | tatgccttaa cctgactcag ccaccatatg gaaagtggga gtgtccgtgg catcagtgcg |
| 4621 | atgagtgcag cagtgcagct gtttccttct gtgaattctg tccacattca ttttgtaaag |
| 4681 | atcatgaaaa gggggccctg gttccctctg cactggaagg ccgcctctgc tgctcggaac |
| 4741 | atgaccccat ggctcctgtg tcaccagaat actggagcaa gataaaatgt aaatgggaat |
| 4801 | cacaagatca tggagaagaa gtaaaagaat aaatgtgtgg tgtcccctcc tttctattta |
| 4861 | agtgaaaaaa gcaaatagat catgcattta aaaagaagag actgctacag tgcatacagc |
| 4921 | ctttgccatc ggaactgcct tattaaagca aaaatgggaa accagttcat gcaggcagaa |
| 4981 | gcagttggtg gtgtctggtt tttgtttgat ttggttggtt tgggattctt ttgtggaggg |
| 5041 | ttaaattccc ttggtctttt cttgcctttt attgtgcttc agtgccattg cagcttgaaa |
| 5101 | aagaaatgtt tttgctgtta aaataagaac aaagagaaaa gtaagttttg ttaatgagat |
| 5161 | aaatttaaag tctaagatgt gttccttggt tgtataaagc aaaagtagcc atcattcctt |
| 5221 | tatttatttt catttttagg aatttcaaga agtgtagttc aatagtctaa tcaagtgtgt |
| 5281 | gtgtgtttta agtaggaatc tgagaaagcc ctctaggaaa gggtatgata agctttatat |
| 5341 | acctctttac tgagcagtag gtaggctcac ttctctttcc cttcaaaatg cttttcatag |
| 5401 | tagagaa gggctctatg gaagtattaa a |

In some embodiments, a NSD3 inhibitor is a siRNA which targets the NSD3 portion which binds to the ET domain of BRD4. Accordingly, one of ordinary skill in the art can design nucleic acid inhibitors, such as RNAi (RNA silencing) agents to the nucleic acid sequence of SEQ ID NO: 3 which is the portion of NSD3 mRNA which encodes the region of NSD3 which binds to the ET domain of BRD4. The amino acid sequence of the portion of NSD3 protein which binds to the ET domain of BRD4 comprises all, or a portion of SEQ ID NO: 4 and is encoded by the nucleic acid sequence of SEQ ID NO: 3 which is as follows:

(SEQ ID NO: 3)
ATGGATTTCTCTTTCTCTTTCATGCAAGGGATCATGGGAAACACAATT
CAGCAACCACCTCAACTCATTGACTCCGCCAACATCCGTCAGGAGGAT
GCCTTTGATAACAACAGTGACATTGCTGAAGATGGTGGCCAGACACCA
TATGAAGCTACTTTGCAGCAAGGCTTTCAGTACCCAGCTACAACAGAA
GATCTTCCTCCACTCACAAATGGGTATCCATCATCAATCAGTGTGTAT
GAAACTCAAACCAAATACCAGTCATATAATCAGTATCCTAATGGGTCA
GCCAATGGCTTTGGTGCAGTTAGAAACTTTAGCCCCACTGACTATTAT
CATTCAGAAATTCCAAACACAAGACCACATGAAATTCTGGAAAAACCT
TCCCCTCCACAGCCACCACCTCCTCCTTCGGTACCACAAACTGTGATT
CCAAAGAAGACTGGCTCACCTGAAATTAAACTAAAAATAACCAAAACT
ATCCAGAATGGCAGGGAATTGTTTGAGTCTTCCCTTTGTGGAGACCTT
TTAAATGAAGTACAGGCAAGTGAGCACACGAAATCAAAGCATGAAAGC
AGAAAAGAAAAGAGGAAAAAAAGCAACAAGCATGACTCATCAAGATCT
GAAGAGCGCAAGTCACACAAAATCCCCAAATTAGAACCAGAGGAACAA
AATAGACCAAATGAGAGGGTTGACACTGTATCAGAAAAACCAAGGGAA
GAACCAGTACTAAAAGAGGAAGCCCCAGTTCAGCCAATACTATCTTCT
GTTCCAACAACGGAAGTGTCCACTGGTGTTAAGTTTCAGGTTGGCGAT
CTTGTGTGGTCCAAGGTGGGAACCTATCCTTGGTGGCCTTGTATGGTT
TCAAGTGATCCCCAGCTTGAGGTTCATACTAAAATTAACACAAGAGGT
GCCCGAGAATATCATGTCCAGTTTTTTAGCAACCAGCCAGAGAGGGCG
TGGGTTCATGAAAAACGGGTACGAGAGTATAAAGGTCATAAACAGTAT
GAAGAATTACTGGCTGAGGCAACCAAACAAGCCAGCAATCACTCTGAG
AAACAAAAGATTCGGAAACCCCGACCTCAGAGAGAACGTGCTCAGTGG
GATATTGGCATTGCCCATGCAGAGAAAGCATTGAAAATGACTCGAGAA
GAAAGAATAGAACAGTATACTTTTATTTACATTGATAAACAGCCTGAA
GAGGCTTTATCCCAAGCAAAAAAGAGTGTTGCCTCCAAAACCGAAGTT
AAAAAAACCCGACGACCAAGATCTGTGCTGAATACTCAGCCAGAACAG
ACCAATGCAGGGGAGGTGGCCTCCTCACTCTCAAGTACTGAAATTCGG
AGACATAGCCAGAGGCGGCACACAAGTGCGGAAGAGGAAGAGCCACCG
CCTGTTAAAATAGCCTGGAAAACTGCGGCAGCAAGGAAATCCTTACCA
GCTTCCATTACGATGCACAAAGGGAGCCTGGATTTGCAGAAGTGTAAC
ATGTCTCCAGTTGTGAAAATTGAACAAGTGTTTGCTCTTCAGAATGCT
ACAGGGGATGGGAAATTTATCGATCAATTTGTTTATTCAACAAAGGGA
ATTGGTAACAAAACAGAAATAAGTGTCAGGGGGCAAGACAGGCTTATA
ATTTCTACACCAAACCAGAGAAATGAAAAGCCAACGCAGAGTGTATCA
TCTCCTGAAGCAACATCTGGTTCTACA

The amino acid sequence of SEQ ID NO: 4 (a portion of NSD3 that binds ET domain on BRD4) is as follows:

MDFSFSFMQGIMGNTIQQPPQLIDSANIRQEDAFDNNSDIAEDGGQTP
YEATLQQGFQYPATTEDLPPLTNGYPSSISVYETQTKYQSYNQYPNGS
ANGFGAVRNFSPTDYYHSEIPNTRPHEILEKPSPPQPPPPPSVPQTVI
PKKTGSPEIKLKITKTIQNGRELFESSLCGDLLNEVQASEHTKSKHES
RKEKRKKSNKHDSSRSEERKSHKIPKLEPEEQNRPNERVDTVSEKPRE
EPVLKEEAPVQPILSSVPTTEVSTGVKFQVGDLVWSKVGTYPWWPCMV
SSDPQLEVHTKINTRGAREYHVQFFSNQPERAWVHEKRVREYKGHKQY
EELLAEATKQASNHSEKQKIRKPRPQRERAQWDIGIAHAEKALKMTRE
ERIEQYTFIYIDKQPEEALSQAKKSVASKTEVKKTRRPRSVLNTQPEQ
TNAGEVASSLSSTEIRRHSQRRHTSAEEEEPPPVKIAWKTAAARKSLP
ASITMHKGSLDLQKCNMSPVVKIEQVFALQNATGDGKFIDQFVYSTKG
IGNKTEISVRGQDRLIISTPNQRNEKPTQSVSSPEATSGST.

In some embodiments, a NSD3 inhibitor is a commercially available siRNA, such as available from Santa Cruz (cat # sc-61235). In some embodiments, siRNA or RNAi targeting NSD3 can be delivered in a vector, for example a lentiviral vector, such as commercially available NSD3 shRNA lentivial vectors from Santa Cruz (cat # sc-61235-V). In some embodiments, NSD3 inhibitor are as follows: siNSD3-6 ON-TARGETplus Human WHSC1L1 (54904) (Dharmacon Cat # J-012875-06), siNSD3-7, ON-TARGETplus Human WHSC1L1 (54904) siRNA (Dharmacon Cat # J-012875-07), siNSD3 3′-1 CUGUAAACCUCUAAAGAAAUU (SEQ ID NO: 15), si NSD3 3′-2 GAAAGGUGCCAGCGAGAUUUU (SEQ ID NO: 16), siNSD3-06 GAACGUGCUCAGUGGGAUA (Dharmacon Cat # J-J-012875-06-0020) (SEQ ID NO: 17), siNSD3-07 GCUUGAGGUUCAUACUAAA (Dharmacon Cat # J-012875-07-0020) (SEQ ID NO: 18).

In some embodiments, a NSD3 inhibitor is an antibody or antibody fragment. Antibodies and antibody fragments are well known in the art, and are commercially available. In some embodiments, a NSD3 antibody is available from Santa Cruz (2E9, cat # sc-130009).

Also siRNA/RNAi, antisense molecules and ribozymes directed against nucleic acid molecules encoding NSD3 are envisaged as NSD3i for the use and the method of the present invention. The above-mentioned antagonist/inhibitor of NSD3 may also be a co-suppressive nucleic acid.

An siRNA approach is, for example, disclosed in Elbashir ((2001), Nature 41 1, 494-498)). It is also envisaged in accordance with this invention that for example short hairpin RNAs (shRNAs) are employed in accordance with this invention as pharmaceutical composition. The shRNA approach for gene silencing is well known in the art and may comprise the use of st (small temporal) RNAs; see, inter alia, Paddison (2002) Genes Dev. 16, 948-958.

As mentioned above, approaches for gene silencing are known in the art and comprise “RNA”-approaches like RNAi (iRNA) or siRNA. Successful use of such approaches has been shown in Paddison (2002), Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002); Miyagishi (2002) or Brummelkamp (2002).

In some embodiments, a RNAi or siRNA is targeted to a tumor or cancer cell. In some embodiments, the NSD3i RNAi agent, e.g., an anti-NSD3 RNAi complementary to, or complementary in part, to the NSD3 of SEQ ID NO: 1 or SEQ ID NO: 3, can further comprise a binding moiety and a targeting moiety, and in some embodiments the binding moiety binds the NSD3i RNAi agent to the targeting moiety. In some embodiments, a targeting moiety is a cell surface receptor ligand or antigen-binding fragment thereof, for example a cell surface receptor or ligand which is expressed on cells expressing the NSD3/NUT or BRD4/NUT or BRD3/NUT fusion gene.

In some embodiments, a targeting moiety useful in the methods as disclosed herein is an antibody, for example an antibody including not just complete or full length antibodies, but also antibody derivatives, such as a single chain antibody, a Fab portion of an antibody or a (Fab′)₂ segment. In some embodiments, a binding moiety useful in the methods as disclosed herein is a protein or a nucleic acid binding domain of a protein, and in some embodiments the binding moiety is fused to the carboxyl terminus of the targeting moiety, and in some embodiments, the binding moiety is the protein protamine or nucleic acid binding fragment of protamine.

In some embodiments, an NSD3i agent, e.g., a peptide inhibitor of NSD3 or a NSD3i RNAi agent, e.g., an anti-NSD3 RNAi complementary to, or complementary in part, to the NSD3 of SEQ ID NO: 1 or SEQ ID NO: 3 is encoded by a nucleic acid in a vector, for example, a plasmid, cosmid, phagemid, or virus or variants thereof, and in some embodiments the NSD3i agent, e.g., a peptide inhibitor of NSD3 or a NSD3i RNAi agent is operatively linked to a promoter. In some embodiments, the vector further comprises one or more in vivo expression elements for expression in human cells, such as a promoter or enhancer and combinations thereof.

In some embodiments, administration of a NSD3i agent, e.g., a peptide inhibitor of NSD3 or a NSD3i RNAi agent, e.g., an anti-NSD3 RNAi complementary to, or complementary in part, to the NSD3 of SEQ ID NO: 1 or SEQ ID NO: 3 can be intravenous, intradermal, intramuscular, intraarterial, intralesional, percutaneous, subcutaneous, or by aerosol administration, or combinations thereof. In some embodiments, administration is prophylactic administration, and in alternative embodiments, administration is therapeutic administration. Anitmirs have been effective in vivo to block miRNA mediated gene suppression when administered a variety of ways, in particular, intravenous, subcutaneous, interperatenial (i.p) and other administration routes. In some embodiments, where the anti-miR-425 agent is a locked nucleic acid (LNA), the LNA is administered to a subject intravenously, for example at a dose of about 10 mg/kg, or at least about 2 mg/kg, or at about at least 5 mg/kg, or at least about 10 mg/kg. Intravenous administration of LNA has been demonstrated to be effective to inhibit miRNA mediated gene suppression in vivo (Obad et al, Nature Genetics, 2011; 43; 371-378, which is incorporated herein in its entirety by reference).

In some embodiments, the methods and compositions as disclosed herein can be administered to a subject, where the subject is, for example, a mammal such as a human.

In some embodiments, a NSD3 inhibitor agent as disclosed herein can be, for example a small molecule, nucleic acid, nucleic acid analogue, aptamer, ribozyme, peptide, protein, antibody, or variants and fragments thereof. In some embodiments, a nucleic acid agent can be DNA, RNA, nucleic acid analogue, peptide nucleic acid (PNA), pseudo-complementary PNA (pcPNA), locked nucleic acid (LNA) or analogue thereof, and in embodiments where the nucleic acid agent is RNA, the RNA can be a small inhibitory RNA (RNAi), siRNA, microRNA, shRNA, miRNA and analogues and homologues and variants thereof effective in gene silencing. In some embodiments, a NSD3i is a LNA oligonucleotide which is complementary to, or complementary in part, to the NSD3 of SEQ ID NO: 1 or SEQ ID NO: 3.

In some embodiments, a NSD3i agent is a Tiny LNA oligonucleotide which is complementary to at least part of SEQ ID NO: 1 or SEQ ID NO: 3.

In some embodiments, a NSD3i is an antagomir, fully 2′-O-methoxyethyl (2′-MOE), 2′-F/MOE mixmer, LNA/DNA mixmer, a tiny LNA or a combination thereof, which are complementary to, or complementary in part, to of SEQ ID NO: 1 or SEQ ID NO: 3. As used herein, the term “tiny LNA” refers to a short, e.g., 6, 7, 8, 9, 10, 11 or 12-mer oligonucleotide that is comprised entirely of locked nucleic acid monomers. Tiny LNAs are described in Obad et al., (Nature Genetics, 2010, 43(4): 371-380, content of which is incorporated herein by reference. In some embodiments, the tiny LNA comprises phosphorothioate inter-sugar linkages at all positions. In some embodiments, the tiny LNA is 8 nucleotides in length and comprises phosphorothioate inter-sugar linkages at all positions.

In some embodiments, a NSD3i agent comprises a modification selected from the group consisting of nucleobase modifications, sugar modifications, inter-sugar linkage modifications, backbone modifications, and any combinations thereof. In some embodiments, a NSD3i agent comprises a ligand. In some embodiments, a NSD3i agent is from about 11 to about 30 nucleotides in length. In some embodiments, a NSD3i agent is single-stranded. In some embodiments, an NSD3i agent is formulated in a lipid delivery vehicle, e.g., liposomes, lipid particles, other compositions used for oligonucleotide delivery. In some embodiments, a NSD3i agent is encoded by an expression vector.

As used herein, the term “oligonucleotide” refers to a polymer or an oligomer of nucleotide or nucleoside monomers consisting of naturally occurring bases, sugars and intersugar linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

The oligonucleotide can be single-stranded or double-stranded. A single-stranded oligonucleotide can have double-stranded regions and a double-stranded oligonucleotide can have single-stranded regions. The oligonucleotide can have a hairpin structure or have a dumbbell structure. The oligonucleotide can be, e.g., wherein the 5′end of the oligonucleotide is linked to the 3′ end of the oligonucleotide.

The oligonucleotides described herein can comprise any oligonucleotide modification described herein and below. In some embodiments, the oligonucleotide comprises at least one modification. In some embodiments, the modification is selected from the group consisting of a sugar modification, a non-phosphodiester inter-sugar (or inter-nucleoside) linkage, nucleobase modification, and ligand conjugation.

In some embodiments, the oligonucleotide comprises at least two different modifications selected from the group consisting of a sugar modification, a non-phosphodiester inter-sugar linkage, nucleobase modification, and ligand conjugation. In some embodiments, the at least two different modifications are present in the same subunit of the oligonucleotide, e.g. present in the same nucleotide.

As used herein, an oligonucleotide can be of any length. In some embodiments, oligonucleotides can range from about 6 to 100 nucleotides in length. In various related embodiments, the oligonucleotide can range in length from about 10 to about 50 nucleotides, from about 10 to about 35 nucleotides, from about 15 to about 30 nucleotides, from about 20 to about 30 nucleotides in length. In some embodiments, oligonucleotide is from about 8 to about 39 nucleotides in length. In some embodiments, the oligonucleotide is 10 to 25 nucleotides in length (e.g., 10, 11, 12, 13, 14, 15, 16, 18, 19, 20, 21, 22, 23, or 24 nucleotides in length). In some embodiments the oligonucleotide is 25-30 nucleotides. In some embodiments, the single-stranded oligonucleotide is 15 to 29 nucleotides in length. In some other embodiments, the oligonucleotide is from about 18 to about 25 nucleotides in length. In some embodiments, the oligonucleotide is about 23 nucleotides in length.

The oligonucleotide can be completely DNA, completely RNA, or comprise both RNA and DNA nucleotides. It is to be understood that when the oligonucleotide is completely DNA, RNA or a mix of both, the oligonucleotide can comprise one or more oligonucleotide modifications described herein.

An oligonucleotide can be a chimeric oligonucleotide. As used herein, a “chimeric” oligonucleotide” or “chimera” refers to an oligonucleotide which contains two or more chemically distinct regions, each made up of at least one monomer unit, i.e., a modified or unmodified nucleotide in the case of an oligonucleotide. Chimeric oligonucleotides can be described as having a particular motif. In some embodiments, the motifs include, but are not limited to, an alternating motif, a gapped motif, a hemi-mer motif, a uniformly fully modified motif and a positionally modified motif. As used herein, the phrase “chemically distinct region” refers to an oligonucleotide region which is different from other regions by having a modification that is not present elsewhere in the oligonucleotide or by not having a modification that is present elsewhere in the oligonucleotide. An oligonucleotide can comprise two or more chemically distinct regions. As used herein, a region that comprises no modifications is also considered chemically distinct.

A chemically distinct region can be repeated within an oligonucleotide. Thus, a pattern of chemically distinct regions in an oligonucleotide can be realized such that a first chemically distinct region is followed by one or more second chemically distinct regions. This sequence of chemically distinct regions can be repeated one or more times. Preferably, the sequence is repeated more than one time. Both strands of a double-stranded oligonucleotides can comprise these sequences. Each chemically distinct region can actually comprise as little as a single nucleotide. In some embodiments, each chemically distinct region comprises 1, 2, 3, 4, 5, 6, 7, 8, 9, 10, 11, 12, 13, 14, 15, 16, 17 or 18 nucleotides.

In some embodiments, alternating nucleotides comprise the same modification, e.g. all the odd number nucleotides in a strand have the same modification and/or all the even number nucleotides in a strand have the similar modification to the first strand. In some embodiments, all the odd number nucleotides in an oligonucleotide have the same modification and all the even numbered nucleotides have a modification that is not present in the odd number nucleotides and vice versa.

When the oligonucleotide is double-stranded and both strands of the double-stranded oligonucleotide comprise the alternating modification patterns, nucleotides of one strand can be complementary in position to nucleotides of the second strand which are similarly modified. In an alternative embodiment, there is a phase shift between the patterns of modifications of the first strand, respectively, relative to the pattern of similar modifications of the second strand. Preferably, the shift is such that the similarly modified nucleotides of the first strand and second strand are not in complementary position to each other. In some embodiments, the first strand has an alternating modification pattern wherein alternating nucleotides comprise a 2′-modification, e.g., 2′-O-Methyl modification. In some embodiments, the first strand comprises an alternating 2′-O-Methyl modification and the second strand comprises an alternating 2′-fluoro modification. In other embodiments, both strands of a double-stranded oligonucleotide comprise alternating 2′-O-methyl modifications. When both strands of a double-stranded oligonucleotide comprise alternating 2′-O-methyl modifications, such 2′-modified nucleotides can be in complementary position in the duplex region. Alternatively, such 2′-modified nucleotides may not be in complementary positions in the duplex region.

In some embodiments, the oligonucleotide comprises two chemically distinct regions, wherein each region is 1-10 nucleotides in length.

In other embodiments, the oligonucleotide comprises three chemically distinct regions. The middle region is about 5-15, (e.g., 5, 6, 7, 8, 9, 10, 11, 12, 13, 14 or 15) nucleotide in length and each flanking or wing region is independently 1-10 (e.g., 1, 2, 3, 4, 5, 6, 7, 8, 9 or 10) nucleotides in length. All three regions can have different modifications or the wing regions can be similarly modified to each other. In some embodiments, the wing regions are of equal length, e.g. 1, 2, 3, 4, 5, 6, 7, 8, 9 or 10 nucleotides long.

As used herein the term “alternating motif” refers to an oligonucleotide comprising at least two different chemically distinct regions that alternate for essentially the entire sequence of the oligonucleotide. In an alternating motif length of each region is independent of the length of other regions.

As used herein, the term “uniformly fully modified motif” refers to an oligonucleotide wherein all nucleotides in the oligonucleotide have at least one modification that is the same.

As used herein, the term “hemi-mer motif” refers to an oligonucleotide having two chemically distinct regions, wherein one region is at the 5′ end of the oligonucleotide and the other region is at the 3 end of the oligonucleotide. In some embodiments, length of each chemically distinct region is independently 1 nucleotide to 1 nucleotide less than the length of the oligonucleotide.

As used herein the term “gapped motif” refers to an oligonucleotide having three chemically distinct regions. In some embodiments, the gapped motif is a symmetric gapped motif, wherein the two outer chemically distinct regions (wing regions) are identically modified. In another embodiment, the gapped motif is an asymmetric gaped motif in that the three regions are chemically distinct from each other

As used herein the term “positionally modified motif” refers to an oligonucleotide having three or more chemically distinct regions. Positionally modified oligonucleotides are distinguished from gapped motifs, hemi-mer motifs, blockmer motifs and alternating motifs because the pattern of regional substitution defined by any positional motif does not fit into the definition provided herein for one of these other motifs. The term positionally modified oligomeric compound includes many different specific substitution patterns.

In some embodiments, oligonucleotide comprises two or more chemically distinct regions and has a structure as described in International Application No. PCT/US09/038433, filed Mar. 26, 2009, content of which is incorporated herein by reference in its entirety. In some embodiments, the single-stranded oligonucleotide has a ZXY structure, such as is described in International Application No. PCT/US2004/07070 filed on Mar. 8, 2004, content of which is incorporated herein by reference in its entirety.

In some embodiments, the anti-miR oligonucleotide comprises 2′-MOE modifications at all positions and phosphorothioate inter-sugar linkages at all positions.

In some embodiments, the anti-miR comprises a mix of 2′-F and 2′-MOE modified nucleotides.

In some embodiments, the anti-miR comprises at least 1 (e.g., 1, 2, 3, 4, or 5) 2′-F modified nucleotides at the 5′ end (i.e., the first 1, 2, 3, 4, or 5 nucleotides at the 5′ end are 2′-F modified nucleotides).

In some embodiments, the anti-miR comprises at least 1 (e.g., 1, 2, 3, 4, or 5) 2′-F modified nucleotides at the 3′ end (i.e., the first 1, 2, 3, 4, or 5 nucleotides at the 3′ end are 2′-F modified nucleotides).

In some embodiments, the anti-miR comprises, independently, at least 1 (e.g., 1, 2, 3, 4, or 5) 2′-F modified nucleotides at the 5′ end and at the 3′ end and 2′-MOE modified nucleotides at all other positions.

In some embodiments, the anti-miR comprises two 2′-F modified nucleotides at the 5′ end and at the 3′ end and 2′-MOE modified nucleotides at all other positions, e.g., a 2′-F/2′-MOE mixmer.

In some embodiments, the anti-miR comprises two 2′-F modified nucleotides at the 5′ end and at the 3′ end, 2′-MOE modified nucleotides at all other positions, and phosphorothioate inter-sugar linkages at all positions.

In some embodiments, the anti-miR comprises a mix of LNA and DNA monomers, e.g., a LNA/DNA mixmer. The LNA and DNA monomers can be arranged in any pattern. In some embodiments, the LNA and DNA monomers are arranged in an alternative pattern, e.g., a LNA monomer followed by a DNA monomer. This alternating pattern can be repeated for the full length of the anti-miR.

The oligonucleotide can hybridize to a complementary RNA, e.g., mRNA, pre-mRNA, microRNA, or pre-microRNA and reduce the activity, expression, or amount of the complementary RNA, e.g., target RNA. This can be by reducing access of the translation machinery to the target RNA transcript, thereby preventing protein synthesis. The oligonucleotide can induce cleavage of the complementary RNA by an enzyme, such RISC mediated cleavage or RNase H and thus reducing the amount of the target RNA. The oligonucleotide itself can cleave the complementary RNA, e.g., a ribozyme, RISC mediated cleavage or RNase H and thus reducing the amount of the target RNA. The oligonucleotide, by hybridizing to the target RNA, can inhibit binding of the target RNA to another complementary strand.

By “target sequence” is meant any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant.

In some embodiments, the target sequence of a RNAi targets a portion of the nucleic acid encoding, expressing or comprising the nucleotide sequence of NSD3 of SEQ ID NO: 1 or a fragment thereof of SEQ ID NO: 3.

By “specifically hybridizable” and “complementary” is meant that a first nucleic acid strand can form hydrogen bond(s) with a second nucleic acid strand by either traditional Watson-Crick or other non-traditional types. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be non-complementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically differ by at least 1, 2, 3, 4, or 5 nucleotides.

MicroRNAs:

MicroRNAs (miRNAs or mirs) are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

MicroRNAs have also been implicated in modulation of pathogens in hosts. For example, see Jopling, C. L., et al., Science (2005) vol. 309, pp 1577-1581. Without wishing to be bound by theory, administration of a microRNA, microRNA mimic, and/or anti microRNA oligonucleotide, leads to modulation of pathogen viability, growth, development, and/or replication. In some embodiments, the oligonucleotide is a microRNA, microRNA mimic, and/or anti microRNA, wherein microRNA is a host microRNA.

The number of miRNA sequences identified to date is large and growing, illustrative examples of which can be found, for example, in: “miRBase: microRNA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S. NAR, 2004, 32, Database Issue, D109-D111; and also on the worldwide web at microrna.dot.sanger.dot.ac.dot.uk/sequences/.

miRNA Mimics:

miRNA mimics represent a class of molecules that can be used to imitate the gene modulating activity of one or more miRNAs. Thus, the term “microRNA mimic” refers to synthetic non-coding RNAs (i.e. the miRNA is not obtained by purification from a source of the endogenous miRNA) that are capable of entering the RNAi pathway and regulating gene expression. miRNA mimics can be designed as mature molecules (e.g. single stranded) or mimic precursors (e.g., pri- or pre-miRNAs).

In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded oligonucleotides.

In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

Supermirs:

A supermir refers to an oligonucleotide, e.g., single stranded, double-stranded or partially double-stranded, which has a nucleotide sequence that is substantially identical to an miRNA and that is antisense with respect to its target. This term includes oligonucleotides which comprise at least one non-naturally-occurring portion which functions similarly. In a preferred embodiment, the supermir does not include a sense strand, and in another preferred embodiment, the supermir does not self-hybridize to a significant extent. A supermir featured in the invention can have secondary structure, but it is substantially single-stranded under physiological conditions. A supermir that is substantially single-stranded is single-stranded to the extent that less than about 50% (e.g., less than about 40%, 30%, 20%, 10%, or 5%) of the supermir is duplexed with itself. The supermir can include a hairpin segment, e.g., sequence, preferably at the 3′ end can self hybridize and form a duplex region, e.g., a duplex region of at least 1, 2, 3, or 4 and preferably less than 8, 7, 6, or 5 nucleotides, e.g., 5 nucleotides. The duplexed region can be connected by a linker, e.g., a nucleotide linker, e.g., 3, 4, 5, or 6 dTs, e.g., modified dTs. In another embodiment the supermir is duplexed with a shorter oligo, e.g., of 5, 6, 7, 8, 9, or 10 nucleotides in length, e.g., at one or both of the 3′ and 5′ end or at one end and in the non-terminal or middle of the supermir.

Oligonucleotide Modifications:

Unmodified oligonucleotides can be less than optimal in some applications, e.g., unmodified oligonucleotides can be prone to degradation by e.g., cellular nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

Modifications of Phosphate Group:

The phosphate group in the intersugar linkage can be modified by replacing one of the oxygens with a different substituent. One result of this modification to RNA phosphate intersugar linkages can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the intersugar linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

Replacement of the Phosphate Group:

The phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H)—C(═O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Replacement of Ribophosphate Backbone:

Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. In some embodiments, the oligonucleotide is a peptide nucleic acid, e.g., the ribophosphate backbone of the oligonucleotide is completely replaced by peptide nucleic acid (PNA).

Sugar Modifications:

An oligonucleotide can include modification of all or some of the sugar groups of the nucleic acid. For example, the 2′ position (H, DNA; or OH, RNA) can be modified with a number of different “oxy” or “deoxy” substituents. While not being bound by theory, enhanced stability is expected since the 2′-hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the oxygen at the 2′ position is connected by (CH₂)ₙ, wherein n=1-4, to the 4′ carbon of the same ribose sugar, preferably n is 1 (LNA) or 2 (ENA); O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

Examples of “deoxy” modifications include halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

The sugar group can also contain one or more carbons that possess the opposite stereochemical configuration than that of the corresponding carbon in the ribose sugar. Thus, an oligonucleotide can include nucleotides containing e.g., arabinose, as the sugar. Similarly, a modification at the 2′ position can be present in the arabinose configuration The term “arabinose configuration” refers to the placement of a substituent on the C2′ of ribose in the same configuration as the 2′-OH is in the arabinose.

A nucleotide can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Oligonucleotides can also include abasic sugars, i.e., a monomers which lack a nucleobase at C-1′ or has other chemical groups in place of a nucleobase at C1′. See for example U.S. Pat. No. 5,998,203, contents of which are herein incorporated in their entirety. These abasic sugars can also be further containing modifications at one or more of the constituent sugar atoms. Oligonucleotides can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in the a configuration.

Oligonucleotide modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

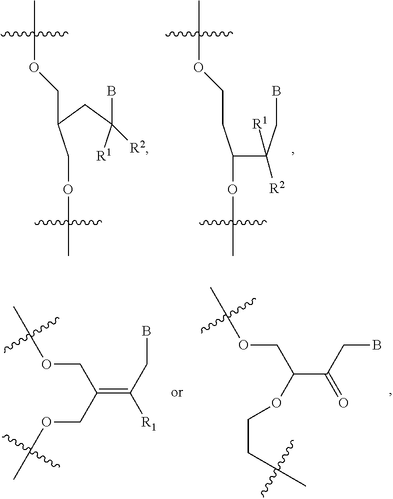

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

Preferred sugar modifications are 2′-O-Me (2′-O-methyl), 2′-O-MOE (2′-O-methoxyethyl), 2′-F, 2′-O-[2-(methylamino)-2-oxoethyl](2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), and 2′-O-dimethylaminoethyloxyethyl (2′-O-DMAEOE).

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(R′), phosphorous containing linkage, optionally substituted aryl, optionally substituted heteroaryl, optionally substituted heterocyclic or optionally substituted cycloalkyl, where R′ is hydrogen, halogen, alkyl, alkenyl, alkynyl, alkoxy, aryl, heteroaryl, cyclyl, or heterocyclyl, each of which can be optionally substituted. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the oligonucleotide.

Nucleobase Modifications:

Adenine, cytosine, guanine, thymine and uracil are the most common bases (or nucleobases) found in nucleic acids. These bases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the above modifications. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligonucleotide can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any modified or nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

Terminal Modifications:

In vivo applications of oligonucleotides can be limited due to presence of nucleases in the serum and/or blood. Thus in certain instances it is preferable to modify the 3′,5′ or both ends of an oligonucleotide to make the oligonucleotide resistant against exonucleases. In some embodiments, the oligonucleotide comprises a cap structure at 3′ (3′-cap),5′ (5′-cap) or both ends. In some embodiments, oligonucleotide comprises a 3′-cap. In another embodiment, oligonucleotide comprises a 5′-cap. In yet another embodiment, oligonucleotide comprises both a 3′ cap and a 5′ cap. It is to be understood that when an oligonucleotide comprises both a 3′ cap and a 5′ cap, such caps can be same or they can be different. As used herein, “cap structure” refers to chemical modifications, which have been incorporated at either terminus of oligonucleotide. See for example U.S. Pat. No. 5,998,203 and International Patent Publication WO03/70918, contents of which are herein incorporated in their entireties.

Exemplary 5′-caps include, but are not limited to, ligands, 5′-5′-inverted nucleotide, 5′-5′-inverted abasic nucleotide residue, 2′-5′ linkage, 5′-amino, 5′-amino-alkyl phosphate, 5′-hexylphosphate, 5′-aminohexyl phosphate, bridging and/or non-bridging 5′-phosphoramidate, bridging and/or non-bridging 5′-phosphorothioate and/or 5′-phosphorodithioate, bridging or non bridging 5′-methylphosphonate, non-phosphodiester intersugar linkage between the end two nucleotides, 4′,5′-methylene nucleotide, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, 5′-mercapto nucleotide and 5′-1,4-butanediol phosphate.

Exemplary 3′-caps include, but are not limited to, ligands, 3′-3′-inverted nucleotide, 3′-3′-inverted abasic nucleotide residue, 3′-2′-inverted nucleotide moiety, 3′-2′-inverted abasic moiety, 2′-5′-linkage, 3′-amino, 3′-amino-alkyl phosphate, 3′-hexylphosphate, 3′-aminohexyl phosphate, bridging and/or non-bridging 3′-phosphoramidate, bridging and/or non-bridging 3′-phosphorothioate and/or 3′-phosphorodithioate, bridging or non bridging 3′-methylphosphonate, non-phosphodiester intersugar linkage between the end two nucleotides, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, and 3′-1,4-butanediol phosphate. For more details see Beaucage and Iyer, 1993, Tetrahedron 49, 1925, incorporated by reference herein.

Other 3′ and/or 5′ caps amenable to the invention are described in U.S. Provisional Application No. 61/223,665, filed Jul. 7, 2009, contents of which are herein incorporated in their entirety.

The 3′ and/or 5′ ends of an oligonucleotide can also be conjugated to other functional molecular entities such as labeling moieties, e.g., fluorophore (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end with phosphate or phosphate analogs. For example, in some embodiments the 5′ end of the oligonucleotide can be phosphorylated or includes a phosphoryl analog at the 5′ terminus. The 5′-phosphate modifications can include those which are compatible with RISC mediated gene silencing. Modifications at the 5′-terminal end can also be useful in stimulating or inhibiting the immune system of a subject. In some embodiments, the 5′-end of the oligonucleotide comprises the modification

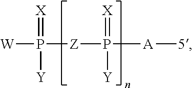

wherein W, X and Y are each independently selected from the group consisting of O, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments n is 1 or 2. It is understood that A is replacing the oxygen linked to 5′ carbon of sugar.

In some embodiments, one or both hydrogen on C5′ of the 5′-terminal nucleotides can be replaced with a halogen, e.g., F.

Exemplary 5′-modificaitons include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)2(X)P—O[—(CH₂)ₐ—P(X)(OH)—O]_{b}-5′, ((HO)2(X)P—[—(CH₂)ₐ—O—P(X)(OH)—O]_{b}-5′; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[(CH₂)ₐ—P(X)(OH)—O]_{b}-5′, Me₂N[—(CH₂)ₐ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

Ligands:

A wide variety of entities, e.g., ligands, can be coupled to the oligonucleotides described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

### SEQ ID NO: 19, AALEALAEALEALAEALEALAEAAAAGGC (GALA);

### SEQ ID NO: 20, AALAEALAEALAEALAEALAEALAAAAGGC (EALA);

### SEQ ID NO: 21, ALEALAEALEALAEA;

### SEQ ID NO: 22, GLFEAIEGFIENGWEGMIWDYG (INF-7);

### SEQ ID NO: 23, GLFGAIAGFIENGWEGMIDGWYG (Inf HA-2);

SEQ ID NO: 24, GLFEAIEGFIENGWEGMIDGWYGCGLFEAIEGFIENGWEGMID GWYGC (diINF-7);
SEQ ID NO: 25, GLFEAIEGFIENGWEGMIDGGCGLFEAIEGFIENGWEGMIDGGC (diINF-3);

### SEQ ID NO: 26, GLFGALAEALAEALAEHLAEALAEALEALAAGGSC (GLF);

### SEQ ID NO: 27, GLFEAIEGFIENGWEGLAEALAEALEALAAGGSC (GALA-INF3);

SEQ ID NO: 28, GLF EAI EGFI ENGW EGnI DG K GLF EAI EGFI ENGW EGnI DG (INF-5, n is norleucine);

### SEQ ID NO: 29, LFEALLELLESLWELLLEA (JTS-1);

SEQ ID NO: 30, GLFKALLKLLKSLWKLLLKA (ppTGl);
SEQ ID NO: 31, GLFRALLRLLRSLWRLLLRA (ppTG20);

### SEQ ID NO: 32, WEAKLAKALAKALAKHLAKALAKALKACEA (KALA);

### SEQ ID NO: 33, GLFFEAIAEFIEGGWEGLIEGC (HA);

### SEQ ID NO: 34, GIGAVLKVLTTGLPALISWIKRKRQQ (Melittin);

### SEQ ID NO: 35, H₅WYG; and

### SEQ ID NO: 36, CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine.

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 2007/0036865; and 2004/0198687, content of all of is incorporated herein by reference in its entirety.

Exemplary cell permeation peptides include, but are not limited to,

SEQ ID NO: 37, RQIKIWFQNRRMKWKK (penetratin);
SEQ ID NO: 38, GRKKRRQRRRPPQC (Tat fragment 48-60);
SEQ ID NO: 39, GALFLGWLGAAGSTMGAWSQPKKKRKV (signal sequence based peptide);

### SEQ ID NO: 40, LLIILRRRIRKQAHAHSK (PVEC);

SEQ ID NO: 41, GWTLNSAGYLLKINLKALAALAKKIL (transportan);
SEQ ID NO: 42, KLALKLALKALKAALKLA (amphiphilic model peptide);

### SEQ ID NO: 43, RRRRRRRRR (Arg9);

SEQ ID NO: 44, KFFKFFKFFK (Bacterial cell wall permeating peptide);

### SEQ ID NO: 45, LLGDFFRKSKEKIGKEFKRIVQRIKDFLRNLVPRTES (LL-37);

SEQ ID NO: 46, SWLSKTAKKLENSAKKRISEGIAIAIQGGPR (cecropin P1);
SEQ ID NO: 47, ACYCRIPACIAGERRYGTCIYQGRLWAFCC (α-defensin)
SEQ ID NO: 48, DHYNCVSSGGQCLYSACPIFTKIQGTCYRGKAKCCK (β-defensin);

### SEQ ID NO: 49, RRRPRPPYLPRPRPPPFFPPRLPPRIPPGFPPRFPPRFPGKR-NH2 (PR-39);

SEQ ID NO: 50, ILPWKWPWWPWRR-NH2 (indolicidin);

### SEQ ID NO: 51, AAVALLPAVLLALLAP (RFGF);

SEQ ID NO: 52, AALLPVLLAAP (RFGF analogue); and
SEQ ID NO: 53, RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactose (GalNAc), multivalent GalNAc, e.g. GalNAc2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 51,410,104; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the oligoncucleotide. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2, 4, 6-triiodophenol and flufenamic acid). Oligonucleotides that comprise a number of phosphorothioate intersugar linkages are also known to bind to serum protein, thus short oligonucleotides, e.g. oligonucleotides of comprising from about 5 to 30 nucleotides (e.g., 5 to 25 nulceotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

Ligands can be coupled to the oligonucleotides at various places, for example, 3′-end, 5′-end, and/or at an internal position. When two or more ligands are present, the ligand can be on opposite ends of an oligonucleotide. In preferred embodiments, the ligand is attached to the oligonucleotides via an intervening tether/linker. The ligand or tethered ligand can be present on a monomer when said monomer is incorporated into the growing strand. In some embodiments, the ligand can be incorporated via coupling to a “precursor” monomer after said “precursor” monomer has been incorporated into the growing strand. For example, a monomer having, e.g., an amino-terminated tether (i.e., having no associated ligand), e.g., monomer-linker-NH₂ can be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of oligonucleotide conjugates include, but are not limited to, U.S. Pat. Nos. 4,828,979; 4,948,882; 5,218, 105; 5,525,465; 5,541,313; 5,545,730; 5,552,538; 5,578, 717, 5,580,731; 5,580,731; 5,591,584; 5,109,124; 5,118, 802; 5,138,045; 5,414,077; 5,486,603; 5,512,439; 5,578, 718; 5,608,046; 4,587,044; 4,605,735; 4,667,025; 4,762, 779; 4,789,737; 4,824,941; 4,835,263; 4,876,335; 4,904, 582; 4,958,013; 5,082,830; 5,112,963; 5,214,136; 5,082, 830; 5,112,963; 5,149,782; 5,214,136; 5,245,022; 5,254, 469; 5,258,506; 5,262,536; 5,272,250; 5,292,873; 5,317,098; 5,371,241, 5,391,723; 5,416,203, 5,451,463; 5,510, 475; 5,512,667; 5,514,785; 5,565,552; 5,567,810; 5,574, 142; 5,585,481; 5,587,371; 5,595,726; 5,597,696; 5,599, 923; 5,599,928; 5,672,662; 5,688,941; 5,714,166; 6,153, 737; 6,172,208; 6,300,319; 6,335,434; 6,335,437; 6,395, 437; 6,444,806; 6,486,308; 6,525,031; 6,528,631; 6,559, 279; content all of which is incorporated by reference in its entirety.

Ligand Carriers:

In some embodiments, the ligands, e.g. endosomolytic ligands, targeting ligands or other ligands, are linked to a monomer which is then incorporated into the growing oligonucleotide strand during chemical synthesis. Such monomers are also referred to as carrier monomers herein. The carrier monomer is a cyclic group or acyclic group; preferably, the cyclic group is selected from the group consisting of pyrrolidinyl, pyrazolinyl, pyrazolidinyl, imidazolinyl, imidazolidinyl, piperidinyl, piperazinyl, [1,3]-dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone. In some embodiments, the cyclic carrier monomer is based on pyrrolidinyl such as 4-hydroxyproline or a derivative thereof.

Exemplary ligands and ligand conjugated monomers amenable to the invention are described in U.S. patent application Ser. No. 10/916,185, filed Aug. 10, 2004; Ser. No. 10/946,873, filed Sep. 21, 2004; Ser. No. 10/985,426, filed Nov. 9, 2004; Ser. No. 10/833,934, filed Aug. 3, 2007; Ser. No. 11/115,989 filed Apr. 27, 2005, Ser. No. 11/119,533, filed Apr. 29, 2005; Ser. No. 11/197,753, filed Aug. 4, 2005; Ser. No. 11/944,227, filed Nov. 21, 2007; Ser. No. 12/328,528, filed Dec. 4, 2008; and Ser. No. 12/328,537, filed Dec. 4, 2008, contents which are herein incorporated in their entireties by reference for all purposes. Ligands and ligand conjugated monomers amenable to the invention are also described in International Application Nos. PCT/US04/001461, filed Jan. 21, 2004; PCT/US04/010586, filed Apr. 5, 2004; PCT/US04/011255, filed Apr. 9, 2005; PCT/US05/014472, filed Apr. 27, 2005; PCT/US05/015305, filed Apr. 29, 2005; PCT/US05/027722, filed Aug. 4, 2005; PCT/US08/061289, filed Apr. 23, 2008; PCT/US08/071576, filed Jul. 30, 2008; PCT/US08/085574, filed Dec. 4, 2008 and PCT/US09/40274, filed Apr. 10, 2009, contents which are herein incorporated in their entireties by reference for all purposes.

Linkers:

In some embodiments, the covalent linkages between the oligonucleotide and other components, e.g. a ligand or a ligand carrying monomer can be mediated by a linker. This linker can be cleavable linker or non-cleavable linker, depending on the application. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the oligonucleotide after transport to the desired target. The intended nature of the conjugation or coupling interaction, or the desired biological effect, will determine the choice of linker group.

As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is, —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

Cleavable Linking Groups:

A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions)

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid celavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleaveable in an acidic environment with a pH of about 6.5 or lower (e.g., about 6.5, 6.0, 5.5, 5.0, or lower), or by agents such as enzymes that can act as a general acid.

### Some Selected Oligonucleotide Modification References

General References:

The oligonucleotides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3, 2′-O-Methyloligoribonucleotides: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

Accordingly, the person skilled in the art is readily in a position to elucidate by means and methods known in the art whether a given compound is a NSD3 inhibitor/antagonist.

Other NSD3 Inhibitors

In some embodiments, a NSD3 inhibitor is a protein inhibitor. For example, a decoy protein or dominant negative protein. In some embodiments, a NSD3i is a decoy protein which is the ET domain of a BET homodomain protein. In some embodiments, a NSD3i is a decoy protein which is the ET domain of a BRD4 of SEQ ID NO: 6, or a homologue or a fragment or homologue thereof.

In some embodiments, a NSD3i inhibitor is a mimetic of the ET domain of BRD4 and comprises the following sequence:

SEEEDKCKPMSYEEKRQLSLDINKLPGEKLGRVVHIIQSREPSLKNSNPDEIEIDFETLKPSTLRELE RYVTSCLRKKRKPQ (SEQ ID NO: 6) or a homologue or fragment of at least 10 amino acids, or at least 15, or at least 20, or at least 25, or at least 30 or at least 35, or at least 40, or at least 45 or at least 50 or more amino acids of:
SEEEDKCKPMSYEEKRQLSLDINKLPGEKLGRVVHIIQSREPSLKNSNPDEIEIDFETLKPSTLRELE RYVTSCLRKKRKPQ (SEQ ID NO: 6). In some embodiments, such a mimetic of the ET domain of BRD4 optionally comprises at least one ectopic mutation, such the mimetic of the ET domain of BRD4 is non-functional and retains its ability to bind to NSD3, but cannot trigger NSD3-BRD4 intracellular signaling.

### Mimetic Inhibitors of NSD3

In some embodiments, the NSD3i inhibitor is a mimetic of the ET domain of BRD4, thus serving as a decoy protein or dominant negative inhibitor of NSD3. In some embodiments, the mimetic comprises SEQ ID NO: 6 or a fragment thereof, and in some embodiments, the mimetic comprises at least one ectopic mutation, whereby the ectopic mutation results in a non-functional ET domain of the BRD4 protein. In some embodiments, the mimetic comprising SEQ ID NO: 6 or a fragment or homologue thereof can bind the NSD3 protein, but not activate BRD4-mediated signaling.

In an alternative embodiment, a NSD3 inhibitor is a non-functional mimetic of NSD3, or soluble NSD3 protein. For example, the NSD3 inhibitor can comprise a non-functional portion of NSD3 which binds to the ET domain of BRD4, thus such non-functional mimetic of NSD3 serves as a competitive inhibitor of NSD3 binding with ET domain of BRD4. In some embodiments, such an NSD3 inhibitor which is a non-functional mimetic of NSD3 comprises SEQ ID NO: 4 or a fragment thereof, wherein the non-functional mimetic comprises at least one ectopic mutation that allows the non-functional mimetic/NSD3 fragment to bind to the ET domain on BRD4, but not activate BRD4-mediated signaling.

Inhibitors to NSD3 also include soluble receptors, or decoy molecules (aka dominant negative inhibitors). As used herein, a “soluble NSD3 protein” is a BRD4-binding NSD3 polypeptide that is non-functional, and in some embodiments, comprises at least SEQ ID NO: 4, that comprises at least on ectopic mutation to result in a non-functional NSD3 protein. Soluble receptors are most commonly receptor polypeptides that comprise at least a portion of the extracellular, ligand binding domain sufficient to bind ligand but lack transmembrane and cytoplasmic domains. Many cell-surface receptors have naturally occurring, soluble counterparts that are produced by proteolysis. Receptor polypeptides are said to be substantially free of transmembrane and intracellular polypeptide segments when they lack sufficient portions of these segments to provide membrane anchoring or signal transduction, respectively. Soluble receptors can comprise additional amino acid residues, such as affinity tags that provide for purification of the polypeptide or provide sites for attachment of the polypeptide to a substrate, or immunoglobulin constant region sequences. Dimeric and higher order multimeric soluble receptors are preferred for their ability to bind ligand with high affinity. A soluble receptor can be prepared as a fusion to a dimerizing protein as disclosed in U.S. Pat. Nos. 5,155,027 and 5,567,584. Dimerizing proteins in this regard include, for example, immunoglobulin fragments comprising constant region and hinge domains (e.g., IgG Fc fragments).

In some embodiments, a NSD3 inhibitor which comprises SEQ ID NO:6 or SEQ ID NO:4 with an ectopic mutation, or fragments thereof, is conjugated to a fusion partner, e.g., a fusion protein which increase the stability of the mimetic protein. In some embodiments, the fusion partner is an IgG1 Fc, e.g., human IgG1 Fc.

In another embodiment, a NSD3 inhibitor which comprises SEQ ID NO:6 or SEQ ID NO:4 with an ectopic mutation, or fragments thereof, is fused to a second fusion partner, such as a carrier molecule to enhance its bioavailability. Such carriers are known in the art and include poly (alkyl) glycol such as poly ethylene glycol (PEG). Fusion to serum albumin can also increase the serum half-life of therapeutic polypeptides.

In some embodiments, a NSD3 inhibitor which comprises SEQ ID NO:6 or SEQ ID NO:4 with an ectopic mutation, or fragments thereof can also be fused to a second fusion partner, for example, to a polypeptide that targets the product to a desired location, or, for example, a tag that facilitates its purification, if so desired. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

In some embodiments, a NSD3 inhibitor which comprises SEQ ID NO:6 or SEQ ID NO:4 with an ectopic mutation, or fragments thereof is modified to achieve adequate circulating half-lives, which impact dosing, drug administration and efficacy. Many approaches have been undertaken with the aim to increase the half-life of biotherapeutics. Small proteins below 60 kD are cleared rapidly by the kidney and therefore do not reach their target. This means that high doses are needed to reach efficacy. Modifications to a NSD3 inhibitor which comprises SEQ ID NO:6, or SEQ ID NO:4 comprising an ectopic mutation, or fragments thereof encompassed in the methods of the present invention to increase the half-life of proteins in circulation include: PEGylation; conjugation or genetic fusion with proteins, e.g., transferrin (WO06096515A2), albumin, growth hormone (US2003104578AA); conjugation with cellulose (Levy and Shoseyov, 2002); conjugation or fusion with Fc fragments; glycosylation and mutagenesis approaches (Carter, 2006), which are incorporated herein by reference.

In the case of PEGylation, polyethylene glycol (PEG) is conjugated to a NSD3 inhibitor which comprises SEQ ID NO:6, or SEQ ID NO:4 comprising an ectopic mutation, or fragments thereof, which can be for example a plasma protein, antibody or antibody fragment. The first studies regarding the effect of PEGylation of antibodies were performed in the 1980s. The conjugation can be done either enzymatically or chemically and is well established in the art (Chapman, 2002; Veronese and Pasut, 2005). With PEGylation the total size can be increased, which reduces the chance of renal filtration. PEGylation further protects from proteolytic degradation and slows the clearance from the blood. Further, it has been reported that PEGylation can reduce immunogenicity and increase solubility. The improved pharmacokinetics by the addition of PEG is due to several different mechanisms: increase in size of the molecule, protection from proteolysis, reduced antigenicity, and the masking of specific sequences from cellular receptors. In the case of antibody fragments (Fab), a 20-fold increase in plasma half-life has been achieved by PEGylation (Chapman, 2002).

To date there are several approved PEGylated drugs, e.g., PEG-interferon alpha2b (PEG-INTRON) marketed in 2000 and alpha2a (Pegasys) marketed in 2002. A PEGylated antibody fragment against TNF alpha, called Cimzia or Certolizumab Pegol, was filed for FDA approval for the treatment of Crohn's disease in 2007 and has been approved on Apr. 22, 2008. A limitation of PEGylation is the difficulty in synthesizing long monodisperse species, especially when PEG chains over 1000 kD are needed. For many applications, polydisperse PEG with a chain length over 10000 kD is used, resulting in a population of conjugates having different length PEG chains, which need extensive analytics to ensure equivalent batches between productions. The different length of the PEG chains may result in different biological activities and therefore different pharmacokinetics. Another limitation of PEGylation is a decrease in affinity or activity as it has been observed with alpha-interferon Pegasys, which has only 7% of the antiviral activity of the native protein, but has improved pharmacokinetics due to the enhanced plasma half-life.

In some embodiments, a NSD3 inhibitor which comprises SEQ ID NO:6, or SEQ ID NO:4 comprising an ectopic mutation, or fragments thereof is conjugated with a long lived protein, e.g. albumin, which is 67 kD and has plasma half-life of 19 days in human (Dennis et al., 2002). Albumin is the most abundant protein in plasma and is involved in plasma pH regulation, but also serves as a carrier of substances in plasma. In the case of CD4, increased plasma half-life has been achieved after fusing it to human serum albumin (Yeh et al., 1992). Other examples for fusion proteins are insulin, human growth hormone, transferrin and cytokines (Ali et al., 1999; Duttaroy et al., 2005; Melder et al., 2005; Osborn et al., 2002a; Osborn et al., 2002b; Sung et al., 2003) and see (US2003104578A1, WO06096515A2, and WO07047504A2, herein incorporated in entirety by reference).

The effect of glycosylation on plasma half-life and protein activity has also been extensively studied. In the case of tissue plasminogen activator (tPA) the addition of new glycosylation sites decreased the plasma clearance, and improved the potency (Keyt et al., 1994). Glycoengineering has been successfully applied for a number of recombinant proteins and immunoglobulins (Elliott et al., 2003; Raju and Scallon, 2007; Sinclair and Elliott, 2005; Umana et al., 1999). Further, glycosylation influences the stability of immunoglobulins (Mimura et al., 2000; Raju and Scallon, 2006).

In some embodiments, a NSD3 inhibitor which comprises SEQ ID NO:6, or SEQ ID NO:4 comprising an ectopic mutation, or fragments thereof can be fused to the Fc fragment of an IgG (Ashkenazi and Chamow, 1997). The Fc fusion approach has been utilized, for example in the Trap Technology developed by Regeneron (e.g. IL1 trap and VEGF trap). The use of albumin to extend the half-life of peptides has been described in US2004001827A1. Positive effects of albumin have also been reported for Fab fragments and scFv-HSA fusion protein (Smith et al., 2001). It has been demonstrated that the prolonged serum half-life of albumin is due to a recycling process mediated by the FcRn (Anderson et al., 2006; Chaudhury et al., 2003; Smith et al., 2001).

In some embodiments, a NSD3 inhibitor which comprises SEQ ID NO:6, or SEQ ID NO:4 comprising an ectopic mutation, or fragments thereof is conjugated to a biotinylated Fc protein, as disclosed in US application 2010/0209424, which is incorporated herein in its entirety by reference.

As used herein, the term “conjugate” or “conjugation” refers to the attachment of two or more entities to form one entity. For example, the methods of the present invention provide conjugation of a peptide comprising SEQ ID NO: 6, or SEQ ID NO: 4 comprising an ectopic mutation (to result in a non-functional NSD3 binding region with ET domain of BRD4) is joined with another entity, for example a moiety such as a first fusion partner that makes the mimetic protein stable, such as Ig carrier particle, for example IgG1 Fc. The attachment can be by means of linkers, chemical modification, peptide linkers, chemical linkers, covalent or non-covalent bonds, or protein fusion or by any means known to one skilled in the art. The joining can be permanent or reversible. In some embodiments, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention.

According to the present invention, a NSD3 inhibitor which comprises SEQ ID NO:6, or SEQ ID NO:4 comprising an ectopic mutation, or fragments thereof, can be linked to the first fusion partner via any suitable means, as known in the art, see for example U.S. Pat. Nos. 4,625,014, 5,057,301 and 5, 514,363, which are incorporated herein in their entirety by reference. For example, a mimetic protein (e.g., a non-functional mimetic of SEQ ID NO: 6 or SEQ ID NO: 4) can be covalently conjugated to the IgG1 Fc, either directly or through one or more linkers. In one embodiment a NSD3 inhibitor which comprises SEQ ID NO:6, or SEQ ID NO:4 comprising an ectopic mutation, or fragments thereof as disclosed herein is conjugated directly to the first fusion partner (e.g. Fc), and in an alternative embodiment, a NSD3 inhibitor which comprises SEQ ID NO:6, or SEQ ID NO:4 comprising an ectopic mutation, or fragments thereof as disclosed herein can be conjugated to a first fusion partner (such as IgG1 Fc) via a linker, e.g. a transport enhancing linker.

A large variety of methods for conjugation of a NSD3 inhibitor which comprises SEQ ID NO:6, or SEQ ID NO:4 comprising an ectopic mutation, or fragments thereof as disclosed herein with a first fusion partner (e.g. Fc) are known in the art. Such methods are e.g. described by Hermanson (1996, Bioconjugate Techniques, Academic Press), in U.S. Pat. No. 6,180,084 and U.S. Pat. No. 6,264,914 which are incorporated herein in their entirety by reference and include e.g. methods used to link haptens to carriers proteins as routinely used in applied immunology (see Harlow and Lane, 1988, “Antibodies: A laboratory manual”, Cold Spring Harbor Laboratory Press, Cold Spring Harbor, N.Y.). It is recognized that, in some cases, a NSD3 inhibitor which comprises SEQ ID NO:6, or SEQ ID NO:4 comprising an ectopic mutation, or fragments thereof can lose efficacy or functionality upon conjugation depending, e.g., on the conjugation procedure or the chemical group utilized therein. However, given the large variety of methods for conjugation the skilled person is able to find a conjugation method that does not or least affects the efficacy or functionality of the mimetic of the ET domain of BRD4 or the NSD3 protein which interacts with the ET domain of BRD4, such that it still binds but retains its non-functional activity.

Suitable methods for conjugation of a NSD3 inhibitor which comprises SEQ ID NO:6, or SEQ ID NO:4 comprising an ectopic mutation, or fragments thereof as disclosed herein with a first fusion partner (e.g. Fc) include e.g. carbodimide conjugation (Bauminger and Wilchek, 1980, Meth. Enzymol. 70: 151-159). Alternatively, a moiety can be coupled to a targeting agent as described by Nagy et al., Proc. Natl. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde crosslinking.

One can use a variety of different linkers to conjugate a NSD3 inhibitor which comprises SEQ ID NO:6, or SEQ ID NO:4 comprising an ectopic mutation, or fragments thereof as disclosed herein with a first fusion partner (e.g. Fc), for example but not limited to aminocaproic horse radish peroxidase (HRP) or a heterobiofunctional cross-linker, e.g. carbonyl reactive and sulfhydryl-reactive cross-linker. Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multi-step protocols can offer a great control of conjugate size and the molar ratio of components.

In some embodiments, a NSD3 inhibitor which comprises SEQ ID NO:6, or SEQ ID NO:4 comprising an ectopic mutation, or fragments thereof useful in the present invention can be modified at their amino termini, for example, so as to increase their hydrophilicity. Increased hydrophobicity enhances exposure of the peptides on the surfaces of lipid-based carriers into which the parent peptide-lipid conjugates have been incorporated. Polar groups suitable for attachment to peptides so as to increase their hydrophilicity are well known, and include, for example and without limitation: acetyl (“Ac”), 3-cyclohexylalanyl (“Cha”), acetyl-serine (“Ac Ser”), acetyl-seryl-serine (“Ac-Ser-Ser-”), succinyl (“Suc”), succinyl-serine (“Suc-Ser”), succinyl-seryl-serine (“Suc-Ser-Ser”), methoxy succinyl (“MeO-Suc”), methoxy succinyl-serine (“MeO-Suc-Ser”), methoxy succinyl-seryl-serine (“MeO-Suc-Ser-Ser”) and seryl-serine (“Ser-Ser-”) groups, polyethylene glycol (“PEG”), polyacrylamide, polyacrylomorpholine, polyvinylpyrrolidine, a polyhydroxyl group and carboxy sugars, e.g., lactobionic, N-acetyl neuraminic and sialic acids, groups. The carboxy groups of these sugars would be linked to the N-terminus of the peptide via an amide linkage. Presently, the preferred N-terminal modification is a methoxy-succinyl modification.

The portion of NSD3 which interacts with BRD4 has been identified herein. Proteolytic processing to remove the portion of the NSD3 molecule that interacts with BRD4 is also encompassed in the present invention, e.g., an agent which removes and/or catalytically cleaves and destroys the portion of the NSD3 protein which binds to the BRD4 protein is encompassed in this present invention.

The following describes other NSD3 inhibitors for use in accordance with the methods and compositions as disclosed herein.

modRNA

In some embodiments, such a NSD3i can be expressed in the cell or delivered by modified RNA (modRNA) by methods known by one of ordinary skill in the art. For example, a decoy molecule NSD3i, such as SEQ ID NO: 6 or a fragment thereof, can be expressed as a modRNA. Synthetic, modified-RNAs as described herein can also be used to express a protein of interest therapeutically in a target tissue or organ by administration of a synthetic, modified-RNA composition to an individual or in alternative embodiments, by contacting cells with a synthetic, modified-RNA ex vivo, and then administering such cells to a subject. In one aspect, cells can be transfected with a modified RNA to express a therapeutic protein using an ex vivo approach in which cells are removed from a patient, transfected by e.g., electroporation or lipofection, and re-introduced to the patient. Continuous or prolonged administration in this manner can be achieved by electroporation of blood cells that are re-infused to the patient.

Synthetic modified RNA's for use in the compositions, methods and kits as disclosed herein are described in U.S. Application 2012/0322865 which is incorporated herein in their entirety by reference.

As used herein, the term “synthetic, modified RNA” (also referred herein as MOD-RNA) refers to a nucleic acid molecule encoding a factor, such as a polypeptide, to be expressed in a host cell, which comprises at least one modified nucleoside and has at least the following characteristics as the term is used herein: (i) it can be generated by in vitro transcription and is not isolated from a cell; (ii) it is translatable in vivo in a mammalian (and preferably human) cell; and (iii) it does not provoke or provokes a significantly reduced innate immune response or interferon response in a cell to which it is introduced or contacted relative to a synthetic, non-modified RNA of the same sequence. A synthetic, modified-RNA as described herein permits repeated transfections in a target cell or tissue in vivo; that is, a cell or cell population transfected in vivo with a synthetic, modified-RNA molecule as described herein tolerates repeated transfection with such synthetic, modified-RNA without significant induction of an innate immune response or interferon response. These three primary criteria for a synthetic, modified RNA molecule described above are described in greater detail below.

First, the synthetic, modified-RNA must be able to be generated by in vitro transcription of a DNA template. Methods for generating templates are well known to those of skill in the art using standard molecular cloning techniques. An additional approach to the assembly of DNA templates that does not rely upon the presence of restriction endonuclease cleavage sites is also described herein (termed “splint-mediated ligation”). The transcribed, synthetic, modified-RNA polymer can be modified further post-transcription, e.g., by adding a cap or other functional group.

To be suitable for in vitro transcription, the modified nucleoside(s) must be recognized as substrates by at least one RNA polymerase enzyme expressed by the tissue or cell which is transfected with the MOD-RNA. Generally, RNA polymerase enzymes can tolerate a range of nucleoside base modifications, at least in part because the naturally occurring G, A, U, and C nucleoside bases differ from each other quite significantly. Thus, the structure of a modified nucleoside base for use in generating the synthetic, modified-RNAs described herein can generally vary more than the sugar-phosphate moieties of the modified nucleoside. That said, ribose and phosphate-modified nucleosides or nucleoside analogs are known in the art that permit transcription by RNA polymerases. In some embodiments of the aspects described herein, the RNA polymerase is a phage RNA polymerase. The modified nucleotides pseudouridine, m5U, s2U, m6A, and m5C are known to be compatible with transcription using phage RNA polymerases, while N1-methylguanosine, N1-methyladenosine, N7-methylguanosine, 2′-)-methyluridine, and 2′-O-methylcytidine are not. Polymerases that accept modified nucleosides are known to those of skill in the art.

It is also contemplated that modified polymerases can be used to generate synthetic, modified-RNAs, as described herein. Thus, for example, a polymerase that tolerates or accepts a particular modified nucleoside as a substrate can be used to generate a synthetic, modified-RNA including that modified nucleoside.

Second, the synthetic, modified-RNA must be translatable in vivo by the translation machinery of a eukaryotic, preferably mammalian, and more preferably, human cell in vivo. Translation in vivo generally requires at least a ribosome binding site, a methionine start codon, and an open reading frame encoding a polypeptide. Preferably, the synthetic, modified-RNA also comprises a 5′ cap, a stop codon, a Kozak sequence, and a polyA tail. In addition, mRNAs in a eukaryotic cell are regulated by degradation, thus a synthetic, modified-RNA as described herein can be further modified to extend its half-life in the cell by incorporating modifications to reduce the rate of RNA degradation (e.g., by increasing serum stability of a synthetic, modified-RNA).

Nucleoside modifications can interfere with translation. To the extent that a given modification interferes with translation, those modifications are not encompassed by the synthetic, modified-RNA as described herein. One can test a synthetic, modified-RNA for its ability to undergo translation and translation efficiency using an in vivo translation assay (e.g., using a MOD-RNA encoding a cre recombinase gene in an in vivo mouse cre model assay, or MOD-RNA encoding luciferase and detecting expression in vivo using a biolumescence assay of the translated protein) and detecting the amount of the polypeptide produced using SDS-PAGE, Western blot, or immunochemistry, biolumescence assays etc. The translation of a synthetic, modified-RNA comprising a candidate modification is compared to the translation of an RNA lacking the candidate modification, such that if the translation of the synthetic, modified-RNA having the candidate modification remains the same or is increased then the candidate modification is contemplated for use with the compositions and methods described herein. It is noted that fluoro-modified nucleosides are generally not translatable and can be used herein as a negative control for an in vitro translation assay.

Third, the synthetic, modified-RNA provokes a reduced (or absent) innate immune response in vivo or reduced interferon response in vivo by the transfected tissue or cell population. mRNA produced in eukaryotic cells, e.g., mammalian or human cells, is heavily modified, the modifications permitting the cell to detect RNA not produced by that cell. The cell responds by shutting down translation or otherwise initiating an innate immune or interferon response. Thus, to the extent that an exogenously added RNA can be modified to mimic the modifications occurring in the endogenous RNAs produced by a target cell, the exogenous RNA can avoid at least part of the target cell's defense against foreign nucleic acids. Thus, in some embodiments, synthetic, modified-RNAs as described herein include in vitro transcribed RNAs including modifications as found in eukaryotic/mammalian/human RNA in vivo. Other modifications that mimic such naturally occurring modifications can also be helpful in producing a synthetic, modified-RNA molecule that will be tolerated by a cell.

Antagomirs:

Antagomirs harbor various modifications for RNAse protection and pharmacologic properties, such as enhanced tissue and cellular uptake. They differ from normal RNA by, for example, complete 2′-O-methylation of sugar, phosphorothioate intersugar linkage and, for example, a cholesterol-moiety at 3′-end. In some embodiments, antagomir comprises a 2′-O-methylmodification at all nucleotides, a cholesterol moiety at 3′-end, two phsophorothioate intersugar linkages at the first two positions at the 5′-end and four phosphorothioate linkages at the 3′-end of the molecule. Antagomirs can be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety.

Ribozymes:

Ribozymes are oligonucleotides having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). At least six basic varieties of naturally-occurring enzymatic RNAs are known presently. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Methods of producing a ribozyme targeted to any target sequence are known in the art. Ribozymes can be designed as described in Int. Pat. Appl. Publ. No. WO 93/23569 and Int. Pat. Appl. Publ. No. WO 94/02595, each specifically incorporated herein by reference, and synthesized to be tested in vitro and in vivo, as described therein.

However, use of NSD3 inhibitors in accordance with the present invention is not limited to known NSD3inhibitors. Accordingly, also yet unknown NSD3 inhibitors may be used in accordance with the present invention. Such inhibitors may be identified by the methods described and provided herein and methods known in the art, like high-throughput screening using biochemical assays for inhibition of NSD3. Assays for screening of potential NSD3inhibitors and, in particular, for identifying selective NSD3 inhibitors as defined herein are described herein. Based on his general knowledge a person skilled in the art is easily in the position to identify inhibitors or verify the inhibiting activity of compounds suspected of being NSD3inhibitors. These tests may be employed on cell(s) or cell culture(s) described in the appended example, but also further cell(s)/tissue(s)/cell culture(s) may be used, such as cell(s)/tissue(s)/cell culture(s) derived from biopsies.

### BET Inhibitors (BETi)

As used herein, the term “BET inhibitor” or “BETi” denotes a compound which inhibits the binding of a bromodomain with its cognate acetylated proteins. In one embodiment the BET inhibitor is a compound which inhibits the binding of a BET protein to acetylated lysine residues. In a further embodiment the BET inhibitor is a compound which inhibits the binding of a BET protein to acetylated lysine residues on histones, particularly histones H3 and H4. In a particular embodiment the BET inhibitor is a compound that inhibits the binding of BET family bromodomains to acetylated lysine residues (hereafter referred to as a “BET family bromodomain inhibitor”). In one embodiment the BET family bromodomain is BRD2, BRD3 or BRD4, in particular BRD2 or BRD3. A BET family bromodomain inhibitor is a compound which has a plC50≧5.0 of at least in one or more of the binding assays described herein, or described in International Patent Application WO2013/026874, which is incorporated herein in its entirety by reference.

In a particular embodiment the BET inhibitor is a compound that inhibits the binding of BET family bromodomains to acetylated lysine residues (hereafter referred to as a “BET family bromodomain inhibitor”). In one embodiment the BET family bromodomain is BRD2, BRD3 or BRD4, in particular BRD2 or BRD3. A BET family bromodomain inhibitor is a compound which has a pIC50≧5.0 of at least in one or more of the binding assays described herein, or described in International Patent Application WO2013/026874, which is incorporated herein in its entirety by reference.

BET inhibitors are well known in the art and include, for example, but are not limited to JQ1, disclosed in WO/2009/084693 and GSK-525762A (also known as I-BET762, Wynce et al., Oncotarget. 2013; 4(12): 2419-2429 Inhibition of BET bromodomain proteins as a therapeutic approach in prostate cancer), LY294002 (Dittmann et al., “The Commonly Used PI3-Kinase Probe LY294002 is an Inhibitor of BET Bromodomains”. ACS Chemical Biology: 2013, 131210150813004.). BET inhibitors are also disclosed in US Application 2012/0208800 and International Applications WO201105484 and WO2006/032470 (SmithKline Beecham Corporation), which are each incorporated herein in their entirety. Such compounds can be prepared by methods described therein.

JQ1, also known as: (S)-tert-butyl 2-(4-(4-chlorophenyl)-2,3,9-trimethyl-6H-thieno[3,2-f][1,2,4]triazolo[4,3-a][1,4]diazepin-6-yl)acetate, and disclosed in WO/2009/084693, has the following structure:

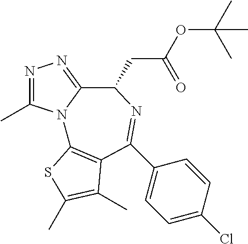 [SMILES: Cc1sc2c(c1C)C(c1ccc(Cl)cc1)=N[C@@H](CC(=O)OC(C)(C)C)c1nnc(C)n1-2]

I-BET-762 (also known as: GSK-525762A) is an inhibitor for BET proteins with IC50 of ˜35 nM, and suppresses the production of proinflammatory proteins by macrophages and blocks acute inflammation, highly selective over other bromodomain-containing proteins. I-BET-762 has the following structure:

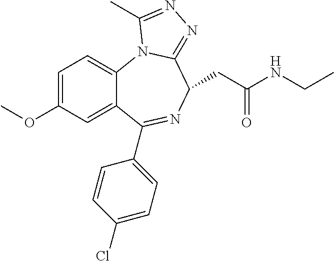 [SMILES: CCNC(=O)C[C@@H]1N=C(c2ccc(Cl)cc2)c2cc(OC)ccc2-n2c(C)nnc21]

LY294002 is the first synthetic molecule known to inhibit PI3Kα/δ/β with IC50 of 0.5 μM/0.57 μM/0.97 μM, respectively; and has been reported to be a BET inhibitor (Dittmann et al., “The Commonly Used PI3-Kinase Probe LY294002 is an Inhibitor of BET Bromodomains”. ACS Chemical Biology: 2013, 131210150813004). LY294002 has the following structure:

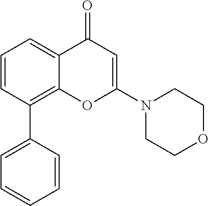 [SMILES: O=c1cc(N2CCOCC2)oc2c(-c3ccccc3)cccc12]

In a further embodiment a BET inhibitor is a compound that is generically or specifically disclosed in PCT publication WO2009/084693 (Mitsubishi Tanabe). Such compounds can be prepared by methods described therein. In a further embodiment the BET inhibitor is 1-[2-(1/-/-benzimidazol-2-ylthio)ethyl]-1,3-dihydro-3-methyl-2H-benzinidazole-2-thione as described in Japanese patent application JP2008-15631 1, which is incorporated herein in its entirety. It will be appreciated that a BET inhibitor used in the present invention may be in the form of a pharmaceutically acceptable salt, solvate (e.g. a hydrate) or prodrug or any other derivative of such a compound which upon administration to the recipient is capable of providing (directly or indirectly) the BET inhibitor of the invention, or an active metabolite or residue thereof. Suitable pharmaceutically acceptable salts can include acid or base addition salts. For a review on suitable salts see Berge et al., J. Pharm. Sci., 66:1-19, (1977). Typically, a pharmaceutically acceptable salt may be readily prepared by using a desired acid or base as appropriate. The resultant salt may precipitate from solution and be collected by filtration or may be recovered by evaporation of the solvent. Suitable prodrugs are recognizable to those skilled in the art, without undue experimentation.

In one embodiment the BET inhibitor is a compound being a small molecule, in particular having a molecular weight of less 750, more particularly less than 500. In one embodiment the BET inhibitor is a compound selected from the group consisting the compounds shown in Table 1.

TABLE 1
Additional Exemplary BET inhibitors

| BET inhibitor | Structure |
|---|---|
| 1-methylethyl ((2S,4R)-1-acetyl-2-methyl-6-{4- [(methylamino)methyl]phenyl}-1,2,3,4-tetrahydro- 4-quinolinyl)carbamate | 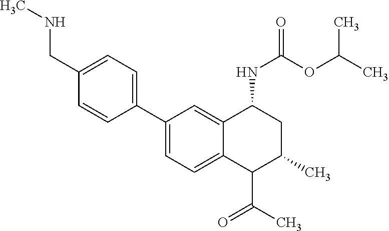 [SMILES: CNCc1ccc(-c2ccc3c(c2)[C@H](NC(=O)OC(C)C)C[C@H](C)C3C(C)=O)cc1] |
| 2-[(4S)-6-(4-Chlorophenyl)-1-methyl-8- (methyloxy)-4H-[1,2,4]triazolo[4,3- a][1,4]benzodiazepin-4-yl]-N-ethylacetamide | 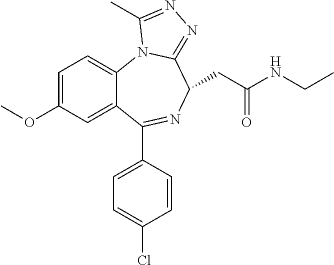 [SMILES: CCNC(=O)C[C@@H]1N=C(c2ccc(Cl)cc2)c2cc(OC)ccc2-n2c(C)nnc21] |
| 7-(3,5-dimethyl-4-isoxazolyl)-8-(methoxy)-1- [(1R)-1-(2-pyridinyl)ethyl]-1,3-dihydro-2H- imidazo[4,5-c]quinolin-2-one | 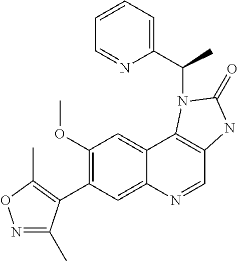 [SMILES: COc1cc2c(cc1-c1c(C)noc1C)ncc1[nH]c(=O)n([C@H](C)c3ccccn3)c12] |
| 7-(3,5-dimethyl-4-isoxazolyl)-8-(methoxy)-1- [(1R)-phenethyl]-2-(tetrahydro-2H-pyran-4-yl)- 1H-imidazo[4,5-c]quinoline | 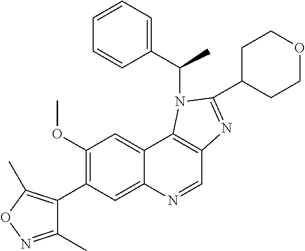 [SMILES: COc1cc2c(cc1-c1c(C)noc1C)ncc1nc(C3CCOCC3)n([C@H](C)c3ccccc3)c12] |
| 4-{(2S,4R)-1-acetyl-4-[(4-chlorophenyl)amino]-2- methyl-1,2,3,4-tetrahydro-6-quinolinyl}benzoic acid | 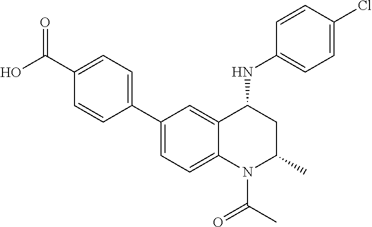 [SMILES: CC(=O)N1c2ccc(-c3ccc(C(=O)O)cc3)cc2[C@H](Nc2ccc(Cl)cc2)C[C@@H]1C] |
| N-{1-methyl-7-[4-(1- piperidinylmethyl)phenyl][1,2,4]triazolo[4,3- a]quinolin-4-yl}urea | 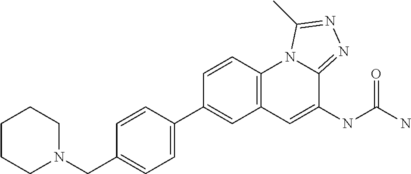 [SMILES: Cc1nnc2c(NC(N)=O)cc3cc(-c4ccc(CN5CCCCC5)cc4)ccc3n12] |

However, use of BET inhibitors in accordance with the present invention is not limited to known NSD3inhibitors. Accordingly, also yet unknown BET inhibitors may be used in accordance with the present invention. Such inhibitors may be identified by the methods described and provided herein and methods known in the art, like high-throughput screening using biochemical assays for inhibition of BET. Assays for screening of potential BET inhibitors and, in particular, for identifying selective BET inhibitors as defined herein are described herein. Based on his general knowledge a person skilled in the art is easily in the position to identify inhibitors or verify the inhibiting activity of compounds suspected of being NSD3inhibitors. These tests may be employed on cell(s) or cell culture(s) described in the appended example, but also further cell(s)/tissue(s)/cell culture(s) may be used, such as cell(s)/tissue(s)/cell culture(s) derived from biopsies.

### Method of Treatment of a Subject

The present invention relates generally to a method of treating a proliferative disease or disorder in a subject, where the proliferative disease or disorder is a cancer, e.g., NMC or a NSD3-dependent cancer, including NSD3/NUT, BRD4/NUT and BRD3/NUT cancers, as well as acute myeloid leukemia or myelodystoplastic syndrome with NUP98-NSD3 fusion gene. For example, an effective amount of compound of a NSD3i or BETi as disclosed herein, or analogue or derivative thereof is administered to a subject with a NSD3/NUT cancer or a NSD3i is administered to a BRD4/NUT or BRD3/NUT cancer. Thus, by using the methods of the present invention, one can intervene in the proliferative disease, for example cancer, ameliorate the symptoms, and in some cases cure the disease.

In a related embodiment, the invention contemplates the practice of the method in conjunction with other therapies such as conventional chemotherapy directed against solid tumors and for control of establishment of metastases. The administration of the compounds described herein is typically conducted prior to and/or at the same time and/or after chemotherapy, although it is also encompassed within the present invention to inhibit cell proliferation after a regimen of chemotherapy at times where the tumor tissue will be responding to the toxic assault by inducing angiogenesis to recover by the provision of a blood supply and nutrients to the tumor tissue. In addition, the pharmaceutical compositions of the invention for the treatment of proliferative disorders, for example cancer, can be administrated prophylactically and/or before the development of a tumor, if the subject has been identified as to have a risk of developing cancer, for example to subjects that are positive for biomarkers of cancer cells or tumors. Insofar as the present methods apply to inhibition of cell proliferation, the methods can also apply to inhibition of tumor tissue growth, to inhibition of tumor metastases formation, and to regression of established tumors.

The inventive methods disclosed herein provide for the parenteral and oral administration of the compounds of the present invention, e.g., NSD3i and/or BETi as disclosed herein in combination with other pharmaceutical compositions to subjects in need of such treatment. Parenteral administration includes, but is not limited to, intravenous (IV), intramuscular (IM), subcutaneous (SC), intraperitoneal (IP), intranasal, and inhalant routes. In the method of the present invention, the NSD3i and/or BETi are preferably administered orally. IV, IM, SC, and IP administration may be by bolus or infusion, and may also be by slow release implantable device, including, but not limited to pumps, slow release formulations, and mechanical devices. The formulation, route and method of administration, and dosage will depend on the disorder to be treated and the medical history of the subject. In general, a dose that is administered by subcutaneous injection will be greater than the therapeutically-equivalent dose given intravenously or intramuscularly. Preferably, the dose of a NSD3i and/or BETi as disclosed herein will be administered at doses from about 0.1 mg to about 250 mg of body weight. In some embodiments, the dose of compounds of the present invention will be from about 1 mg to about 60 mg.

The methods of the present invention for treating cancer, e.g., NMC or a NSD3-dependent cancer, including NSD3/NUT, BRD4/NUT and BRD3/NUT cancers, comprising contacting a tissue in which proliferation is occurring, or is at risk for occurring, with the compositions of the present invention comprising a therapeutically effective amount of a NSD3i and/or BETi as disclosed herein or functional derivatives thereof.

In some embodiments, the subject treated by the methods of the present invention in its many embodiments is a human subject, although it is to be understood that the principles of the invention indicate that the invention is effective with respect to all mammals. In this context, a mammal is understood to include any mammalian species in which treatment of diseases associated with cancer or a proliferative-related disorder is desirable, particularly agricultural and domestic mammalian species, as well as transgenic animals.

### Subjects Amenable to Treatment

The following explanations refer, to rearrangements in the NUT gene which is characteristic especially for NUT midline carcinoma, and identifying and selecting subjects for treatment.

As discussed herein, subjects with a NUT translocation of NSD3/NUT, BRD4/NUT or BRD3/NUT can be treated with NSD3i as disclosed herein. That is, a subject with NUT fused to any of, or a combination of, NSD3, or BRD4 or BRD3 are amenable to treating with a NSD3i as disclosed herein. In alternative embodiments, subjects with NSD3/NUT translocations can be treated with BETi as disclosed herein, that is, a subject with NUT fused to NSD3 can be treated with a BETi as disclosed herein.

The below explanations concerning the NUT gene and NUT protein, apply, mutatis mutandis, to other nucleic acid sequences and amino acid sequences to be employed in context of the present invention, such as partner genes of NUT in NUT fusion genes like NSD3/NUT fusion genes, BRD4/NUT fusion genes, BRD3/NUT fusion genes or NUT-variant fusion genes characteristic for NMC.

The term “NUT gene” (“nuclear protein in testis”) as used in context of this invention refers to a gene encoding an unstructured polypeptide of unknown function that is highly expressed in normal spermatids; see Schwartz. It has been reported that the NUT protein binds to the histone acetyltransferase (HAT) p300; see Schwartz. The nucleic acid sequence of the human NUT gene and the corresponding amino acid sequence is shown in SEQ ID NO: 7 (accession number NM_175741.1), where the coding region ranges from nucleotides 156 to 3554, which encodes amino acids sequence of SEQ ID NO: 8. Generally, the term NUT used herein refers to any amino acid sequence having (partial) NUT activity as described herein and nucleic acid sequence(s) encoding such (an) amino acid sequence(s).

Accordingly, the explanations apply, mutatis mutandis, to members of the BET family (BRD2, BRDT and in particular, human BRD4 gene and BRD4 protein (encoded by nucleic acid sequence of SEQ ID NOs: 9 (accession number NM_058243.2) where the coding region ranges from nucleotide 223 to 4311 of SEQ ID NO: 9, which encodes the amino acid sequence of SEQ ID NO:10) and human BRD3 gene and BRD3 protein (encoded by nucleic acid sequence of SEQ ID NO: 11 (NM_007371.3), where the coding region is from nucleotide 189 to 2369, which encodes amino acid sequence of SEQ ID NO: 12).

The term “rearrangement in the NUT gene” used herein refers to any rearrangement in the NUT gene that is characteristic for NUT midline carcinoma (NMC). Exemplary “rearrangements in the NUT gene” as well as methods for their detection are known in the art (see, for example, French (2010) J Clin Pathol,). For example, the rearrangement can be or can be caused by a translocation of the NUT gene (or a part or fragment thereof).

As disclosed herein, the inventors have discovered the rearrangement of the NUT gene resulting in the formation of a NSD3/NUT fusion gene. Treatment of subjects with such NSD3/NUT fusion genes with NSD3i and/or BETi is encompassed herein. Also envisaged in this context is the formation of a fusion gene comprising a sequence encoding the complete NSD3 gene, and/or one or more parts or fragments thereof as disclosed herein, and also comprising a sequence encoding the complete NUT gene and/or one or more parts or fragments thereof. The exemplary NSD3-NUT fusion protein is composed of the N-terminal of NSD3 (amino acids 1-569) and almost the entire protein sequence of NUT (amino acids 8-1132). The N-terminal of NSD3 includes a domain which interacts with the ET domain on BRD4 and other bromodomain BET proteins, such as BRD1, BRD2 and BRD3.

Additionally, another NUT translocation are known in the art, including the tl 5; 19 translocation, which results in the formation of the BRD4-NUT fusion gene. Accordingly, treatment of subjects with a NSD3i having a rearrangement in the NUT gene which are, or are caused/associated by, the formation of a BRD4/NUT fusion gene are encompassed in this invention as disclosed herein. Also envisaged in this context is the formation of a fusion gene comprising a sequence encoding the complete BRD4 gene, and/or one or more parts or fragments thereof and comprising a sequence encoding the complete NUT gene and/or one or more parts or fragments thereof. The exemplary BRD4-NUT fusion protein is composed of the N-terminal of BRD4 (amino acids 1-720 out of 1372) and almost the entire protein sequence of NUT (amino acids 6-1 127). The N-terminal of BRD4 includes bromodomains 1 and 2 and other, less well characterized functional domains.

In some embodiments, subjects with NMC with other rearrangements in the NUT gene can also be treated with NSD3i inhibitor as disclosed herein. For example, subjects with the NMC subtype resulting from the formation of the BRD3-NUT fusion gene. Again, the formation of a fusion gene comprising a sequence encoding the complete BRD3 gene, and/or one or more parts or fragments thereof and comprising a sequence encoding the complete NUT gene and/or one or more parts or fragments thereof is envisaged herein.

The rearrangements in the NUT gene and optionally mutations/rearrangements/aberrant expression of further genes can be detected by methods known in the art. Such methods are, for example described in French CA, 2010 (NUT midline carcinoma. French CA. Cancer Genet Cytogenet. 2010 November; 203(1): 16-20.). A person skilled in the art is in the position to adapt the methods for detecting rearrangements in genes described in the above-mentioned documents to the rearrangements in the NUT gene described herein and further rearrangements in this gene known in the art. A person skilled in the art will readily understand that also rearrangements in said gene not described herein but known in the art or mutations yet to be identified may also be used in the context of the present invention. Exemplary, non-limiting methods to be used in the detection of rearrangements in the NUT gene are described in WO 2010/011700, Haack (2009), French (2010) Cancer Genet Cytogenet and French (2010) J Clin Pathol.

Particularly preferred is diagnosis via in situ hybridization (FISH, CISH, SISH and the like), since these methods can detect any rearrangement in the NUT gene. However, also detection of a gene product of the above described NUT fusion genes is envisaged using routine techniques like immunohistochemical methods, Northern Blot, Real time PCR and the like. This especially useful in cases where said rearrangement in the NUT gene is reflected in expression of the formed NUT fusion gene, as the expression level of the formed NUT fusion gene may be detected. Such methods are particularly envisaged in the detection of NSD3/NUT or BRD3/NUT transcripts and/or BRD4/NUT transcripts. Also immunohistochemical methods (or other routine methods like Western Blots etc.) may be employed to detect expression products on a protein level. For example, antibodies French (2010) Cancer Genet Cytogenet or Haack (2009). describe the use of a diagnostic NUT specific monoclonal antibody, taking advantage of the fact the native protein is not expressed outside of the testis. Further methods which are useful for detecting mutations or rearrangements are methods for sequencing of nucleic acids (e.g. Sanger di-deoxy sequencing), “next generation” methods, single molecule sequencing, methods enabling detection of variant alleles/mutations, such as Real-time PCR, PCR-RFLP assay (see Cancer Research 59 (1999), 5169-5175), mass-spectrometric genotyping (e.g. MALDI-TOF), HPLC, enzymatic methods and SSPC (single strand conformation polymorphism analysis; see Pathol Int (1996) 46, 801-804).

In particular, such methods may include enzymatic amplification of DNA or cDNA fragments using oligonucleotides specifically hybridizing to exonic or intronic parts of the rearranged NUT gene by PCT. Such amplifications may be carried out in two reactions when employing, genomic DNA or even in only a single reaction when employing cDNA. The resulting PCR products may be subjected to either conventional Sanger-based dideoxy nucleotide sequencing methods or employing novel parallel sequencing methods (“next generation sequencing”) such as those marketed by Roche (454 technology), Illumina (Solexa technology) or ABI (Solid technology). Rearrangements or mutations may be identified from sequence reads by comparison with publicly available gene sequence data bases. Alternatively, mutations may be identified by allele-specific incorporation of probes that can either be detected using enzymatic detection reactions, fluorescence, mass spectrometry or others; see Vogeser (2007) Dtsch Arztebl 104 (31-32), A2194-200. Paraffin-embedded clinical material may be used in the detection of rearrangements in the NUT gene. Detection may comprise a histolopathology review of the sample to be tested to ensure tumor tissue is present. A commercially available Kit to be used in the detection method is the AllPrep DNA/RNA FFPE Kit form Quiagen (Germany). Further kits to be used for detecting rearrangements in the NUT gene are commercially available. A positive result in the detection method indicates the presence of (a) rearrangement(s) in the NUT gene.

As mentioned above, (a) tumor cell(s)/tumor(s) with (a) rearrangement(s) in the NUT gene, e.g., NSD3/NUT, BRD4/NUT and/or BRD3/NUT (are) sensitive to treatment with selective NSD3i inhibitors. Furthermore, a tumor with the rearrangement of the NUT gene resulting in NSD3/NUT fusion gene is sensitive to treatment with BET inhibitors as disclosed herein. Therefore, it is envisaged that (a) tumor cell(s)/tumor(s) with (a) with (a) rearrangement! s) in the NUT gene might be particularly sensitive to treatment with NSD3 inhibitors. Accordingly, treatment of patients with a selective NSD3i inhibitor (the patients suffering from NMC) may be particularly successful in respect of, for example, prognosis or survival rate.

### Administration of Pharmaceutical Compositions

A physician or veterinarian having ordinary skill in the art can readily determine and prescribe the effective amount of the pharmaceutical composition of a NSD3i and/or BETi as disclosed herein which is required. For example, the physician or veterinarian could start doses of the compounds of the invention employed in the pharmaceutical composition at levels lower than that required in order to achieve the desired therapeutic effect and gradually increase the dosage until the desired effect is achieved.

After formulation of a NSD3i and/or BETi as disclosed herein with an appropriate pharmaceutically acceptable carrier in a desired dosage, the pharmaceutical compositions of this invention can be administered to a subject. The pharmaceutical compositions as disclosed herein can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes.

The phrases “parenteral administration” and “administered parentally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systematically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

When the compounds of the present invention, for example a NSD3i and/or BETi as disclosed herein are administered as pharmaceuticals, to humans and mammals, they can be given per se or as a pharmaceutical composition containing, for example, 0.1 to 99.5% (more preferably, 0.5 to 90%) of active ingredient, i.e., at least one compound of NSD3i and/or BETi as disclosed herein and/or derivative thereof, in combination with a pharmaceutically acceptable carrier.

In general, a suitable daily dose of a NSD3i and/or BETi as disclosed herein will be that amount of the compound which is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.1 mg to about 250 mg per kilogram of body weight per day, more preferably from about 1 mg to about 60 mg per kg per day.

If desired, the effective daily dose of the active compound may be administered as two, three, four, five, six or more sub-doses administered separately at appropriate intervals throughout the day, optionally, in unit dosage forms.

Pharmaceutical compositions comprising a NSD3i and/or BETi as disclosed herein can include a “therapeutically effective amount” or a “prophylactically effective amount” of one or more of the compounds of the present invention, or functional derivatives thereof. An “effective amount” is the amount as defined herein in the definition section and refers to an amount effective, at dosages and for periods of time necessary, to achieve the desired therapeutic result, e.g., a diminishment or prevention of effects associated with proliferative disease states or conditions, such as cancer, for example, NMC or a NSD3-dependent cancer, including NSD3/NUT, BRD4/NUT and BRD3/NUT cancers, A therapeutically effective amount of a NSD3i or BETi as disclosed herein or functional derivatives thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to, or at an earlier stage of disease, the prophylactically effective amount may be less than the therapeutically effective amount. A prophylactically or therapeutically effective amount is also one in which any toxic or detrimental effects of the compound are outweighed by the beneficial effects.

The term “synergy” or “synergistic” as used herein, refers to the interaction of two or more agents so that their combined effect is greater than each of their individual effects at the same dose alone.

Dosage regimens may be adjusted to provide the optimum desired response (e.g. a therapeutic or prophylactic response). For example, a single bolus may be administered, several divided doses may be administered over time or the dose may be proportionally reduced or increased as indicated by the exigency of the therapeutic situation. It is especially advantageous to formulate parenteral compositions in dosage unit form for ease of administration and uniformity of dosage. Actual dosage levels of the active ingredients in the pharmaceutical compositions comprising one or more NSD3i and/or BETi may be varied so as to obtain an amount of the active ingredient which is effective to achieve the desired therapeutic response for a particular subject, composition, and mode of administration, without being toxic to the patient.

The term “dosage unit” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound, a NSD3i and/or BETi and/or derivative thereof and the particular therapeutic or prophylactic effect to be achieved, and (b) the limitations inherent in the art of compounding such an active compound for the treatment of sensitivity in individuals.

In some embodiments, therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount of a NSD3i and/or BETi as disclosed herein is sufficient to reduce or inhibit cell proliferation in a subject suffering from a proliferative disorder, for example cancer, for example, NMC or a NSD3-dependent cancer, including NSD3/NUT, BRD4/NUT and BRD3/NUT cancers, or BRD-dependent, NUT-independent cancers. In some embodiments, the therapeutically effective amount is sufficient to eliminate the proliferative cells, for example eliminate the cancer cells and/or tumor in a subject suffering cancer and/or a proliferative disease.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of a NSD3i and/or BETi as disclosed herein or functional derivatives thereof, and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more NSD3i and/or BETi, including but not limited to such as NSD3i and/or BETi as disclosed herein or functional derivatives thereof are optionally tested in one or more appropriate in vitro and/or in vivo animal models of disease, such as models of cancer, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of a NSD3i and/or BETi as disclosed herein or functional derivatives thereof at various concentrations, e.g., as applied to the mass and overall health of the patient. Administration can be accomplished via single or divided doses.

In vitro models can be used to determine the effective doses of a NSD3i and/or BETi as disclosed herein or functional derivatives thereof as a potential cancer treatment. Suitable in vitro models include, but are not limited to, proliferation assays of cultured tumor cells, growth of cultured tumor cells in soft agar (see Freshney, (1987) Culture of Animal Cells: A Manual of Basic Technique, Wily-Liss, New York, N.Y. Ch 18 and Ch 21), tumor systems in nude mice as described in Giovanella et al., I J. Natl. Can. Inst., 52: 921-30 (1974), mobility and invasive potential of tumor cells in Boyden Chamber assays as described in Pilkington et al., Anticancer Res., 17: 4107-9 (1997), and angiogenesis assays such as induction of vascularization of the chick chorioallantoic membrane or induction of vascular endothelial cell migration as described in Ribatta et al., Intl. J. Dev. Biol., 40: 1189-97 (1999) and Li et al., Clin. Exp. Metastasis, 17:423-9 (1999), respectively. Suitable tumor cells lines are available, e.g. from American Type Tissue Culture Collection catalogs.

In vivo models are the preferred models to determine an effective dose of a NSD3i and/or BETi as disclosed herein or functional derivatives thereof as disclosed herein as potential cancer treatments. Suitable in vivo models include, but are not limited to, mice that carry a mutation in the KRAS oncogene (Lox-Stop-Lox K-RasGi2D mutants, Kras24TYj) available from the National Cancer Institute (NCI) Frederick Mouse Repository. Other mouse models known in the art and that are available include but are not limited to models for breast cancer, gastrointestinal cancer, hematopoietic cancer, lung cancer, mammary gland cancer, nervous system cancer, ovarian cancer, prostate cancer, skin cancer, cervical cancer, oral cancer, and sarcoma cancer (see http://emice.nci.nih.gov/mouse_models/).

In determining the effective amount of a NSD3i and/or BETi as disclosed herein or a functional derivative thereof to be administered in the treatment or prophylaxis of disease the physician evaluates circulating plasma levels, formulation toxicities, and progression of the disease.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. In some embodiments, the compounds of the present invention have an ED₅₀ value ranging from 0.01-10 μM in an assay for inhibition of at NSD3 or a BET family protein, e.g., BRD4.

These compounds may be administered to humans and other animals for therapy by any suitable route of administration, including orally, nasally, as by, for example, a spray, rectally, intravaginally, parentally, intracisternally and topically, as by powders, ointments or drops, including buccally and sublingually.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

An exemplary, non-limiting range for a therapeutically or prophylactically effective amount of a compound of the present invention, for example a NSD3i and/or BETi as disclosed herein and/or functional derivatives thereof of the invention is 0.1-250 mg/kg, and in some embodiments, the dosage is 1-60 mg/kg. In some embodiments, the dose of a NSD3i and/or BETi as disclosed herein is between 30-600 mg/kg/day, or between about 10-1000 mg/kg/day, or between about 50-500 mg/kg/day, or between about 100-100 mg/kg/day, or between about 30-100 mg/kg/day. In some embodiments, the dose is about 30 mg/kg/day. In some embodiments, the dose is about 600 mg/kg/day. In some embodiments, the human equivalent dose (HED) of a NSD3i or BETi (which, if used in mice at 30 mg/kg/day and 600 mg/kg/day) is between about between 2.5-5 mg/kg/day, or between about 1-10 mg/kg/day, or between about 2-5 mg/kg/day, or between about 1-2.5 mg/kg/day, or between about 2-7.5 mg/kg/day. In some embodiments, the dose is about 2.5 mg/kg/day. In some embodiments, the dose is about 5 mg/kg/day.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The invention features an article of manufacture that contains packaging material and compounds of the present invention, for example a NSD3i and/or BETi as disclosed herein and/or functional derivatives thereof in a formulation contained within the packaging material. In some embodiments, a formulation can contain at least one of the compounds of the present invention, for example at least one NSD3i and/or BETi as disclosed herein and/or functional derivatives thereof and the packaging material contains a label or package insert indicating that the formulation can be administered to the subject to treat one or more conditions as described herein, in an amount, at a frequency, and for a duration effective to treat or prevent such condition(s). Such conditions are mentioned throughout the specification and are incorporated herein by reference.

More specifically, the invention features an article of manufacture that contains packaging material and at least one of the compounds of the present invention, for example at least one NSD3i and/or BETi as disclosed herein or a functional derivative thereof contained within the packaging material. The packaging material contains a label or package insert indicating that the formulation can be administered to the subject to alleviate a proliferative disorder, for example cancer in an amount, at a frequency, and for a duration effective treat or prevent symptoms associated with such disease states or conditions discussed throughout this specification.

### Pharmaceutical Compositions

In another embodiment of the invention, a pharmaceutical composition can contain one or more compounds as disclosed, e.g., a NSD3i and/or BETi as disclosed herein. For purpose of administration, a NSD3i and/or BETi as disclosed herein is preferably formulated as a pharmaceutical composition. Pharmaceutical compositions of the present invention comprise a compound of this invention and a pharmaceutically acceptable carrier, wherein the compound is present in the composition in an amount which is effective to treat the condition of interest. Preferably, a pharmaceutical composition of the present invention can include a NSD3i and/or BETi as disclosed herein in an amount from 0.1 mg to 250 mg per dosage depending upon the route of administration, and more typically from 1 mg to 60 mg. Appropriate concentrations and dosages can be readily determined by one skilled in the art.

Pharmaceutically acceptable carriers are familiar to those skilled in the art. For compositions formulated as liquid solutions, acceptable carriers include saline and sterile water, and may optionally include antioxidants, buffers, bacteriostats and other common additives. The compositions can also be formulated as pills, capsules, granules, or tablets which contain, in addition to a compound of this invention, diluents, dispersing and surface active agents, binders, and lubricants. One skilled in this art may further formulate the compounds of this invention in an appropriate manner, and in accordance with accepted practices, such as those disclosed in Remington's Pharmaceutical Sciences, Gennaro, Ed., Mack Publishing Co., Easton, Pa. 1990.

While it is possible for compounds of the present invention, for example a NSD3i and/or BETi as disclosed herein and/or functional derivatives thereof, to be administered alone, it is preferable to administer the compound as a pharmaceutical composition.

Formulations of the invention can be prepared by a number or means known to persons skilled in the art. In some embodiments the formulations can be prepared by combining (i) at least a NSD3i and/or BETi as disclosed herein and/or functional derivatives thereof in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, that comprise one or more of the compounds of the present invention, and/or their functional derivatives thereof for oral or subcutaneous administration.

In certain embodiments, the compounds of the present invention, for example a NSD3i and/or BETi as disclosed herein or functional derivatives thereof are administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are anti-cancer agents. In some embodiments, the therapeutic agents are chemotherapeutic agents, for example but not limited to, cisplatin, paxicital etc. In some embodiments, the therapeutic agents are radiotherapeutic agents.

In some embodiments the pharmaceutical composition comprises compounds of the present invention, for example a NSD3i and/or BETi as disclosed herein and/or functional derivatives thereof, alone or in any plurality of combinations. In other embodiments, the pharmaceutical compositions optionally further comprise one or more additional therapeutic agents including but not limited to chemotherapeutic agents. Examples of chemotherapeutic agents in the pharmaceutical compositions of this invention are, for example but not limited to paclitaxel, cisplatin, doxorubicin and paclitaxel, vermurafib, nitrogen mustards such as cyclophosphamide, ifosfamide, and melphalan; ethylenimines and methylmelamines such as hexamethylmelamine and thiotepa; pyrimidine analogs such as fluorouracil and fluorodeoxyuridine; vinca alkaloids such as vinblastine; epipodophyllotoxins such as etoposide and teniposide; antibiotics such as actinomycin D, doxorubicin, bleomycin, and mithramycin; biological response modifiers such as interferon, platinum coordination complexes such as cisplatin and carboplatin; estrogens such as diethylstilbestrol and ethinyl estradiol; antiandrogens such as flutamine; and gonadotropin releasing hormone analogs such as leuprolide. Other compounds such as decarbazine, nitrosoureas, methotrexate, diticene, and procarbazine are also effective and encompassed for use in the methods of the present invention.

Patient(s) may also be subject to co-therapy/co-treatment with a NSD3i and/or BETi and a further compound/drug (e.g. (a) NUT inhibitors)). Of course, co-therapy/combination therapy to be used in context of the present invention may also comprise radiation therapy, conventional chemotherapy and the like.

The following relates to pharmaceutical compositions and drug combinations. In one embodiment, the present invention relates to a NSD3i and/or BETi as defined herein for use in treating, ameliorating and/or preventing midline carcinoma, like NUT midline carcinoma (NMC). Accordingly, also the use of a NSD3i and/or BETi for the preparation of a pharmaceutical composition for the treatment, amelioration and/or prevention of midline carcinoma, like NUT midline carcinoma (NMC), is envisaged in context of the present invention.

In accordance with the above, the present invention relates to drug combinations and pharmaceutical compositions comprising at least one NSD3i and/or BETi as active ingredient together with at least one pharmaceutically acceptable carrier, excipient and/or diluent and optionally together with one or more other anti-tumor agents As used herein the term “drug combination” refers to a combination of at least to pharmaceutically active agents or therapeutic agents with or without further ingredients, carrier, diluents and/or solvents. As used herein the term “pharmaceutical composition” refers to a galenic formulation of at least one pharmaceutically active agent together with at least one further ingredient, carrier, diluent and/or solvent.

NSD3i and/or BETi may be administered as the sole pharmaceutical agent or in combination with one or more additional therapeutic agents, wherein the drug combination causes no unacceptable adverse effects. This combination therapy includes administration of a single pharmaceutical dosage formulation, which contains a NSD3i and/or BETi and one or more additional therapeutic agents in form of a single pharmaceutical composition, as well as administration of a NSD3i and/or BETi and each additional therapeutic agent in its own separate pharmaceutical dosage formulation, i.e. in its own separate pharmaceutical composition. For example, a NSD3i and/or BETi and a therapeutic agent may be administered to the patient together in a single oral dosage composition such as a tablet or capsule, or each agent may be administered in separate pharmaceutical compositions. Where separate pharmaceutical compositions are used, a NSD3i and/or BETi and one or more additional therapeutic agents may be administered at essentially the same time (e.g., concurrently) or at separately staggered times (e.g., sequentially).

In particular, the NSD3i and/or BETi to be used in accordance with the present invention may be used in fixed or separate pharmaceutical compositions with other anti-tumor agents such as alkylating agents, anti-metabolites, plant-derived anti-tumor agents, hormonal therapy agents, topoisomerase inhibitors, camptothecin derivatives, kinase inhibitors, targeted drugs, antibodies, interferons and/or biological response modifiers, anti-angiogenic compounds, and other antitumor drugs. In this regard, the following is a non-limiting list of examples of secondary agents that may be used in combination with the NSD3 and/or BET inhibitors:
Alkylating agents include, but are not limited to, nitrogen mustard N-oxide, cyclophosphamide, ifosfamide, thiotepa, ranimustine, nimustine, temozolomide, altretamine, apazi-quone, brostallicin. bendamustine, carmustine, estramustine, fotemustine, glufosfamide, mafosfamide, and mitolactol; platinum-coordinated alkylating compounds include, but are not limited to, cisplatin, carboplatin, eptaplatin, lobaplatin, nedaplatin, oxaliplatin, and satraplatin;
Anti-metabolites include, but are not limited to, methotrexate, 6-mercaptopurine riboside, mercaptopurine, 5-fluorouracil alone or in combination with leucovorin, tegafur, doxifluri-dine, carmofur, cytarabine, cytarabine ocfosfate, enocitabine, gemcitabine, fludarabin, 5-azacitidine, capecitabine, cladribine. clofarabine, decitabine, eflomithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, and vinorelbine;
Hormonal therapy agents include, but are not limited to, exemestane, Lupron, anastrozole, doxercalciferol, fadrozole, formestane, 11-beta hydroxysteroid dehydrogenase 1 inhibitors, 17-alpha hydroxylase/17,20 lyase inhibitors such as abiraterone acetate, 5-alpha reductase inhibitors such as finasteride and epristeride, anti-estrogens such as tamoxifen citrate and fulvestrant, Trelstar, toremifene, raloxifene, lasofoxifene, letrozole, anti-androgens such as bicalutamide, flutamide, mifepristone, nilutamide, Casodex, and anti-progesterones and combinations thereof;
Plant-derived anti-tumor substances include, e.g., those selected from mitotic inhibitors, for example epothilones such as sagopilone, ixabepilone and epothilone B, vinblastine, vinfiunine, docetaxel, and paclitaxel;
Cytotoxic topoisomerase inhibiting agents include, but are not limited to, aclarubicin, doxorubicin, amonafide, belotecan, camptothecin, 10-hydroxycamptothecin, 9-aminocampto-thecin, difiomotecan, irinotecan, topotecan, edotecarin, epimbicin, etoposide, exatecan, gimatecan, lurtotecan, mitoxantrone, pirambicin, pixantrone, rubitecan, sobuzoxane, tafluposide, and combinations thereof;
Immunologicals include interferons such as interferon alpha, interferon alpha-2a, interferon alpha-2b, interferon beta, interferon gamma-la and interferon gamma-nl, and other immune enhancing agents such as L19-IL2 and other IL2 derivatives, filgrastim, lentinan, sizofilan, TheraCys, ubenimex, aldesleukin, alemtuzumab, BAM-002, dacarbazine, daclizumab, deni-leukin, gemtuzumab, ozogamicin, ibritumomab, imiquimod, lenograstim, lentinan, melanoma vaccine (Corixa), molgramostim, sargramostim, tasonermin, tecleukin, thymalasin, tositumomab, Vimlizin, epratuzumab, mitumomab, oregovomab, pemtumomab, and Provenge; Merial melanoma vaccine;
Biological response modifiers are agents that modify defense mechanisms of living organisms or biological responses such as survival, growth or differentiation of tissue cells to direct them to have anti-tumor activity; such agents include, e.g., krestin, lentinan, sizofiran, picibanil, ProMune, and ubenimex;
Anti-angiogenic compounds include, but are not limited to, acitretin, aflibercept, angiostatin, aplidine, asentar, axitinib, recentin, bevacizumab, brivanib alaninat, cilengtide, combretastatin, DAST, endostatin, fenretinide, halofuginone, pazopanib, ranibizumab, rebimastat, removab, revlimid, sorafe ib, vatalanib, squalamine, sunitinib, telatinib, thalidomide, ukrain, and vitaxin;
Antibodies include, but are not limited to, trastuzumab, cetuximab, bevacizumab, rituximab, ticilimumab, ipilimumab, lumiliximab, catumaxomab, atacicept, oregovomab, and alemtuzumab;
VEGF inhibitors such as, e.g., sorafenib, DAST. bevacizumab. sunitinib, recentin, axitinib, aflibercept, telatinib, brivanib alaninate, vatalanib, pazopanib, and rambizumab; Palladia·EGFR (HER1) inhibitors such as, e.g., cetuximab, panitumumab, vectibix, gefitmib, erlotinib, and Zactima;
HER2 inhibitors such as, e.g., lapatinib, tratuzumab, and pertuzumab;
mTOPv inhibitors such as, e.g., temsirolimus, sirolimus/Rapamycin, and everolimus;
c-Met inhibitors;
PI3 and A T inhibitors;
CDK inhibitors;
Spindle assembly checkpoints inhibitors and targeted anti-mitotic agents such as PL inhibitors, Aurora inhibitors (e.g. Hesperadin), checkpoint kinase inhibitors, and KSP inhibitors;
HDAC inhibitors such as, e.g., panobinostat, vorinostat, MS275, belinostat, and LBH589;
HSP90 and HSP70 inhibitors;
Proteasome inhibitors such as bortezomib and carfilzomib;
Serine/threonine kinase inhibitors including ME inhibitors (such as e.g. RDEA 1 19) and Raf inhibitors such as sorafenib;
Farnesyl transferase inhibitors such as, e.g., tipifarnib;
Tyrosine kinase inhibitors including, e.g., dasatinib, nilotibib, DAST, bosutinib, sorafenib, bevacizumab, sunitinib, AZD2171, axitinib, aflibercept, telatinib, imatinib mesylate, brivanib alaninate, pazopanib, rambizumab, vatalanib, cetuximab, panitumumab, vectibix, gefitinib, erlotinib, lapatinib, tratuzumab, pertuzumab, and c-Kit inhibitors; Palladia, masitinib
Vitamin D receptor agonists;
Bcl-2 protein inhibitors such as obatoclax, oblimersen sodium, and gossypol;
Cluster of differentiation 20 receptor antagonists such as, e.g., rituximab;
Ribonucleotide reductase inhibitors such as, e.g., gemcitabine;
Tumor necrosis apoptosis inducing ligand receptor 1 agonists such as, e.g., mapatumumab;
5-Hydroxytryptamine receptor antagonists such as, e.g., rEV598, xaliprode, palonosetron hydrochloride, granisetron, Zindol, and AB-1001;
Integrin inhibitors including alpha5-betal integrin inhibitors such as, e.g., E7820, JSM 6425. volociximab, and endostatin;
Androgen receptor antagonists including, e.g., nandrolone decanoate, fluoxymesterone, Android, Prost-aid, andromustine, bicalutamide, flutamide, apo-cyproterone, apo-flutamide, chlormadinone acetate, Androcur, Tabi, cyproterone acetate, and nilutamide;
Aromatase inhibitors such as, e.g., anastrozole, letrozole, testolactone, exemestane, aminoglutethimide, and formestane;
Matrix metalloproteinase inhibitors;
Other anti-cancer agents including, e.g., alitretinoin, ampligen, atrasentan bexarotene, bortezomib, bosentan, calcitriol, exisulind, fotemustine, ibandronic acid, miltefosine, mitoxantrone, I-asparaginase, procarbazine, dacarbazine, hydroxycarbamide, pegasparga.se, pentostatin, tazaroten, velcade, gallium nitrate, canfosfamide, darinaparsin, and tretinoin.

Of course, other chemotherapeutic agents which are known to those of ordinary skill in the art can readily be substituted as this list should not be considered exhaustive or limiting.

The NSD3 and/or BET inhibitors may also be employed in cancer treatment in conjunction with radiation therapy and/or surgical intervention. Furthermore, the NSD3 and/or BET inhibitors may be utilized, as such or in compositions, in research and diagnostics, or as analytical reference standards, and the like, which are well known in the art. Thus, another aspect of the present invention relates to drug combinations comprising at least one inventive NSD3 and/or BET inhibitor and/or pharmaceutically acceptable salts thereof together with at least one anti-retroviral drug, especially at least one of the drugs mentioned above.

In some embodiments, the pharmaceutical composition comprising a NSD3i and/or BETi as disclosed herein or derivatives thereof as disclosed herein can supplement the treatment of any known additional therapy, including, but not limited to, antibody administration, vaccine administration, administration of cytotoxic agents, natural amino acid polypeptides, nucleic acids, nucleotide analogues, and biologic response modifiers. In some embodiments, additional therapy is, for example, surgery, chemotherapy, radiotherapy, thermotherapy, immunotherapy, hormone therapy and laser therapy. In some embodiments, the additional therapy is chemotherapy. Two or more combined compounds may be used together or sequentially with the pharmaceutical composition comprising a NSD3i and/or BETi as disclosed herein or a derivative thereof. In some embodiments, a NSD3i and/or BETi as disclosed herein or derivatives thereof can be administered before the additional therapy, after the additional therapy or at the same time as the additional therapy. In some embodiments, a NSD3i and/or BETi as disclosed herein or a functional derivative thereof are administered a plurality of times, and in other embodiments, the additional therapies are also administered a plurality of times.

In some embodiments, a NSD3i and/or BETi as disclosed herein or a functional derivative thereof can also be administered in therapeutically effective amounts as a portion of an anti-cancer cocktail. An anti-cancer cocktail is a mixture, for example at least one NSD3i and/or BETi as disclosed herein or functional derivatives thereof is combined with one or more additional anti-cancer agents in addition to a pharmaceutically acceptable carrier for delivery. The use of anti-cancer cocktails as a cancer treatment is routine. Anti-cancer agents that are well known in the art and can be used as a treatment in combination with a NSD3i and/or BETi as disclosed herein or functional derivatives thereof as disclosed herein include, but are not limited to: Actinomycin D, Aminoglutethimide, Asparaginase, Bleomycin, Busulfan, Carboplatin, Carmustine, Chlorambucil, Cisplatin (cis-DDP), Cyclophosphamide, Cytarabine HCl (Cytosine arabinoside), Dacarbazine, Dactinomycin, Daunorubicin HCl, Doxorubicin HCl, Estramustine phosphate sodium, Etoposide (V16-213), Flosuridine, S-Fluorouracil (5-Fu), Flutamide, Hydroxyurea (hydroxycarb amide), Ifosfamide, Interferon Alpha-2 a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Ockeotide, Paclitaxel; Plicamycin, Procarbazine HCl, Streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate, and analogues thereof. In some embodiments, the anti-cancer agent is selected from the group consisting of paclitaxel, cisplatin, doxorubicin and paclitaxel, vermurafib.

In certain embodiments, the pharmaceutical compositions comprising a NSD3i and/or BETi as disclosed herein or functional derivatives thereof can optionally further comprise one or more additional therapies or agents. In certain embodiments, the additional agent or agents are anti-cancer agents. In some embodiments, the therapeutic agents are chemotherapeutic agents, for example cisplatin, paxicital etc. In some embodiments, the therapeutic agents are radiotherapeutic agents. Examples of chemotherapeutic agents in the pharmaceutical compositions of this invention are, for example nitrogen mustards such as cyclophosphamide, ifosfamide, and melphalan; ethylenimines and methylmelamines such as hexamethylmelamine and thiotepa; pyrimidine analogs such as fluorouracil and fluorodeoxyuridine; vinca alkaloids such as vinblastine; epipodophyllotoxins such as etoposide and teniposide; antibiotics such as actinomycin D, doxorubicin, bleomycin, and mithramycin; biological response modifiers such as interferon, platinum coordination complexes such as cisplatin and carboplatin; estrogens such as diethylstilbestrol and ethinyl estradiol; antiandrogens such as flutamine; and gonadotropin releasing hormone analogs such as leuprolide. Other compounds such as decarbazine, nitrosoureas, methotrexate, diticene, and procarbazine are also effective. Of course, other chemotherapeutic agents which are known to those of ordinary skill in the art can readily be substituted as this list should not be considered exhaustive or limiting.

In some embodiments, a NSD3i and/or BETi as disclosed herein or functional derivatives thereof is administered to a subject with other anti-cancer therapies, for example cancer therapies to which the cancer was previously resistant or refractory.

In some embodiments, the methods of the present invention are directed to use of a NSD3i and/or BETi as disclosed herein and functional derivatives thereof with other therapeutic agents, for example chemotherapy agents as disclosed herein can be used at a lower dose that results in decreased side effects.

In certain embodiments, the endogenous compounds are isolated and/or purified or substantially purified by one or more purification methods described herein or known by those skilled in the art. Generally, the purities are at least 90%, in particular 95% and often greater than 99%. In certain embodiments, the naturally occurring compound is excluded from the general description of the broader genus.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can perform its intended function. The term “pharmaceutically acceptable carriers” is intended to include all solvents, diluents, or other liquid vehicle, dispersion or suspension aids, surface active agents, isotonic agents, thickening or emulsifying agents, preservatives, solid binders, lubricants and the like, as suited to the particular dosage form desired. Typically, such compounds are carried or transported from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its functional derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention.

These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use. These compounds include the zwitterionic forms, where possible, of compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “prodrug” refers to compounds that are rapidly transformed in vivo to yield the compounds of the invention, for example the pyrazoloathrone and functional derivatives thereof of the invention, by hydrolysis in blood. A thorough discussion is provided in T. Higachi and V. Stella, “Prodrugs as Novel Delivery Systems,” Vol. 14 of the A.C.S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of a compound, to mask side effects or toxicity, to improve the flavor of a compound or to alter other characteristics or properties of a compound. By virtue of knowledge of pharmacodynamic processes and drug metabolism in vivo, once a pharmaceutically active compound is identified, those of skill in the pharmaceutical art generally can design prodrugs of the compound (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

In other embodiments of the present invention, a NSD3i and/or BETi as disclosed herein or a functional derivative thereof are conjugated or covalently attached to another targeting agent to increase the specificity of a NSD3i and/or BETi and functional derivatives thereof targeting the cell, for example a cancer cell. Targeting agents can include, for example without limitation, antibodies, cytokines and receptor ligands. In some embodiments, the targeting agent is overexpressed on the cells to be targeted, for example the cancer cells as compared to normal cells. In alternative embodiments, the NSD3i and/or BETi and functional derivatives thereof can be conjugated or covalently attached to compounds that elicit an immune response, such as for example but without limitation, cytokines.

In some embodiments, a NSD3i and/or BETi as disclosed herein or a functional derivative thereof can be conjugated to, by covalent linkage or any other means, to another agent, for example a chemotherapy agent or antibody targeting a cancer cell or cancer stem cell. In some embodiments, a NSD3i and/or BETi as disclosed herein or a functional derivative thereof can be conjugated to a targeting moiety, for example a cancer cell targeting moiety to target the compounds of the present invention to a cancer cell. Such targeting moieties and methods are well known by persons of ordinary skill in the art and are encompassed for use in the methods of the present invention. The conjugation may be a permanent or reversible conjugation.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. In one aspect, a solution of resolvin and/or protectin or precursor or analog thereof can be administered as eye drops for ocular neovascularization or ear drops to treat otitis.

Liquid dosage forms for oral administration of the compounds of the invention include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some instances, pharmaceutical compositions comprising the resolvins and protectins of the invention for the administration of angiogenesis may be in a formulation suitable for rectal or vaginal administration, for example as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the compounds (resolvins and/or protectins and/or precursors or analogues thereof) of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

More specifically, the invention features an article of manufacture that contains packaging material and at least one compound of the present invention, for example a NSD3i and/or BETi as disclosed herein or a functional derivative thereof are contained within the packaging material. The packaging material contains a label or package insert indicating that the formulation can be administered to the subject with neovascularization in an amount, at a frequency, and for a duration effective treat or prevent symptoms associated with such disease states or conditions discussed throughout this specification. In some embodiments, the proliferative disorder is a cancer.

Remington's Pharmaceutical sciences Ed. Germany, Merk Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its functional derivatives such as sodium carboxymethyl cellulose, ethyl cellulose and cellulose acetate; malt; gelatin; talc; excipients such as cocoa butter and: suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; water; isotonic saline; Ringer's solution, ethyl alcohol, and phosphate buffer solutions, as well as other non-toxic compatible lubricants such as sodium lauryl sulfate and magnesium sulfate, as well as coloring agents, releasing agents, coating agents, sweetening, flavoring and perfuming agents, preservatives and antioxidants can also be present in the composition, according to the judgment of the formulator.

Oligonucleotide Formulations:

A formulated oligonucleotide composition can assume a variety of states. In some examples, the composition can be at least partially crystalline, uniformly crystalline, and/or anhydrous (e.g., less than 80, 50, 30, 20, or 10% water). In another example, the oligonucleotide is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a micro particle as can be appropriate for a crystalline composition). Generally, the oligonucleotide composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated in combination with another agent, e.g., another therapeutic agent or an agent that stabilizes the oligonucleotide, e.g., a protein that complex with oligonucleotide to form an oligonucleotide-protein compelx Still other agents include chelators, e.g., EDTA (e.g., to remove divalent cations such as Mg2+), salts, DNAse inhibitors, RNAse inhibitors (e.g., a broad specificity RNAse inhibitor such as RNAsin) and so forth.

In some embodiments, the oligonucleotide preparation includes at least a second therapeutic agent (e.g., an agent other than RNA or DNA). Exemplary therapeutic agents that can formulated with an oligonucleotide preparation include, but are not limited to, those found in Harrison's Principles of Internal Medicine, 17th Edition, 2008, McGraw-Hill N.Y., NY; Physicians Desk Reference, 63rd Edition, 2008, Thomson Reuters, N.Y., N.Y.; Goodman & Gilman's The Pharmacological Basis of Therapeutics, 11th Edition, 2005, McGraw-Hill N.Y., NY; United States Pharmacopeia, The National Formulary, USP-32 NF-27, 2008, U.S. Pharmacopeia, Rockville, Md., the complete contents of all of which are incorporated herein by reference.

In some embodiments, the second therapeutic agent is an anti-hypertension agent or anti-hypertensive.

Exemplary Oligonucleotide Formulations

Liposomes:

The oligonucleotides of the invention can be formulated in liposomes. As used herein, a liposome is a structure having lipid-containing membranes enclosing an aqueous interior. Liposomes can have one or more lipid membranes. In some embodiments, liposomes have an average diameter of less than about 100 nm. More preferred embodiments provide liposomes having an average diameter from about 30-70 nm and most preferably about 40-60 nm. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand.

Liposome compositions can be prepared by a variety of methods that are known in the art. See e.g., U.S. Pat. Nos. 4,235,871; 4,737,323; 4,897,355 and 5,171,678; published International Applications WO 96/14057 and WO 96/37194; Feigner, P. L. et al., Proc. Natl. Acad. Sci., USA (1987) 8:7413-7417, Bangham, et al. M. Mol. Biol. (1965) 23:238, Olson, et al. Biochim Biophys. Acta (1979) 557:9, Szoka, et al. Proc. Natl. Acad. Sci. (1978) 75: 4194, Mayhew, et al. Biochim Biophys. Acta (1984) 775:169, Kim, et al. Biochim Biophys. Acta (1983) 728:339, and Fukunaga, et al. Endocrinol. (1984) 115:757.

Micelles and Other Membranous Formulations:

The oligonucleotides of the invention can be prepared and formulated as micelles. As used herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In some embodiments, the formulations comprises micelles formed from an oligonucleotide of the invention and at least one amphiphilic carrier, in which the micelles have an average diameter of less than about 100 nm, preferably. More preferred embodiments provide micelles having an average diameter less than about 50 nm, and even more preferred embodiments provide micelles having an average diameter less than about 30 nm, or even less than about 20 nm.

Micelle formulations can be prepared by mixing an aqueous solution of the oligonucleotide composition, an alkali metal C8 to C22 alkyl sulphate, and an amphiphilic carrier. The amphiphilic carrier can be added at the same time or after addition of the alkali metal alkyl sulphate. Micelles will form with substantially any kind of mixing of the ingredients but vigorous mixing in order to provide smaller size micelles.

Emulsions:

The oligonucleotides of the present invention can be prepared and formulated as emulsions. As used herein, “emulsion” is a heterogenous system of one liquid dispersed in another in the form of droplets. Emulsions are often biphasic systems comprising two immiscible liquid phases intimately mixed and dispersed with each other. Either of the phases of the emulsion can be a semisolid or a solid, as is the case of emulsion-style ointment bases and creams. The oligonucleotide can be present as a solution in either the aqueous phase, oily phase or itself as a separate phase.

In some embodiments, the compositions are formulated as microemulsions. As used herein, “microemulsion” refers to a system of water, oil and amphiphile which is a single optically isotropic and thermodynamically stable liquid solution. Microemuslions also include thermodynamically stable, isotropically clear dispersions of two immiscible liquids that are stabilized by interfacial films of surface-active molecules.

The application of emulsion formulations via dermatological, oral and parenteral routes and methods for their manufacture have been reviewed in the literature, for example see Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., volume 1, p. 245; and Block, in Pharmaceutical Dosage Forms, Lieberman, Rieger and Banker (Eds.), 1988, Marcel Dekker, Inc., New York, N.Y., volume 1, p. 335, contents of which are herein incorporated by reference in their entirety.

Lipid Particles:

The oligonucleotides of the present invention can be prepared and formulated as lipid particles, e.g., formulated lipid particles (FLiPs) comprising (a) an oligonucleotide of the invention, where said oligonucleotide has been conjugated to a lipophile and (b) at least one lipid component, for example an emulsion, liposome, isolated lipoprotein, reconstituted lipoprotein or phospholipid, to which the conjugated oligonucleotide has been aggregated, admixed or associated. The stoichiometry of oligonucleotide to the lipid component can be 1:1. Alternatively the stoichiometry can be 1:many, many:1 or many:many, where many is two or more.

The FLiP can comprise triacylglycerols, phospholipids, glycerol and one or several lipid-binding proteins aggregated, admixed or associated via a lipophilic linker molecule with an oligonucleotide. Surprisingly, it has been found that due to said one or several lipid-binding proteins in combination with the above mentioned lipids, the FLiPs show affinity to liver, gut, kidney, steroidogenic organs, heart, lung and/or muscle tissue. These FLiPs can therefore serve as carrier for oligonucleotides to these tissues. For example, lipid-conjugated oligonucleotides, e.g., cholesterol-conjugated oligonucleotides, bind to HDL and LDL lipoprotein particles which mediate cellular uptake upon binding to their respective receptors thus directing oligonucleotide delivery into liver, gut, kidney and steroidogenic organs, see Wolfrum et al. Nature Biotech. (2007), 25:1145-1157.

The FLiP can be a lipid particle comprising 15-25% triacylglycerol, about 0.5-2% phospholipids and 1-3% glycerol, and one or several lipid-binding proteins. FLiPs can be a lipid particle having about 15-25% triacylglycerol, about 1 2% phospholipids, about 2-3% glycerol, and one or several lipid-binding proteins. In some embodiments, the lipid particle comprises about 20% triacylglycerol, about 1.2% phospholipids and about 2.25% glycerol, and one or several lipid-binding proteins.

Another suitable lipid component for FLiPs is lipoproteins, for example isolated lipoproteins or more preferably reconstituted lipoprotieins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins). Methods of producing reconstituted lipoproteins are known in the art, for example see A. Jones, Experimental Lung Res. 6, 255-270 (1984), U.S. Pat. Nos. 4,643,988 and 5,128,318, PCT publication WO87/02062, Canadian Pat. No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

One preferred lipid component for FLiP is Intralipid. Intralipid® is a brand name for the first safe fat emulsion for human use. Intralipid® 20% (a 20% intravenous fat emulsion) is made up of 20% soybean oil, 1.2% egg yolk phospholipids, 2.25% glycerin, and water for injection. It is further within the present invention that other suitable oils, such as saflower oil, can serve to produce the lipid component of the FLiP.

FLiP can range in size from about 20-50 nm or about 30-50 nm, e.g., about 35 nm or about 40 nm. In some embodiments, the FLiP has a particle size of at least about 100 nm FLiPs can alternatively be between about 100-150 nm, e.g., about 110 nm, about 120 nm, about 130 nm, or about 140 nm, whether characterized as liposome- or emulsion-based. Multiple FLiPs can also be aggregated and delivered together, therefore the size can be larger than 100 nm.

The process for making the lipid particles comprises the steps of: (a) mixing a lipid components with one or several lipophile (e.g. cholesterol) conjugated oligonucleotides that can be chemically modified; and (b) fractionating this mixture. In some embodiments, the process comprises the additional step of selecting the fraction with particle size of 30-50 nm, preferably of about 40 nm in size.

Some exemplary lipid particle formulations amenable to the invention are described in U.S. patent application Ser. No. 12/412,206, filed Mar. 26, 2009, contents of which are herein incorporated by reference in their entirety.

Yeast Cell Wall Particles:

In some embodiments, the oligonucleotide is formulated in yeast cell wall particles (“YCWP”). A yeast cell wall particle comprises an extracted yeast cell wall exterior and a core, the core comprising a payload (e.g., oligonucleotides). Exterior of the particle comprises yeast glucans (e.g. beta glucans, beta-1,3-glucans, beta-1,6-glucans), yeast mannans, or combinations thereof. Yeast cell wall particles are typically spherical particles about 1-4 μm in diameter.

Preparation of yeast cell wall particles is known in the art, and is described, for example in U.S. Pat. Nos. 4,992,540; 5,082,936; 5,028,703; 5,032,401; 5,322,841; 5,401,727; 5,504,079; 5,607,677; 5,741,495; 5,830,463; 5,968,811; 6,444,448; and 6,476,003, U.S. Pat. App. Pub. Nos. 2003/0216346 and 2004/0014715, and Int. App. Pub. No. WO 2002/12348, contents of which are herein incorporated by reference in their entirety. Applications of yeast cell like particles for drug delivery are described, for example in U.S. Pat. Nos. 5,032,401; 5,607,677; 5,741,495; and 5,830,463, and U.S. Pat. Pub Nos. 2005/0281781 and 2008/0044438, contents of which are herein incorporated by reference in their entirety. U.S. Pat. App. Pub. No. 2009/0226528, contents of which are herein incorporated by reference, describes formulation of nucleic acids with yeast cell wall particles for delivery of oligonucleotide to cells.

Additional exemplary formulations for oligonucleotides are described in U.S. Pat. Nos. 4,897,355; 4,394,448; 4,235,871; 4,231,877; 4,224,179; 4,753,788; 4,673,567; 4,247,411; 4,814,270; 5,567,434; 5,552,157; 5,565,213; 5,738,868; 5,795,587; 5,922,859; and 6,077,663, Int. App. Nos. PCT/US07/079203, filed Sep. 21, 2007; PCT/US07/080331, filed Oct. 3, 2007; U.S. patent application Ser. No. 12/123,922, filed May 28, 2008; U.S. Pat. Pub. Nos. 2006/0240093 and 2007/0135372 and U.S. Provisional App. No. 61/018,616, filed Jan. 2, 2008; 61/039,748, filed Mar. 26; 2008; 61/045,228, filed Apr. 15, 2008; 61/047,087, filed Apr. 22, 2008; 61/051,528, filed May 21, 2008; and 61/113,179 (filed Nov. 10, 2008), contents of which are herein incorporated by reference in their entirety. Behr (1994) Bioconjugate Chem. 5:382-389, and Lewis et al. (1996) PNAS 93:3176-3181), also describe formulations for oligonucleotides that are amenable to the invention, contents of which are herein incorporated by reference in their entirety.

Vectors: Vectors can be used to deliver NSD3i that are RNAi or oligonucleotides, e.g., a nucleic acid sequence encoding a decoy protein of SEQ ID NO: 6 or a fragment thereof. Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; marine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA.

Retroviruses have been approved for human gene therapy trials. Genetically altered retroviral expression vectors have general utility for the high efficiency transduction of nucleic acids in viva. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., New York (1990) and Murry, E. J. Ed. “Methods in Molecular L Biology,” vol. 7, Humana Press, Inc., Cliffton, N.J. (1991).

In some embodiments the “in vivo expression elements” are any regulatory nucleotide sequence, such as a promoter sequence or promoter-enhancer combination, which facilitates the efficient expression of the nucleic acid to produce the RNAi or decoy NSD3i. The in vivo expression element may, for example, be a mammalian or viral promoter, such as a constitutive or inducible promoter and/or a tissue specific promoter. Examples of which are well known to one of ordinary skill in the art. Constitutive mammalian promoters include, but are not limited to, polymerase promoters as well as the promoters for the following genes: hypoxanthine phosphoribosyl transferase (HPTR), adenine deaminase, pyruvate kinase, and beta.-actin. Exemplary viral promoters which function constitutively in eukaryotic cells include, but are not limited to, promoters from the simian virus, papilloma virus, adenovirus, human immunodeficiency virus (HIV), Rous sarcoma virus, cytomegalovirus, the long terminal repeats (LTR) of moloney leukemia virus and other retroviruses, and the thymidine kinase promoter of herpes simplex virus. Other constitutive promoters are known to those of ordinary skill in the art. Inducible promoters are expressed in the presence of an inducing agent and include, but are not limited to, metal-inducible promoters and steroid-regulated promoters. For example, the metallothionein promoter is induced to promote transcription in the presence of certain metal ions. Other inducible promoters are known to those of ordinary skill in the art.

Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, beta-galactosidase alpha-2,6 sialylkansferase promoter, insulin-like growth factor binding protein (IGFBP-1) promoter, aldolase B promoter, human transferrin promoter, and collagen type I promoter. Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific expression elements for gastric tissue include but are not limited to the human H+/K+-ATPase alpha subunit promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for adrenal cells include, but are not limited to, cholesterol side-chain cleavage (SCC) promoter. Exemplary tissue-specific expression elements for the general nervous system include, but are not limited to, gamma-gamma enolase (neuron-specific enolase, NSE) promoter. Exemplary tissue-specific expression elements for the brain include, but are not limited to, the neurofilament heavy chain (NF—H) promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′ transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter.

In some embodiments, tissue-specific expression elements for breast cells include, but are not limited to, the human alpha-lactalbumin promoter. Exemplary tissue-specific expression elements for the lung include, but are not limited to, the cystic fibrosis transmembrane conductance regulator (CFTR) gene promoter.

Other elements aiding specificity of expression in a tissue of interest can include secretion leader sequences, enhancers, nuclear localization signals, endosmolytic peptides, etc. Preferably, these elements are derived from the tissue of interest to aid specificity. In general, the in vivo expression element shall include, as necessary, 5′ non-transcribing and 5′ non-translating sequences involved with the initiation of transcription. They optionally include enhancer sequences or upstream activator sequences.

An NSD3i agent, e.g., a NSD3i RNAi agent or NSD3i decoy protein either alone, or expressed as a viral vector or complexed to targeting moieties can be delivered using any delivery system such as topical administration, subcutaneous, intramuscular, intraperitoneal, intrathecal and intravenous injections, catheters.

### Uses

In another embodiment, the present invention provides a method for treating a variety of conditions by administering an effective amount of example a NSD3i and/or BETi as disclosed herein or a functional derivative thereof to a subject in need thereof. Conditions that may be treated by the compounds of this invention, or a pharmaceutical composition containing the same, include any condition which is treated or results in the reduction of a symptom by administration of an inhibitor at least one member of the NSD3i and/or BETi, and thereby benefit from administration of a NSD3i and/or BETi as disclosed herein or a functional derivative thereof. Representative conditions in this regard include, for example, but not limited to, NMC or a NSD3-dependent cancer, including NSD3/NUT, BRD4/NUT and BRD3/NUT cancers, or a BRD-dependent, NUT-independent cancer. Such BRD-dependent, NUT-independent cancers include cancers that are responsive to BET inhibitors, and include, but are not limited to: leukemia, lymphoma, multiple myeloma, neuroblastoma, acute myeloid leukemia (AML), Burkitt lymphomia, Erythroleukemia, Lung adenocarcinoma, B-ALL (B-cell acute lymphoblastic leukemia), Burkitt Lymphoma, APML (Promyelocytic leukemia), Multiple myeloma, Cervical squamous cell carcinoma, Breast carcinoma, Prostate carcinoma or melanoma.

Accordingly, the present invention relates to the use of a NSD3i as disclosed herein or a functional derivative thereof for the treatment of any disorder where administration of an inhibitor of a BET inhibitor is whole, or part, of the therapeutic regime.

### Screening or Validating Potential NSD3 Inhibitor

The following refers to screening or validating potential NSD3 inhibitors, especially selective NSD3 inhibitors to be used in accordance with the present invention.

In some embodiments the present invention relates to a high throughput screen (HTS) to identify compounds which disrupt the interaction between NSD3 and a BET protein, e.g., BRD4 or BRD3. In some embodiments, an exemplary assay can be performed by one of ordinary skill in the art, for example, such an assay can comprise at least the N-terminal region of NSD3 which binds to the BET protein, and at least a portion of the BET protein which interacts with the NSD3 protein, and an agent which produces a florescent signal when NSD3 complexes with either BRD3 or BRD4. A NSD3 inhibitor which disrupts the interaction between NSD3 and a BET protein, e.g., BRD4 or BRD3 can be identified by identifying a decrease in the fluorescent signal on addition of the agent to the NSD3-BRD3/4 complex.

In some embodiments, the assay comprises at least a fragment of the NSD3 of SEQ ID NO: 4 (corresponding to the amino acid sequence of NSD3 which binds to the ET domain on a BET protein) and comprises at least a fragment of the ET domain of SEQ ID NO:6 (corresponding to the amino acid sequence of the ET domain of BRD4). In some embodiments, the fragment is at least 10 amino acids, or at least 20, or at least 30 or at least 40 or at least 50 or more amino acids of either SEQ ID NO: 4 or SEQ ID NO: 6. In some embodiments, the protein of NSD3 of SEQ ID NO: 4 (which interacts with the ET domain of BRD4) is expressed in a cell from a vector comprising the nucleic acid sequence SEQ ID NO: 3. In some embodiments, the protein of the ET domain of BRD4 corresponding to SEEEDKCKPMSYEEKRQLSLDINKLPGEKLGRVVHIIQSREPSLKNSNPDEIEIDFETLK PSTLRELERYVTSCLRKKRKPQ (SEQ ID NO: 6) (which interacts with NSD3) is expressed in a cell from a vector comprising the nucleic acid sequence SEQ ID NO: 5 (ET domain of BRD4), which is a follows:

TCGGAGGAAGAGGACAAGTGCAAGCCTATGTCCTATGAGGAGAAGCGG
CAGCTCAGCTTGGACATCAACAAGCTCCCCGGCGAGAAGCTGGGCCGC
GTGGTGCACATCATCCAGTCACGGGAGCCCTCCCTGAAGAATTCCAAC
CCCGACGAGATTGAAATCGACTTTGAGACCCTGAAGCCGTCCACACTG
CGTGAGCTGGAGCGCTATGTCACCTCCTGTTTGCGGAAGAAAAGGAAA
CCTCAAGCT.

In some embodiments, there is a florescent marker or other signal tag on either the protein of SEQ ID NO: 4 and/or SEQ ID NO: 6, or a portion thereof which produces a measurable signal when the proteins of SEQ ID NO:4 or SEQ ID NO: 6, or fragments thereof, interact and form a NSD3-ET domain complex. Accordingly, an agent which, when added to the NSD3-ET domain complex (of SEQ ID NO:4-SEQ ID NO:6, or fragments thereof) causes a decrease in the measurable signal (e.g., a decrease in the fluorescent signal) indicates a disruption in the interaction between a portion of NSD3 of SEQ ID NO: 4 and a portion of the ET domain of SEQ ID NO: 6, and is a potential NSD3 inhibitor for use in the methods and compositions as disclosed herein. It is preferred herein that a potential NSD3 inhibitor is identified by a decrease in measurable signal by at least 10%, 20%, 30%, 40%, 50%, 60%, 70%, 80% and most preferably by at least 90% in cell(s) contacted with/exposed to the agent, as compared with the measurable signal in (a) control cell(s), (a) control tissue(s) or (a) control cell culture(s).

In another example, screening or validating potential NSD3 inhibitors can measure the activity/level of expression of NSD3, wherein a lower activity/level of expression of NSD3 is compared to a control is indicative for the capacity of a candidate molecule/substance to selectively inhibit NSD3. The term “activity of NSD3” used herein refers to the activity of a NSD3 protein (protein encoded by a NSD3 gene). The term “expression of NSD3” is used herein interchangeably with “expression of NSD3 gene” and refers to the expression of the NSD3 gene. It is to be understood that the activity/expression level of NSD3 determined in (a) cell(s), (a) tissue(s) or (a) cell culture(s) contacted with/exposed to an NSD3 inhibitor is compared with the activity/expression level of NSD3 in (a) control cell(s), (a) tissue(s) or (a) cell culture(s), i.e. cell(s), (a) tissue(s) or (a) cell culture(s) not contacted with/exposed to an NSD3inhibitor. A skilled person will be aware of means and methods for performing such tests and selecting appropriate controls. Preferably, the control cell(s), (a) tissue(s) or (a) cell culture(s) will be identical to the cell(s), (a) tissue(s) or (a) cell culture(s) to be tested as described herein with the only exception that the control (s), (a) tissue(s) or (a) cell culture(s) are not contacted with/exposed to the NSD3 inhibitor.

Preferably, decreased NSD3 activity/expression levels of NSD3 proteins/polypeptides and/or NSD3 polynucleotides/nucleic acid molecules are indicative of the capacity of a candidate molecule/substance to selectively inhibit NSD3. It is preferred herein that the NSD3activity/expression level is decreased by at least 10%, 20%, 30%, 40%, 50%, 60%, 70%, 80% and most preferably by at least 90% in cell(s), (a) tissue(s) or (a) cell culture(s) contacted with/exposed to an NSD3 inhibitor compared with the activity/expression level of NSD3 in (a) control cell(s), (a) control tissue(s) or (a) control cell culture(s). It is of note that the NSD3 activity must not necessarily correlate with the expression level. Thus, it may be that NSD3 activity is decreased in the presence of an NSD3 inhibitor even though NSD3 expression is the same or even increased. However, a person skilled of the art will be aware of this and preferably evaluate NSD3 activity (i.e. activity/function of the NSD3 protein) when determining the capacity of a candidate substance to inhibit NSD3.

As mentioned, a person skilled in the art will be aware of corresponding means and methods for detecting and evaluating the NSD3 activity/expression level. Exemplary methods to be used include but are not limited to molecular assessments such as Western Blots, Northern Blots, Real-Time PCR and the like.

If the gene product is an RNA, in particular an mRNA (e.g. unspliced, partially spliced or spliced mRNA), determination can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR. These and other suitable methods for binding (specific) mRNA are well known in the art and are, for example, described in Sambrook and Russell (2001). A skilled person is capable of determining the amount of the component, in particular said gene products, by taking advantage of a correlation, preferably a linear correlation, between the intensity of a detection signal and the amount of the gene product to be determined.

In case the component is a polypeptide/protein, quantification can be performed by taking advantage of the techniques referred to above, in particular Western blotting techniques. Generally, the skilled person is aware of methods for the quantitation of (a) polypeptide(s)/protein(s). Amounts of purified polypeptide in solution can be determined by physical methods, e.g. photometry. Methods of quantifying a particular polypeptide in a mixture rely on specific binding, e.g., of antibodies. Specific detection and quantitation methods exploiting the specificity of antibodies comprise for example immunohistochemistry {in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction. Alternatively, protein quantitation methods may involve but are not limited to mass spectrometry or enzyme-linked immunosorbant assay methods.

Also the use of high throughput screening (HTS) is envisaged in context of the present invention, in particular the screening methods for potential NSD3 inhibitors to be used herein. Suitable (HTS) approaches are known in the art and a person skilled in the art is readily in the position to adapt such protocols or known HTS approaches to the performance of the methods of the present invention.

Screening-assays are usually performed in liquid phase, wherein for each cell/tissue/cell culture to be tested at least one reaction batch is made. Typical containers to be used are micro titer plates having for example, 384, 1536, or 3456 wells (i.e. multiples of the “original” 96 reaction vessels). Robotics, data processing and control software, and sensitive detectors, are further commonly used components of a HTS device. Often robot system are used to transport micro titer plates from station to station for addition and mixing of sample(s) and reagent(s), incubating the reagents and final readout (detection). Usually, HTS can be used in the simultaneous preparation, incubation and analysis of many plates.

The assay can be performed in a singly reaction (which is usually preferred), may, however, also comprise washing and or transfer steps. Detection can be performed taking advantage of radioactivity, luminescence or fluorescence, like fluorescence-resonance-energy transfer (FRET) and fluorescence polarisation (FP) and the like. The biological samples described herein can also be used in such a context. In particular cellular assays and in vivo assays can be employed in HTS. Cellular assays may also comprise cellular extracts, i.e. extracts from cells, tissues and the like. However, preferred herein is the use of cell(s) or tissue(s) as biological sample (in particular a sample obtained from a patient/subject suffering or being prone to suffer from midline carcinoma, especially NMC), whereas in vivo assays (wherein suitable animal models are employed, e.g. the herein described mouse models) are particularly useful in the validation of potential NSD3 inhibitors. Depending on the results of a first assay, follow up assays can be performed by re-running the experiment to collect further data on a narrowed set (e.g. samples found “positive” in the first assay), confirming and refining observations.

HTS is useful in identifying further NSD3 inhibitors to be used herein. The screening of compound libraries with usually several hundred thousands of substances takes usually between days and weeks. An experimental high throughput screen may be supplemented (or even be replaced) by a virtual screen. For example, if the structure of the target molecule (e.g. NSD3) is known, methods can be employed, which are known under the term “docking”. If the structure of several target-binding molecules is known (e.g. the herein described NSD3) methods for Pharmacophor-Modelling can be used aiming at the development new substances which also bind to the target molecule. A suitable readout in animal (in vivo) models is tumor growth (or respectively the complete or partial inhibition of tumor growth and/or its remission).

High-throughput methods for the detection of mutations involve massively parallel sequencing approaches, such as the “picotiter plate pyrosequencing”. This approach relies on emulsion PCR-based clonal amplification of a DNA library adapted onto micron-sized beads and subsequent pyrosequencing-by-synthesis (Thomas R et al. Nature Med 2007) of each clonally amplified template in a picotiter plate, generating over 200,000 unique clonal sequencing reads per experiment. Furthermore, mass spectrometric genotyping approaches (Thomas R K et al.; Nat Gen 2007) and other next generation sequencing methods (Marguerat S et al.; Biochem Soc Trans 2008) may be employed.

The meaning of the terms “cell(s)”, “tissue(s)” and “cell culture(s)” is well known in the art and may, for example, be deduced from “The Cell” (Garland Publishing, Inc., third edition). Generally, the term “cell(s) used herein refers to a single cell or a plurality of cells. The term “plurality of cells” means in the context of the present invention a group of cells comprising more than a single cell. Thereby, the cells out of said group of cells may have a similar function. Said cells may be connected cells and/or separate cells. The term “tissue” in the context of the present invention particularly means a group of cells that perform a similar function. The term “cell culture(s)” means in context of the present invention cells as defined herein above which are grown/cultured under controlled conditions. Cell culture(s) comprise in particular cells (derived/obtained) from multicellular eukaryotes, preferably animals as defined elsewhere herein. It is to be understood that the term “cell culture(s)” as used herein refers also “tissue culture (s)” and/or “organ culture(s)”, an “organ” being a group of tissues which perform the same function.

Preferably, the cell(s), tissue(s) or cell culture(s) to be contacted with/exposed to a selective NSD3 inhibitor comprise/are derived from or are (a) tumor cell(s). The tumor cells may, for example, be obtained from a biopsy, in particular a biopsy/biopsies from a patient/subject suffering from midline carcinoma, like NMC or, though less preferred a patient/subject being prone to suffer from midline carcinoma, like NMC. It is preferred herein that said subject is a human. The term “mammalian tumor cell(s)” used herein refers to (a) tumor cell(s) which is derived from or is a tumor cell from a mammal, the term mammal being derived herein below. As described herein above in respect of “cell(s)”, “tissue(s)” and “cell culture(s)” the “mammalian tumor cells” may be obtained from a biopsy, in particular a biopsy/biopsies from a patient/subject suffering from midline carcinoma, like NMC or, though less preferred a patient/subject being prone to suffer from midline carcinoma, like NMC. The term “tumor cell” also relates to “cancer cells”. Generally, said tumor cell or cancer cell may be obtained from any biological source/organism, particularly any biological source/organism, suffering from the above-mentioned midline carcinoma, like NMC.

Preferably, the (tumor) cell(s) or (cancer) cell to be contacted is (are) obtained/derived from an animal. More preferably, said (tumor)/(cancer) cell(s) is (are) derived from a mammal. The meaning of the terms “animal” or “mammal” is well known in the art and can, for example, be deduced from Wehner und Gehring (1995: Thieme Verlag). Non-limiting examples for mammals are even-toed ungulates such as sheep, cattle and pig, odd-toed angulates such as horses as well as cats and dogs. In the context of this invention, it is particularly envisaged that DNA samples are derived from organisms that are economically, agronomically or scientifically important. Scientifically or experimentally important organisms include, but are not limited to, mice, rats, rabbits, guinea pigs and pigs.

The tumor cell(s) may also be obtained from primates which comprise lemurs, monkeys and apes. The meaning of the terms “primate”, “lemur”, “monkey” and “ape” is known and may, for example, be deduced by an artisan from Wehner und Gehring (1995, Thieme Verlag). As mentioned above, the tumor or cancer cell(s) is (are) most preferably derived from a human being suffering from the above-mentioned NMCs. In context of this invention particular useful cells, in particular tumor or cancer cells, are, accordingly, human cells. These cells can be obtained from e.g. biopsies or from biological samples but the term “cell” also relates to in vitro cultured cells.

In some embodiments, a preferred, however non-limiting cell(s) or cell culture(s) also used in the appended example is cell line 1221 as disclosed herein (showing translocation resulting in the formation of a NSD3-NUT-fusion protein. Other cell lines with the BRD4/NUT or BRD3/NUT oncoproteins can also be used, such as for example, cell line 143100 (showing a tl 5; 19 translocation resulting in the formation of a BRD4-NUT-fusion protein) which are well known in the art.

The following explanations refer, inter alia, to rearrangements in the NUT gene which is characteristic especially for NUT midline carcinoma. The below explanations concerning the NUT gene and NUT protein, apply, mutatis mutandis, to other nucleic acid sequences and amino acid sequences to be employed in context of the present invention, such as partner genes of NUT in NUT fusion genes like BRD4-NUT fusion genes, BRD3-NUT fusion genes or NUT-variant fusion genes characteristic for NMC. Accordingly, the explanations apply, mutatis mutandis, to members of the BET family (BRD2, BRDT and. in particular, human BRD3 gene and BRD3 protein (SEQ ID NOs: 5 and 6, respectively) and human BRD4 gene and BRD4 protein (SEQ ID NOs: 3 and 4, respectively).

### Kits

In another embodiment, this invention provides kits for the practice of the methods of this invention. The kits preferably include one or more containers containing an NSD3 and/or BET inhibitor and a pharmaceutically acceptable excipient. The kit may optionally contain additional therapeutics to be co-administered with the NSD3i and/or BETi agent.

The kits may also optionally include appropriate systems (e.g. opaque containers) or stabilizers (e.g. antioxidants) to prevent degradation of the NSD3i and/or BETi by light or other adverse conditions.

The kits may optionally include instructional materials containing directions (i.e., protocols) providing for the use of the NSD3i and/or BETi in the treatment of cancer, such as NMC, or a subject with NSD3/NUT or BRD4/NUT or BRD3/NUT gene fusions. In particular the disease can include any one or more of the disorders described herein including, any NSD3-dependent cancer, or any BRD4-dependent cancer (which is independent of NUT).

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments, the present invention may be defined in any of the following numbered paragraphs:

1. A method for treating cancer in a subject comprising administering to the subject an effective amount of a pharmaceutical composition comprising a NSD3 inhibitor (NDSi).
2. The method of paragraph 1, wherein the cancer is a NSD3-dependent cancer.
3. The method of any of paragraphs 1 to 2, wherein the NSD3-depdendent cancer is NUT midline carcinoma (NMC) characterized by the presence of at least one rearrangement of the NUT gene in a NMC tumor or cancer cell.
4. The method of any of paragraphs 1 to 3, wherein the rearrangement in the NUT gene is a translocation of the NUT gene to form at least one of: a NSD3/NUT fusion gene, a BRD4/NUT fusion gene or a BRD3/NUT fusion gene.
5. The method of any of paragraphs 1 to 4, wherein the cancer is selected from the group consisting of: primary breast carcinoma, pancreatic adenocarcinoma, acute myeloid leukemia or myelodysplastic syndrome with NUP98-NSD3 fusion oncogene.
6. The method of any of paragraphs 1 to 5, wherein the cancer is selected from the group consisting of: leukemia, lymphoma, multiple myeloma, neuroblastoma, acute myeloid leukemia (AML), Burkitt lymphomia, Erythroleukemia, Lung adenocarcinoma, B-ALL (B-cell acute lymphoblastic leukemia), Burkitt Lymphoma, APML (Promyelocytic leukemia), Multiple myeloma, Cervical squamous cell carcinoma, Breast carcinoma, Prostate carcinoma or melanoma.
7. The method of any of paragraphs 1 to 6, wherein the NSD3 inhibitor is selected from the group consisting of antibodies, antibody fragments, RNAi, siRNA, a NSD3 decoy molecule, or a polypeptide that blocks the binding of NSD3 with BRD4 and/or BRD3.
8. The method of any of paragraphs 1 to 7, wherein the NSD3 decoy molecule comprises a portion of the ET domain of BRD4.
9. A method for treating NUT midline carcinoma (NMC) characterized by the rearrangement of the NUT gene to form a NSD3/NUT fusion gene, comprising administering to the subject an effective amount of a BET inhibitor (BETi).
10. The method of paragraph 9, wherein the BET inhibitor is a BRD4 inhibitor.
11. The method of paragraphs 9 or 10, wherein the BET inhibitor is selected from the group consisting of: JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), GSK-525762A, LY294002, 1-[2-(1/-/-benzimidazol-2-ylthio)ethyl]-1,3-dihydro-3-methyl-2H-benzinidazole-2-thione, 1-methylethyl ((2S,4R)-1-acetyl-2-methyl-6-{4-[(methylamino)methyl]phenyl}-1,2,3,4-tetrahydro-4-quinolinyl)carbamate, 2-[(4S)-6-(4-Chlorophenyl)-1-methyl-8-(methyloxy)-4H-[1,2,4]triazolo[4,3-a][1,4]benzodiazepin-4-yl]-N-ethylacetamide, 7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one, 7-(3,5-dimethyl-4-isoxazolyl)-8-(methoxy)-1-[(1R)-phenylethyl]-2-(tetrahydro-2H-pyran-4-yl)-1H-imidazo[4,5-c]quinolone, 4-{(2S,4R)-1-acetyl-4-[(4-chlorophenyl)amino]-2-methyl-1,2,3,4-tetrahydro-6-quinolinyl}benzoic acid, and N-{1-methyl-7-[4-(1-piperidinylmethyl)phenyl][1,2,4]triazolo [4,3-a]quinolin-4-yl}urea.
12. The method of paragraph 9, comprising an initial step of detecting the presence of NSD3/NUT fusion oncoprotein or NSD3/NUT fusion gene prior to administering an effective amount of a BET inhibitor.
13. Use of a NSD3 inhibitor for the manufacturer of a medicament for treating cancer, wherein the cancer is dependent on NSD3.
14. The use of paragraph 13, wherein the cancer is NUT midline carcinoma (NMC) characterized by the presence of at least one rearrangement of the NUT gene in a NMC tumor or cancer cell.
15. The use of paragraph 14, wherein the rearrangement in the NUT gene is a translocation of the NUT gene to form at least one of: a NSD3/NUT fusion gene, a BRD4/NUT fusion gene or a BRD3/NUT fusion gene.
16. A method of treating a subject with cancer, the method comprising assessing for the presence of the NSD3/NUT fusion oncoprotein or NSD3/NUT fusion gene in a biological sample obtained from the subject, wherein a clinician reviews the results and if the results indicate the presence of a NSD3/NUT fusion, the clinician directs the subject to be treated with a NSD3 inhibitor and/or a BET inhibitor.
17. A method of treating a subject with cancer, the method comprising assessing for the presence of the rearrangement of the NUT gene, wherein rearrangement of NUT to form a BRD4/NUT or BRD3/NUT fusion protein or gene in a biological sample obtained from the subject, wherein a clinician reviews the results and if the results indicate the presence of a BRD4/NUT or BRD3/NUT fusion, the clinician directs the subject to be treated with a NSD3 inhibitor.
18. The method of paragraph 16 or 17, wherein the biological sample is a tissue sample or biopsy sample.
19. A kit comprising a NSD3i and/or BETi and a pharmaceutically acceptable excipient, and optionally at least one additional cancer therapeutic to be co-administered with the NSD3i and/or BETi agent.
20. The method of any of claims 1 to 12, wherein the subject is selected for treatment based on having a NUT gene translocation, wherein the subject is identified to have a NUT gene that forms at least one of: a NSD3/NUT fusion gene, a BRD4/NUT fusion gene or a BRD3/NUT fusion gene.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

### EXAMPLES

The examples presented herein relate to the NSD3 inhibitors and/or BET inhibitors for NSD3-depdent cancers, e.g., NMC, or cancers characterized by the rearrangement of the NUT gene to form a NSD3/NUT fusion protein. In some embodiments, NSD3 inhibitors can also be used for cancers characterized by BRD4/NUT or BRD3/NUT rearrangement or for BRD4-dependent, NUT-independent cancers. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Materials and Methods

Fluorescence In Situ Hybridization

Dual-color fluorescence in situ hybridization (FISH) assays for NSD3 and NUT breakpoints were performed on formalin fixed, paraffin-embedded, 4 mm tissue sections as described (20). Probes used for the 15q14 breakpoint flanking NUT included the telomeric BAC clones RP11-1H8 and RP11-64o3, and the centromeric clones RP11-1084A12 and RP11-368L15. Probes used for the chromosome 8p11.23 NSD3 breakpoint were the flanking 5′ centromeric BAC clones CTD-2538P2 and RP11-957P17 and the 3′ telomeric BAC clones CTB-497A2 and RP11-90P5. Cytogenetic analysis and metaphase FISH was performed using standard methods (48) (49).

RNA-Sequencing

RNA was extracted from live cultured 1221 cells using the RNeasy mini kit (Qiagen). Elim Biopharmaceuticals (Hayward, Calif.) performed the library preparation and sequencing. rRNA removal was performed using the Ribo-Zero kit (Epicentre, Madison, Wis.) following the manufacturer's instructions. The library was prepared using standard Illumina protocols with proprietary modifications and sequenced using HiSeq2500 (Illumina, San Diego, Calif.). TopHat-Fusion (v2.0.8b bundled with TopHat2) was run with default parameters (as described at http://tophat.cbcb.umd.edu/fusion_tutorial.html but with -r 50 and --max-intron-length 1000000) to identify novel fusion transcripts from paired-end 50 base reads (50) (51).

Reverse-Transcriptase PCR (RT-PCR)

RT-PCR was as described (6) using the following primers:

NSD3f1382
(SEQ ID NO: 14)
AAGAGCCACCGCCTGTTAAA,
NUTr388
(SEQ ID NO: 54)
GCTGTCACAAATGGAGGTGC,
GAPDH254f
(SEQ ID NO: 55)
TCAAGTGGGGCGATGCTGGCGCT,
GAPDH788r
(SEQ ID NO: 56)
AGGGGGCCCTCCGACGCCTGCT.

Plasmids

BRD4 ET domain containing fragment (BRD4 444-722) was cloned into pcDNA5 FRT/TO-FLAG (Invitrogen) with an N-terminal SV40 NLS sequence to generate pcDNA5 FRT/TO-Flag-NLS-BRD4-ET (p6894). MSCV-CMV-Flag-HA-NSD3 (p6351) has been described previously (8). To make tetracycline-inducible N- and C-terminal BioTAP-tagged constructs, the inventors transferred the gateway destination cassette from pHAGE-TRE (gift of Steven Elledge) to the pcDNA5 frt/to mammalian expression vector with N-terminal BioTAP tandem tag to make pcDNA5 frt/to-DEST-NBioTAP. NSD3-NUT was constructed by fusion PCR into pDONR223, then transferred into the pcDNA5 frt/to-DEST-NBioTAP vector by gateway cloning. Full length NSD3, NSD3Tr encoding amino acids 1-569 of NSD3, full length NUT, BRD4-NUT (derived from pcDNA5 frt/to FLAG-BRD4-NUT (17)) were PCR-cloned into pDONR223, then transferred by gateway cloning into the pHAGE-P CMVt N-HA GAW expression vector derived from the PHAGE lentiviral vector (52).

A tetracycline-inducible HA-tagged NSD3Tr was gateway cloned from pDONR223 into the tetracycline-inducible pHAGE-P CMVt N-HA GAW expression vector and into the tetracycline-inducible pHAGE-TRE-HA (gift of Steven Elledge).

Cell Culture

NMC cell lines, TC-797 (15) 10-15 (6), 8645(17), 293T, U205, and C33A cells were maintained as monolayer cultures in Dulbecco Modified Eagle medium (DMEM) (Invitrogen) supplemented with 10% (v/v) Fetal Bovine Serum (FBS, SH3008803, Hyclone) and 1% pen-strep (GIBCO/Invitrogen). The 797TRex cell line was created using Flip-in technology as described (Invitrogen (6)) and maintained as above, but with the addition of Hygromycin (150 ug/ml, Sigma Aldrich, St. Louis, Mo.) and Blasticidin (7.5 ug/ml, Life Technologies, Grand Island, N.Y.) to maintain selection of cDNA insert and tet repressor genes, respectively. 797Trex/Flag-NLS-ET and N-BioTAP-NSD3-NUT cell lines were generated by recombination with the plasmid pcDNA5 FRT/TO-FLAG-NLS-ET and -N-BioTAP-NSD3-NUT (above) using Flp-In technology (Invitrogen). The resulting cell line was maintained in Dulbecco Modified Eagle medium (DMEM) (Invitrogen) supplemented with 10% (v/v) Fetal Bovine Serum (FBS, SH3008803, Hyclone), 1% pen-strep (GIBCO/Invitrogen), 7.5 ug Blasticidin/mL and 150 ug Hygromycin/mL. The 1221 cell line is derived from a lung metastasis from the index case of a 13 year old female with NSD3-NUT-positive NMC. The 1221 cells were grown and maintained in WIT media as described (17, 53). Tet-inducible C33A cells (C33A-6TR) cells were established by transfecting C33A cells with pcDNA6/TR vector (Invitrogen). Cells were selected and maintained with 5 μg/ml Blasticidin and single cell clones were obtained. Single cell clones were then tested for leakiness using a Tet-inducible EGFP construct and clone 13 was chosen for further experiments due to a tight regulation of EGFP expression by the TR. The 1221, TC-797, 797TRex, 8645, and 10-15 cell lines were authenticated by FISH (above) demonstrating rearrangement of the NUT, BRD4, and/or NSD3 genes. The C33A cell line has been authenticated by documentation of p53 and pRb mutations (54). Neither the 293T or U2OS cell lines have been authenticated.

Luminescent Cell Viability Assay

Cells were plated at a density of 3000 per well in a 96-well plate, and CellTiter-Glo (Promega, Madison, Wis.) was used to determine cell viability as a measure of ATP content according to the manufacturer's instructions.

siRNA Transfections

For TC-797, Per403, and 8645 cells, 7×10⁶ cells were transfected with 50 nM siRNA using Nucleofector II (Lonza, Basel, Switzerland) and Amaxa solution R and plated in 100 mm cell culture dish. 1221 cells were transfected using RNAi-MAX (Invitrogen) using the manufacturer's protocol. Briefly, reverse transfection procedure was used to deliver 50 nM siRNA to 33×10⁴ cells in a 6 well plate. Cells were analyzed for mRNA levels 24 h after transfection. Sequences of siRNA used were: siControl, ON-TARGET plus siRNA #1 (Dharmacon, Cat # D-001810-01-20), siNUT-1 (targeting coding sequence) AAACUCAGAACUUUAUCCUUAUU (SEQ ID NO: 57), siNUT-2 (targeting the 3′ UTR) UUACCUUUGGAAGGAGCUA (SEQ ID NO: 58), siBRD4 5′ siGENOME Human BRD4 (Dharmacon Cat # D-004937-02), siBRD4 3′ GGGAGAAAGAGGAGCGUGAUU (SEQ ID NO: 59), siNSD3-6 ON-TARGETplus Human WHSC1L1 (54904) (Dharmacon Cat # J-012875-06), siNSD3-7, ON-TARGETplus Human WHSC1L1 (54904) siRNA (Dharmacon Cat # J-012875-07), siNSD3 3′-1 CUGUAAACCUCUAAAGAAAUU (SEQ ID NO: 15), si NSD3 3′-2 GAAAGGUGCCAGCGAGAUUUU (SEQ ID NO: 16), siJMJD6-12 GGUAUAGGAUUUUGAAGCA (SEQ ID NO: 60) (Dharmacon Cat # J-010363-12-0020), siJMJD6-13 GGAUAACGAUGGCUACUA (SEQ ID NO: 61) (Dharmacon Cat # J-010363-13-0020), siNSD3-06 GAACGUGCUCAGUGGGAUA (SEQ ID NO: 17) (Dharmacon Cat # J-J-012875-06-0020), siNSD3-07 GCUUGAGGUUCAUACUAAA (SEQ ID NO: 18) (Dharmacon Cat # J-012875-07-0020), siGLTSCRI-05 GUAAUGAUCGACCGAAUGU (SEQ ID NO: 62) (Dharmacon Cat # J-020751-05-0020), siGLTSCR1-08 CCACCACGUUCAAUGGGAA (SEQ ID NO: 63) (Dharmacon Cat # J-020751-08-0020), siATAD-05 UAGCAGAAAUGUACAACUA (SEQ ID NO: 64) (Dharmacon Cat # J-004738-05-0020), siATAD5-06 GCGCAAUAAUGUAUACUUU (SEQ ID NO: 65) (Dharmacon Cat # J-004738-06-0020), siCHD4-07 (Dharmacon Cat # J-009774-07-0020), siCHD4-08(Dharmacon Cat # J-009774-08-0020).

Immunofluorescence

Immunofluorescence on TC-797 cells was performed as described (55) and nuclei were counterstained with ProLong® Gold antifade reagent with 4′,6-diamidino-2-phenylindole (DAPI) (Life Technologies). Primary antibodies used were anti-NUT (1:1000, rabbit monoclonal clone C52, Cell Signaling Technology), and anti-HA (1:500, mouse monoclonal, Sigma-Aldrich). Secondary antibodies included goat anti-rabbit Alexa Fluor 594 and goat anti-mouse Alexa Fluor 488 (1:1000, Life Technologies, Grand Island, N.Y.). Photos were taken on a Nikon Eclipse E600 fluorescent microscope (Melville, N.Y.) using a Spot RTSlider camera (Diagnostic Instruments, Inc., Sterling Heights, Mich.), and Spot Advanced software (Diagnostic Instruments, Inc.).

Quantitation of BRD4-NUT foci was performed by analyzing immunofluorescent images with ImageJ software. BRD4-NUT foci were identified by equal threshold adjustment of all images. The Analyze Particles function in ImageJ was used to determine foci number for each cell. 40 cells were counted for each experimental condition and experiments were performed in triplicate. Statistics shown in all figures are from Student's t-Tests (two tailed).

High Throughput Immunofluorescence Analysis of NMC Cells

Cells were transfected in 384 well format using 50 nM control siRNA (above), NSD3 siRNA (above), BRD4 siRNA, and NUT siRNA (above), as described (6), or a dose range of JQ1 treatment. Cells were stained with AE1/AE3 antibody (1:4, Dako, Carpinteria, Calif.) to measure keratin intensity, Ki-67 antibody (1:500, Cell Signalling Technology, Danvers, Mass.), and nuclei were stained with Hoechst 33342 (4 ug/ml, Molecular Probes). Imaging for keratin expression was performed using the ImageXpress high-throughput microscope with MetaXpress software (Molecular Devices, Sunnyvale, Calif.) as described (56). Shown are representative images taken at 40× magnification. Each condition was performed in triplicate in 384-well plate format in three separate experiments, where wells were analyzed using MetaXpress with Multi Wavelength Cell Scoring for cell number, average keratin fluorescence pixel intensity, and % Ki-67 per well. Statistics shown are from Student's T-tests.

Gene Expression Analysis Using Quantitative Real-Time PCR (qRT-PCR)

Total RNA was harvested at the indicated time points using TRizol (Invitrogen, Carlsbad, Calif., USA) and the RNeasy Mini Kit (Qiagen, Valencia, Calif., USA) and RNA (1 ug) was reversed transcribed into cDNA using the iScript cDNA synthesis kit (Bio-Rad). Quantitative RT-PCR was performed as described (6) using the reference gene Ribosomal protein L13a (RPL13a) mRNA levels as normalizing control. All qRT-PCR experiments are representative of triplicate qRT-PCRs from one of three independent experiments. qRT-PCR was performed in triplicate on a Bio-Rad iCycler in 96 well plate format with IQ SYBR Green supermix (Bio-Rad) and 1 ul of cDNA template per reaction. Amplification curves and Ct values were generated using MyiQ Single-Color Real-Time software (Bio-Rad). Primers used were 3′ NSD3 fwd TTCTAGGAGTGCGGCCAAAG (SEQ ID NO: 66), 3′ NSD3r CAGCTCTCCACCATCTCCAC (SEQ ID NO: 67), 5′ NSD3f GCCCCAGTTCAGCCAATACT (SEQ ID NO: 68), 5′ NSD3r ACCATACAAGGCCACCAAGG (SEQ ID NO: 69), RPL13Af CCTGGAGGAGAAGAGGAAAGAGA (SEQ ID NO: 70), RPL13Ar TTGAGGACCTCTGTGTATTTGTCAA (SEQ ID NO: 71), and using the TaqMan primers (Life Technologies, Grand Island, N.Y.), JMJD6 (Hs00397095_m1). NSD3 (Hs00256555_m1). GLTSCR1 (Hs00185249_m1), and ATAD5 (Hs00227495_m1).

Immunoblotting

Cell lysis and immunoblotting was performed as described (17). Antibodies used were rabbit anti-NUT (1:500, AX.1 rabbit polyclonal antibody (57), anti-GAPDH (1:5000, mouse monoclonal 6C5, Life Technologies, Grand Island, N.Y.), anti-BRD4 (1:1000, Bethyl Laboratories Inc., Montgomery, Tex.), anti-FLAG (1:1000, mouse monoclonal, Sigma-Aldrich, St Louis, Mo.), anti-involucrin (1:1000, mouse monoclonal, Sigma-Aldrich), anti-HA (1:1000, mouse monoclonal, Sigma-Aldrich), anti-NSD3 (1:500, mouse monoclonal clone 2E9, Lifespan Biosciences, Seattle, Wash.), anti-peroxidase anti-peroxidase complex/PAP antibody (1:5000, rabbit polyclonal, Sigma-Aldrich), anti-histone H3 (1:1000, mouse monoclonal ab10799, Abacam), anti-actin (1:1000, mouse monoclonal clone 4, Millipore) and anti-p300 (1:1000, mouse monoclonal clone RW128, EMD Millipore, Billerica, Mass.).

Immunoprecipitation

For co-immunoprecipitation experiments of HA-tagged proteins, C33A-6TR cells stably expressing HA-tagged constructs under a Tet-inducible promoter were used. Protein extracts were prepared 24 h after transfection or induction with Doxycycline (1 μg/ml). Cells were lysed in lysis buffer (50 mM Tris-HCl [pH 7.5], 150 mM NaCl, 0.5% Nonidet P-40) with freshly added protease inhibitors (Roche Complete, EDTA free protease inhibitor cocktail, Roche, Indianapolis, Ind.). Extracts were adjusted for protein concentration and 10% of extracts were used as Input Immunoprecipitations were performed using 15 μl of HA-resin (Sigma A2095). Extracts were incubated overnight at 4° C., and precipitated proteins were detected by Western blot analysis.

Immunohistochemistry

Formalin-fixed, paraffin-embedded cell-blocks of cultured cells were prepared using Histogel (Richard-Allan Scientific) as described previously (2). Sections were stained with hematoxylin and eosin or by immunohistochemistry (IHC), which was performed on 5 mm sections prepared from formalin-fixed, paraffin-embedded cell-blocks Immunohistochemical stains were performed using anti-NUT antibody (1:100, rabbit monoclonal clone C52, Cell Signaling Technology, Danvers, Mass.), anti-involucrin antibody (1:12000, Sigma-Aldrich, St. Louis), and Ki-67 (MIB-1 clone; DAKO USA, Carpinteria, Calif. (2). One hundred cells were counted per sample for Ki-67 percentage. Standard deviations for triplicate counts are shown in figures.

### Example 1

### A Novel NSD3-NUT Fusion in NUT Midline Carcinoma

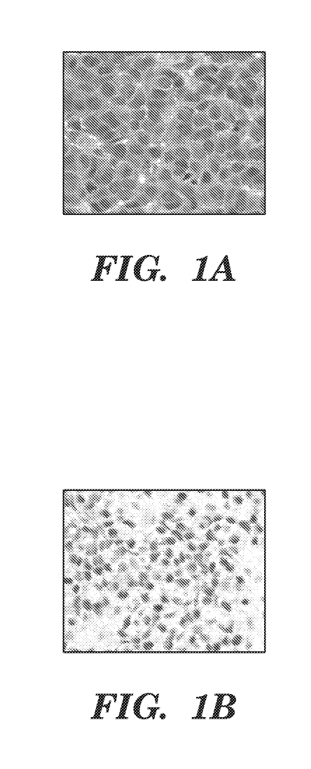

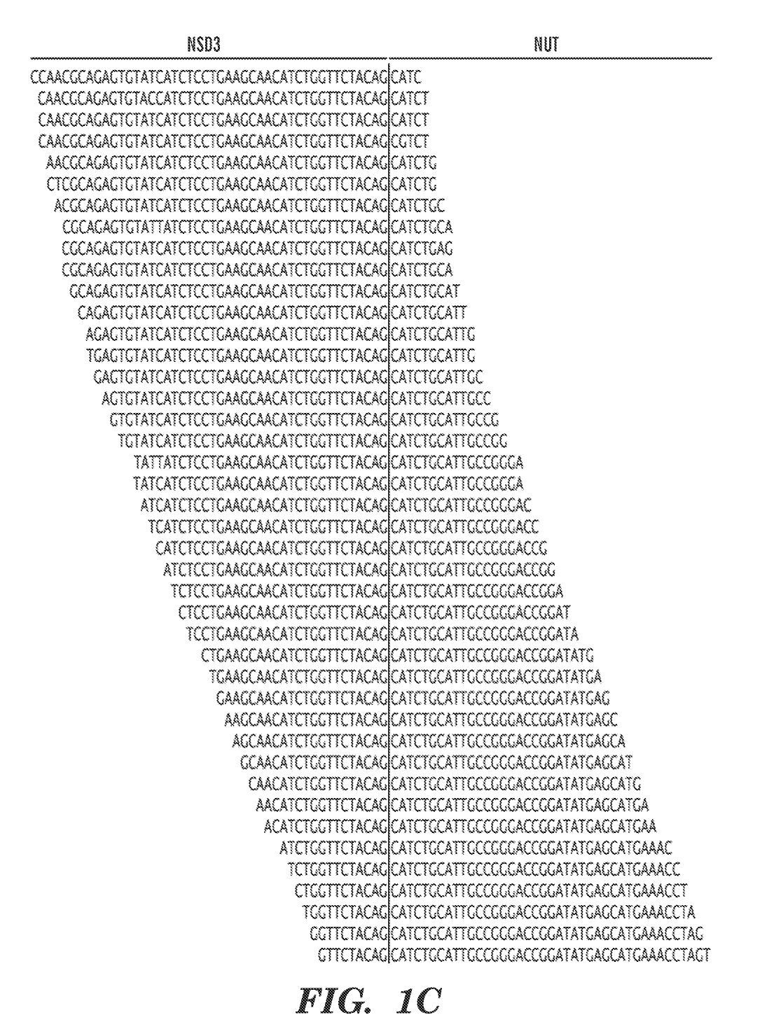

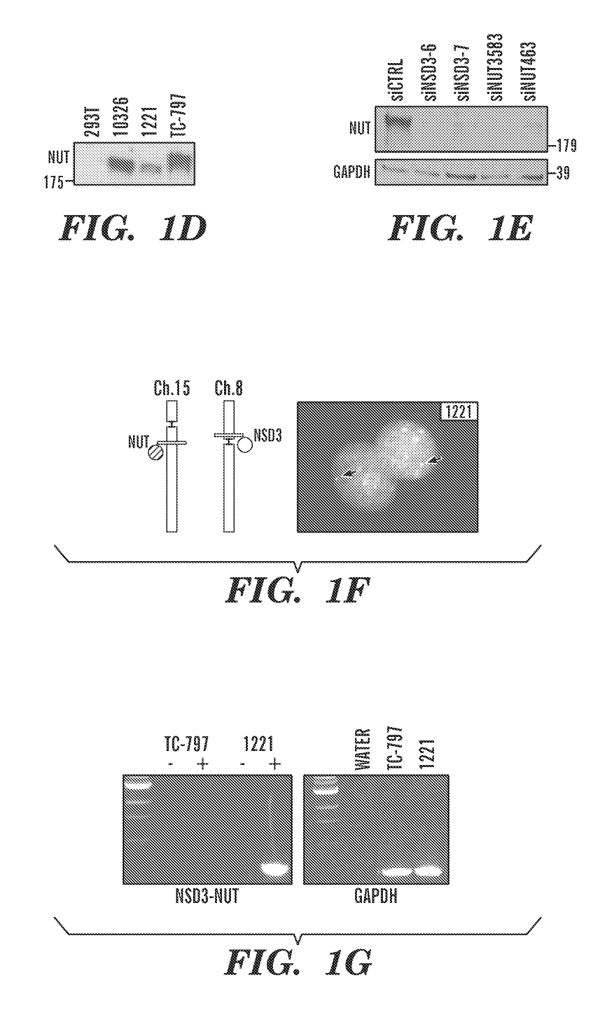

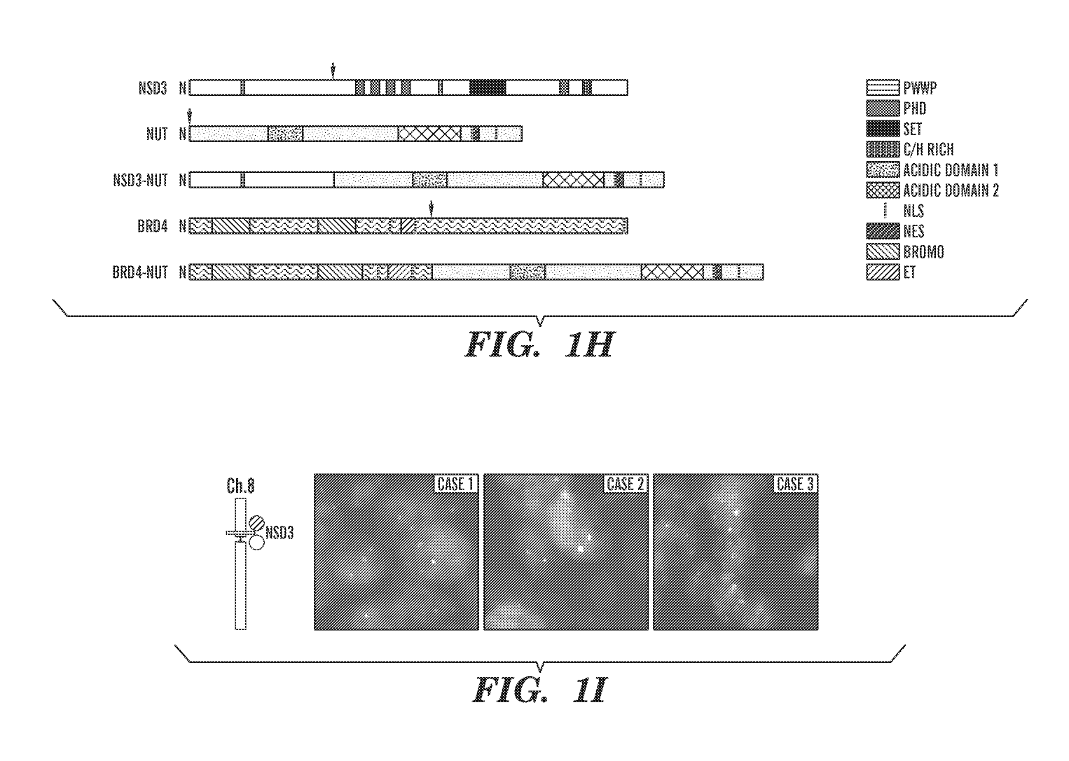

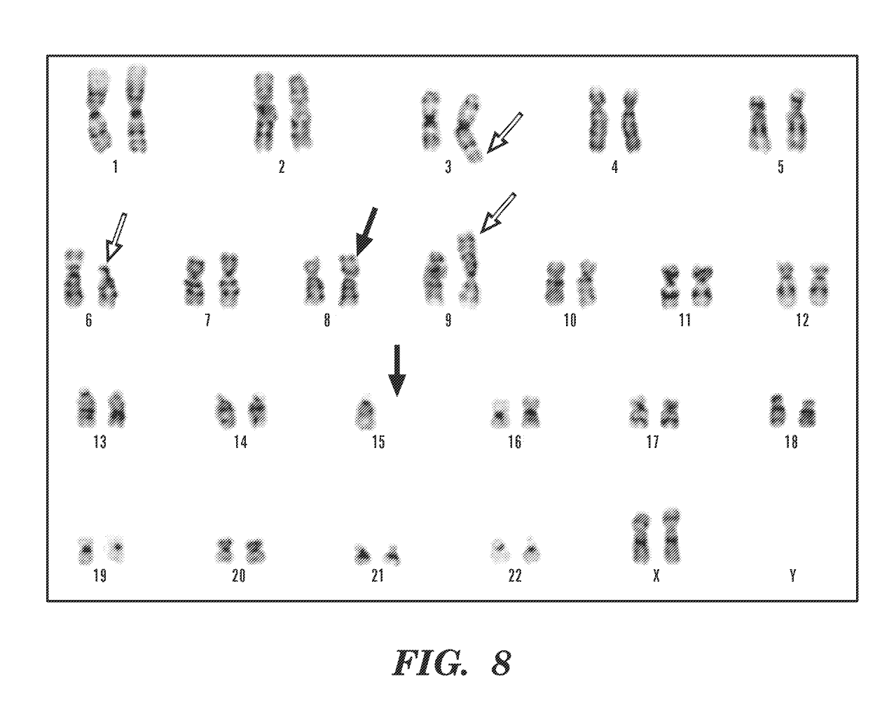

A poorly differentiated squamous cell carcinoma of the mediastinum (FIG. 1A) metastatic to the femur of a 12 year old girl was referred to us for molecular diagnostic testing for NUT midline carcinoma Immunohistochemical analysis revealed diffuse nuclear expression of the NUT protein, a feature that is diagnostic of NMC (FIG. 1B (10)). Fluorescent in situ hybridization (FISH) demonstrated rearrangement of the NUT gene locus on chromosome 15q14, however neither BRD4 nor BRD3 rearrangement were detected. Discarded live tumor tissue from a metastatic focus in the patient's lung was collected under institutional review board approval through the NUT midline carcinoma registry (www.NMCRegistry.org). From this tissue the first known NUT-variant cell line, 1221, was established. To determine the putative partner gene to NUT, the inventors performed comprehensive RNA-sequencing on RNA purified from 1221. The inventors identified an in-frame transcript fusing the 5′ coding sequence of NSD3 (exons 1-7) to exons 2-7 of NUT (FIG. 1C). Expression of the NSD3-NUT fusion oncoprotein was verified by immunobloting with an antibody to NUT, revealing an approximately 200 kDa band that is similar in size to BRD3-NUT, but smaller than BRD4-NUT (FIG. 1D). Knockdown using siRNAs targeting NSD3 led to a disappearance of the putative NSD3-NUT band, as did siRNAs targeting NUT, confirming the identity of the NSD3 and NUT portions of the NSD3-NUT fusion protein (FIG. 1E). Genomic fusion of the NSD3 and NUT genes was confirmed by FISH, demonstrating bring-together of NUT and NSD3 probes (FIG. 1F). Likewise, the expression of an NSD3-NUT mRNA was demonstrated by reverse transcriptase PCR (RT-PCR, FIG. 1G). Cytogenetic analysis of the 1221 cell line was consistent with a NSD3-NUT fusion, revealing a t(8;15)(p12;q15) translocation, and metaphase FISH demonstrated localization of the NUT probe near the NSD3 chromosomal region (8p11.23) (FIGS. 8-9). Several additional aberrations of unknown significance were also present.

The fusion sequence is predicted to encode a 1694 amino acid protein containing amino acids 1-569 of NSD3, and 8-1132 of NUT. Interestingly, the NSD3 portion of the fusion protein lacks the SET domain and contains only its PWWP domain (or Pro-Trp-Trp-Pro motif) (SEQ ID NO: 13), whereas nearly all of NUT is included in the fusion, as is typical in NMC (1, 11, 12) (FIG. 1H). The NSD3-NUT fusion bears no resemblance to NUP98-NSD3/NSD1 fusion oncogenes that have been previously described in leukemia (13, 14), all which fuse NUP98 to the 3′ end of NSD3/NSD1 containing their SET, PHD, and C/H rich domains.

NSD3-NUT is a Recurrent Form of NMC

The inventors next sought to determine whether NSD3 is a recurrent NUT-fusion partner in NMCs, thus performed a dual color NSD3 split-apart FISH assay on several NUT-variant cases. Four of eight non-BRD3/BRD4-NUT NMC cases (including the index case) demonstrated rearrangement of NSD3 and NUT, suggesting a frequent incidence of NSD3-NUT amongst NUT-variant cases (FIG. 1I).

NSD3-NUT is Required for the Blockade of Differentiation and Maintenance of Proliferation in 1221 NMC Cells

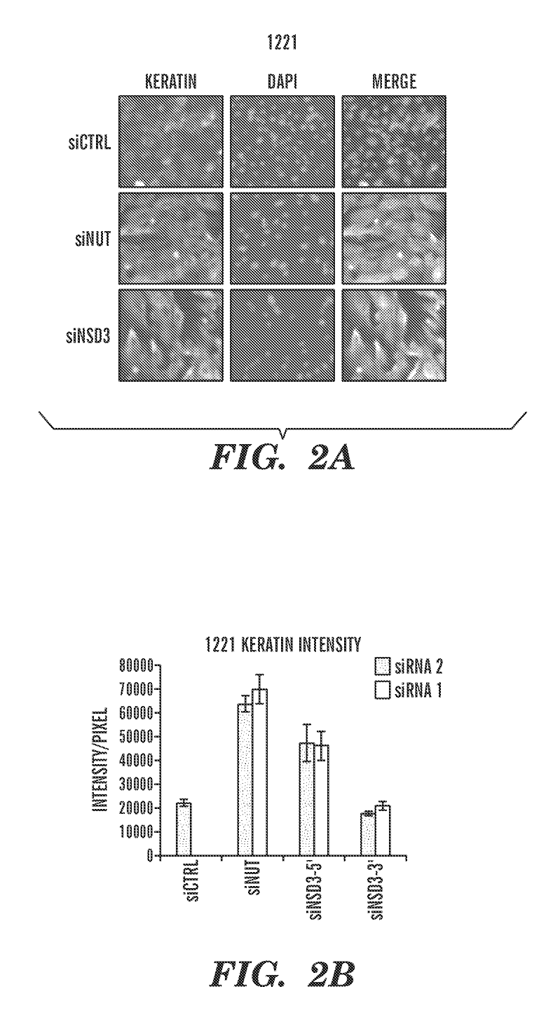

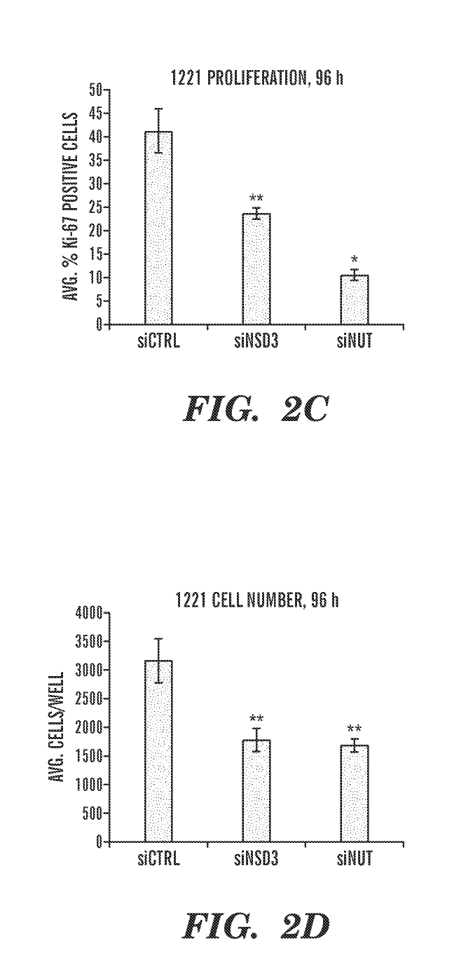

The recurrent existence of NSD3-NUT in NMCs suggested that it may function similarly to BRD-NUT by blocking differentiation and maintaining proliferation of NMC cells (2). The inventors knocked down endogenous expression of NSD3-NUT in 1221 cells to determine its effect on growth and differentiation. Seventy-two hours following knockdown, 1221 cells exhibited differentiation as evidenced by increased keratin expression, an epithelial differentiation marker, by immunofluorescence (FIG. 2A-B), and decreased proliferation as measured by Ki-67 fraction (FIG. 2C) and cell number (FIG. 2D). Notably, knockdown of wild type NSD3 using siRNAs directed toward the 3′ aspect of NSD3 that is not included in the NSD3-NUT fusion gene had no effect on differentiation (FIG. 2B). These discoveries demonstrate that NSD3-NUT serves to block differentiation and maintain proliferation of 1221 cells.

Wild Type NSD3 is Required for the Blockade of Differentiation in BRD4-NUT-Expressing NMC Cells

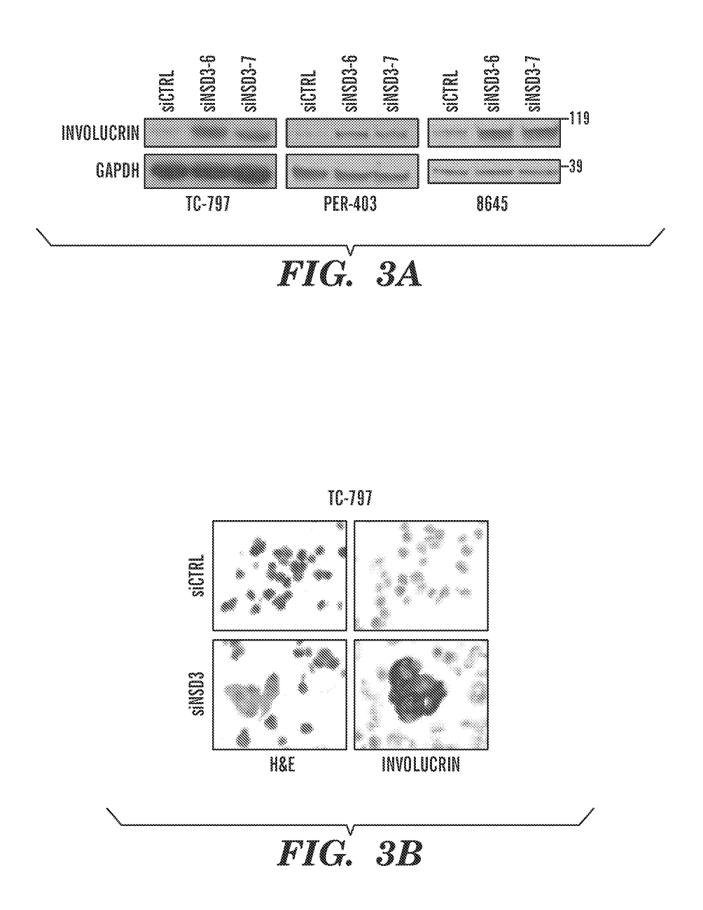

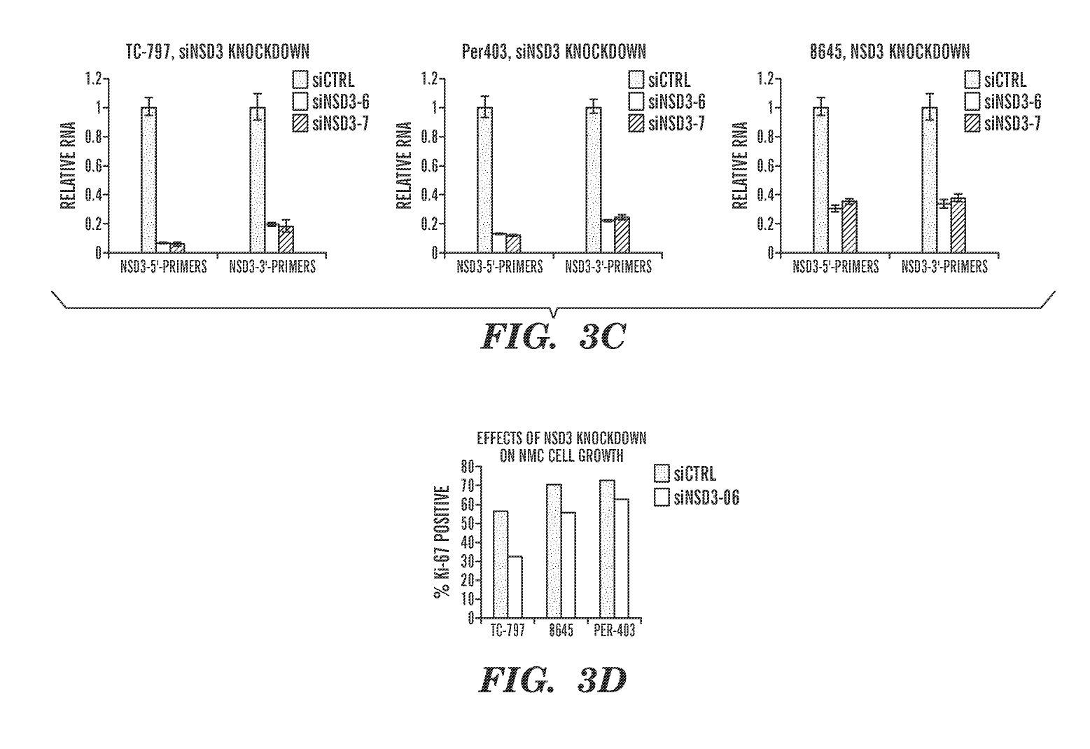

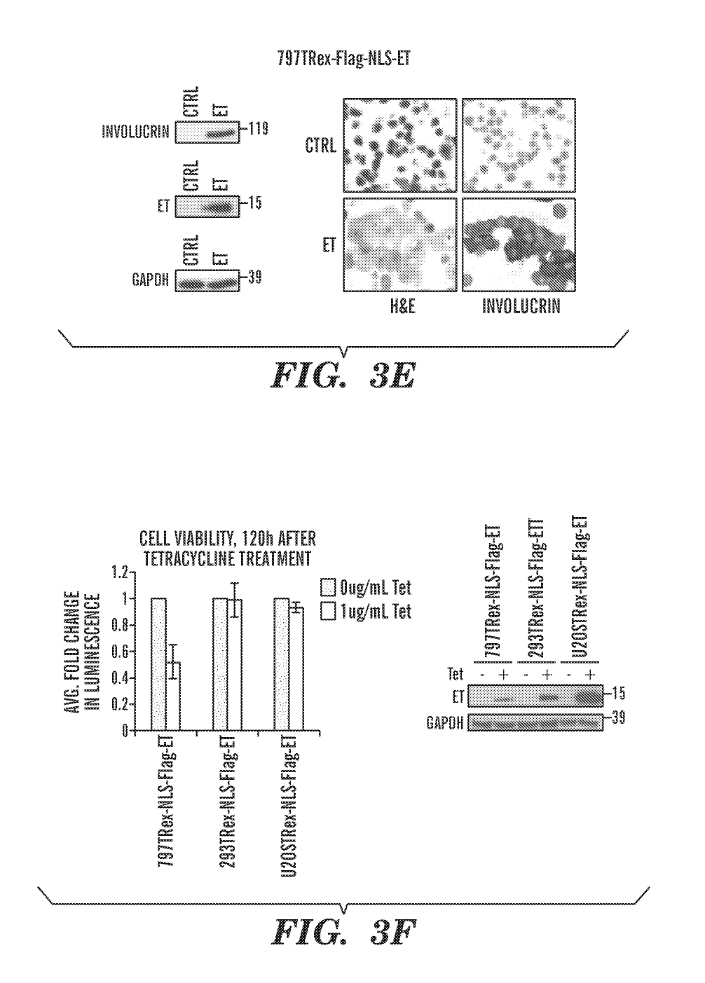

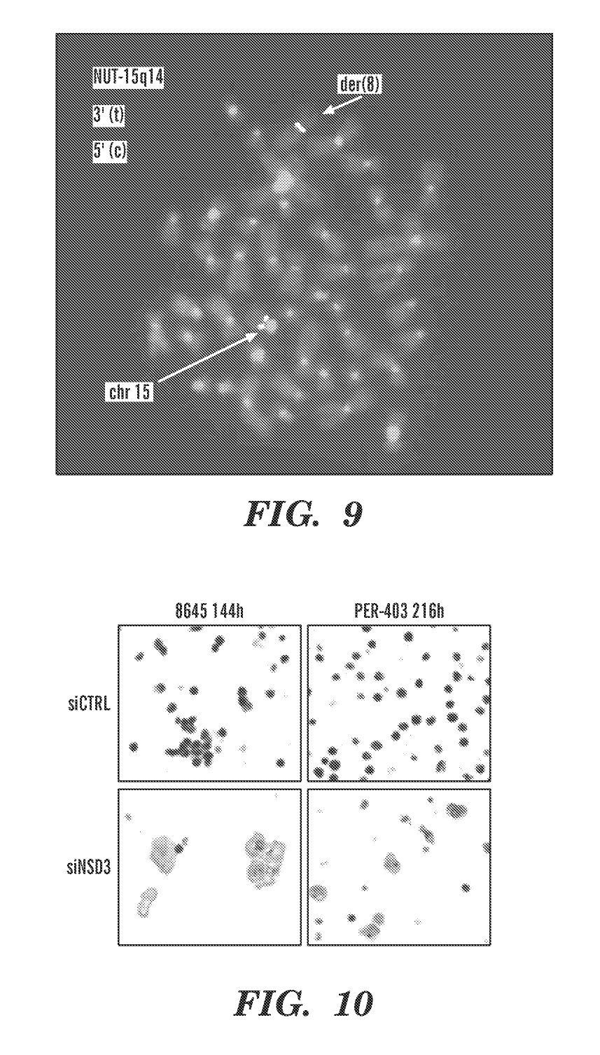

NSD3 is one of several proteins that have been shown to bind the ET domain of BET proteins, (8). Thus it was assessed if NSD3 may have an oncogenic role in NMC through its interaction with BRD4-NUT's retained ET domain. It is noted that the BRD3 and BRD4 fusions with NUT in the BRD-NUT NMCs occur 3′ to the ET domain, thus the ET domain is always included as part of the fusion protein(1, 2). The inventors therefore tested whether NSD3 is required for the blockade of differentiation in BRD4-NUT-expressing NMC cells. In three different patient-derived BRD4-NUT+ NMC cell lines, TC-797 (15), PER-403 (16), and 8645 (17), siRNA-knockdown of NSD3 resulted in differentiation, as measured by increased expression of the terminal squamous differentiation marker, Involucrin (FIG. 3A-C). This was accompanied by morphologic differentiation, as evidenced by flattening and enlargement of cells (FIG. 3B, and FIG. 10), as well as mild-to-moderate decreased proliferation quantified by Ki-67 staining (FIG. 3D) in all cell lines. Moreover, induced expression of the ET domain fused to a nuclear localization sequence (NLS) in a tet-inducible NMC derivative cell line, 797TRex, exhibited a dominant negative effect on BRD4-NUT function, inducing differentiation morphologically and immunophenotypically (FIG. 3E). In addition, induction of ET domain expression also negatively affected the proliferation rate of TC-797 cells, whereas the growth of heterologous, non-NMC cells, U2OS or 293T, was unaffected (FIG. 3F) This discovery demonstrates that expression of wild type NSD3 protein and the ET domain of BRD4-NUT are required for the blockade of differentiation in BRD4-NUT+ NMC. The specific requirement of the ET domain for the oncogenic function of BRD4-NUT is evidenced by its conservation in all characterized BRD-NUT fusions (1, 2, 12, 18, 19), including uncommon splice variants(16, 18), and by the lack of growth inhibition induced by ET domain expression in non-BRD-NUT-expressing cell lines (FIG. 3F).

### Example 2

### The N-Terminus of NSD3 Associates with BRD4 and BRD4-NUT

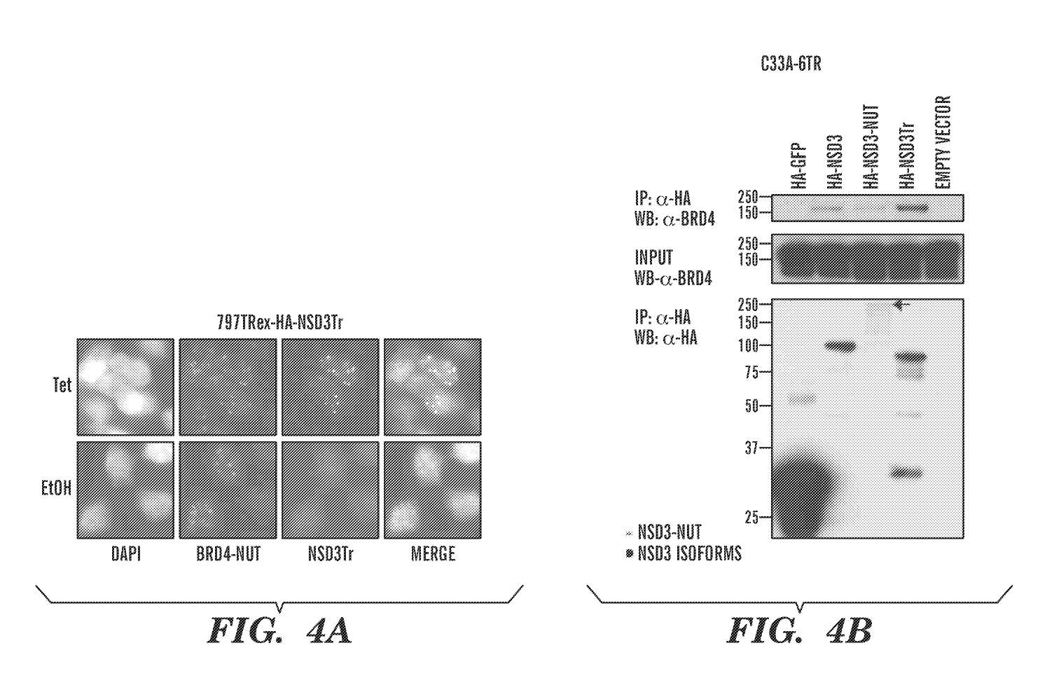

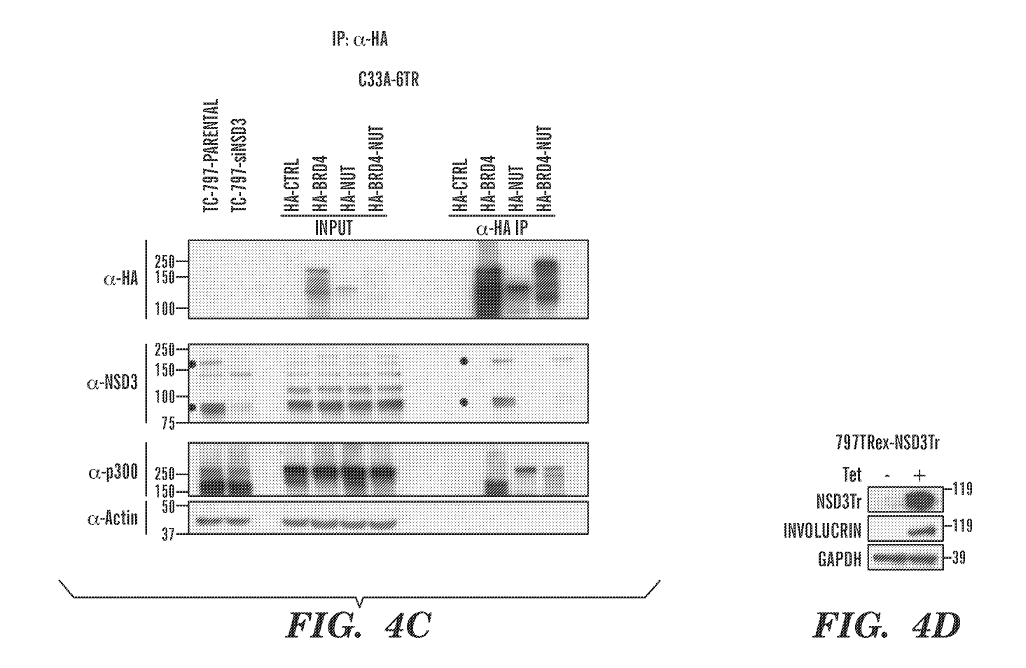

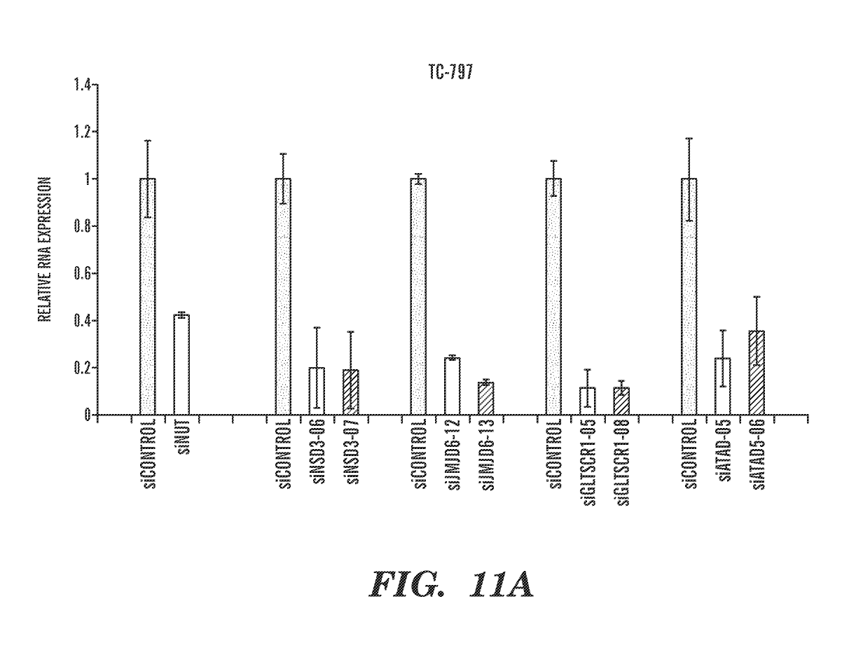

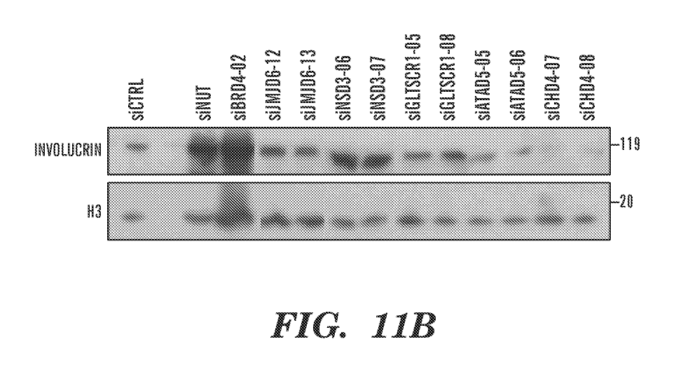

Because the ET domain is retained in BRD-NUT oncoproteins, the inventors assessed if the interaction of NSD3 with BRD4 would be preserved when co-expressed with BRD4-NUT. BRD4-NUT normally localizes to discrete nuclear foci by immunofluorescence and immunohistochemistry. The inventors discovered that the HA-tagged portion of NSD3 present in NSD3-NUT (NSD3Tr, corresponding to amino acids 1-569 of NSD3) co-localized with BRD4-NUT foci (FIG. 4A). Moreover, HA-tagged NSD3, NSD3-NUT, and NSD3Tr (FIG. 4B) co-immunoprecipitated BRD4 in C33A cervical carcinoma cells. In reciprocal experiments, HA-tagged constructs of BRD4 and BRD4-NUT, but not NUT, were able to co-immunoprecipitate NSD3 (FIG. 4C). Of note, the multiple NSD3 isoforms seen in this blot all contain the N-terminal domain of NSD3 (NSD3Tr) that is present in the NSD3-NUT fusion protein that interacts with BRD4 (FIG. 4B). This discovery demonstrates that NSD3 does associate with BRD4-NUT. To determine the role of the association of NSD3 with BRD4-NUT in the blockade of differentiation, NSD3Tr was expressed in 797TRex cells, and was found to induce differentiation (FIG. 4D). Thus, coupled with the dominant negative effects of ET domain expression (FIG. 3E-F), the inventors have demonstrated that the interaction of NSD3 with BRD4-NUT is required for the blockade of differentiation. In support of this, other known interactors of the ET domain, including CHD4, ATAD5, GLTSCR1, and JMJD6 (MCB), were knocked down in TC-797 cells, but failed to induce differentiation (FIGS. 11A-B).

NSD3 is Required for BRD4-NUT Foci Formation

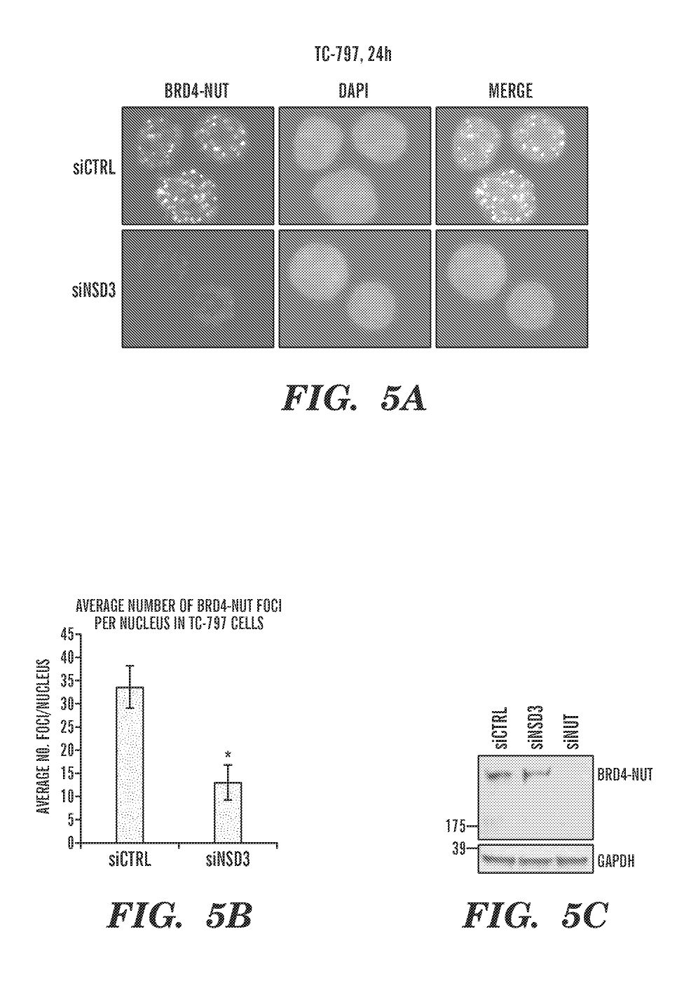

The function of BRD4-NUT foci is unknown; however, it has been demonstrated that they are intensely enriched with BRD4-NUT and factors associated with transcription, including RNA polymerase II, the histone acetyl-transferase (HAT), p300, the transcriptional elongation complex P-TEFb, and active histone marks (9, 20). Based on these observations, it is believed that BRD4-NUT foci are important to the function of BRD4-NUT on transcriptional regulation (9, 20), though this has not been tested directly. To determine what role, if any, NSD3 has in the formation of BRD4-NUT foci, the inventors knocked it down in TC-797 cells and quantified foci number as compared with control siRNA transfected cells. The inventors discovered that the number of BRD4-NUT foci was significantly reduced within 24 hours following siRNA knockdown of NSD3 (FIG. 5A-B). Reduction of BRD4-NUT foci post-NSD3 knockdown was not accompanied by a reduction of total BRD4-NUT protein levels (FIG. 5C), indicating that the effect is not due to loss of BRD4-NUT. Thus, this discovery demonstrates that NSD3 is required for BRD4-NUT focus formation, demonstrating a key role in BRD4-NUT complex or aggregate formation.

NSD3-NUT can Replace BRD4-NUT in the Blockade of Differentiation

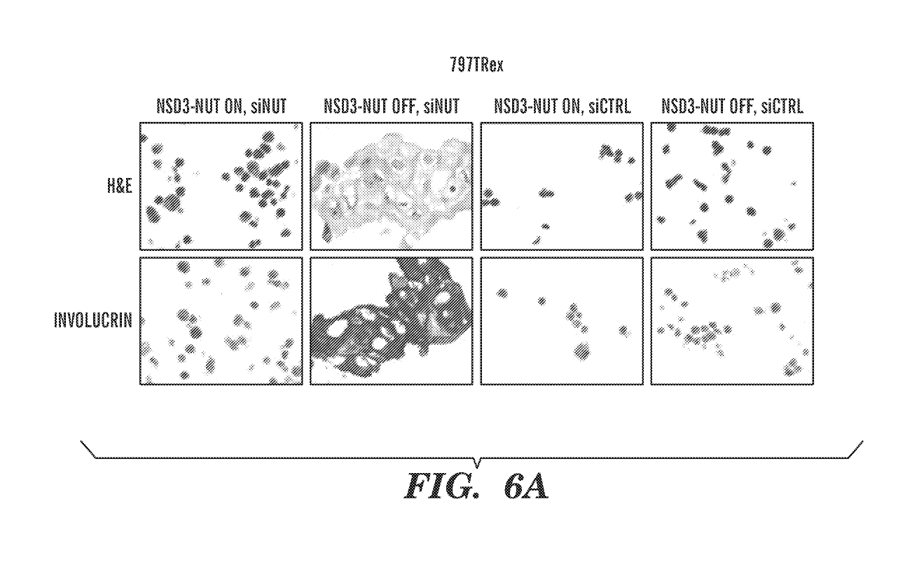

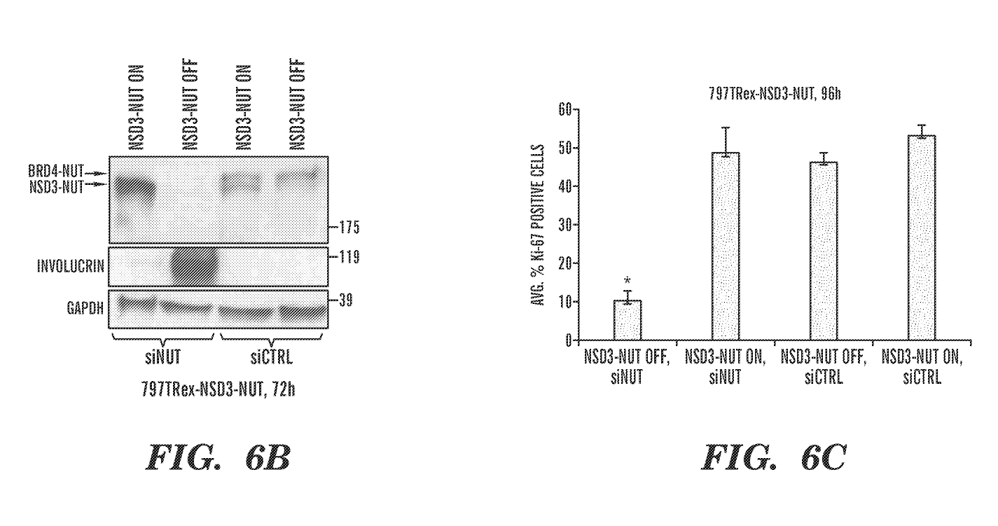

Because NSD3 is critical for BRD4-NUT function, the inventors assessed whether NSD3-NUT was functionally equivalent to BRD4-NUT and as such replaces BRD4-NUT's function in blocking differentiation. To test this, the inventors induced expression of a Bio-TAP tandem tagged NSD3-NUT construct with tetracycline in 797TRex cells subjected to BRD4-NUT knockdown using an siRNA targeting the 3′ untranslated region of NUT not present in the NSD3-NUT introduced gene. Indeed, induction of expression of NSD3-NUT in 797TRex cells almost completely abrogated differentiation and proliferation arrest in cells subjected to knockdown of endogenous BRD4-NUT. The ability of NSD3-NUT to block differentiation was demonstrated morphologically, showing a lack of flattening (FIG. 6A), and immunophenotypically, showing markedly reduced involucrin expression (FIG. 6A-B), as compared with cells not induced to express NSD3-NUT (ethanol vehicle control-treated cells). The ability of NSD3-NUT to maintain proliferation was demonstrated by a Ki-67 fraction similar to scrambled siRNA-transfected cells, and markedly greater than ethanol vehicle control-treated cells subjected to BRD4-NUT knockdown (FIG. 6C).

### Example 3

### BET Inhibitors Arrest Proliferation and Induce Differentiation of NSD3-NUT-Expressing NMC Cells

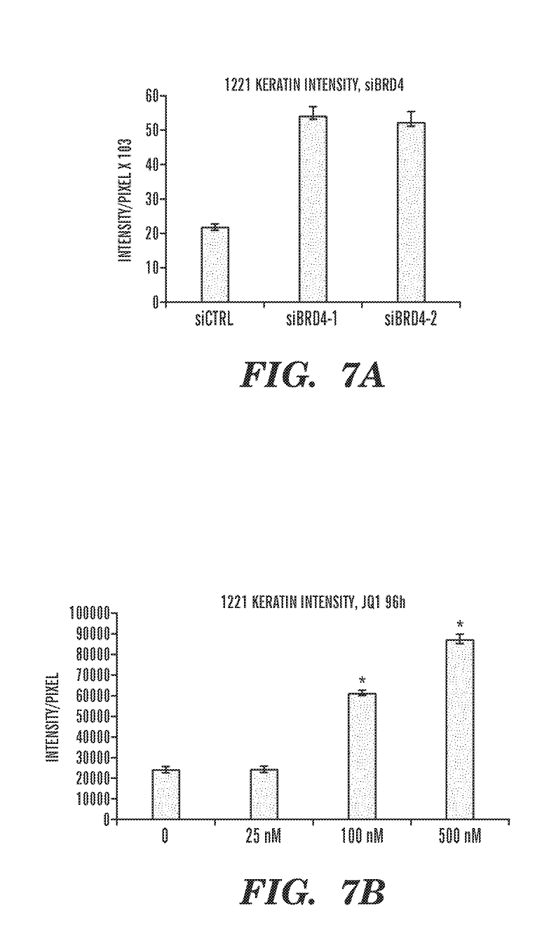

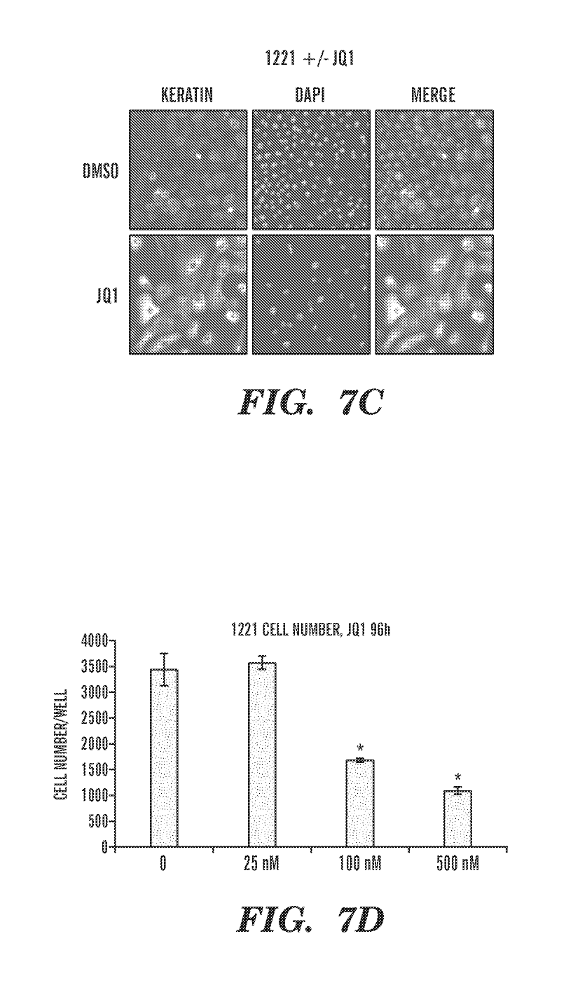

The existence of NSD3 as a NUT-fusion oncogene partner, whose encoded protein is also an important functional member of BRD4 and BRD4-NUT complexes, is reminiscent of the oncogenic mechanism of MLL-fusion associated leukemia (21). Thus, the inventors discovered that the oncogenic function of NSD3-NUT may depend on its interaction with BRD4 as a component of a chromatin-modifying complex with similar function to BRD4-NUT. Indeed, siRNA knockdown of BRD4, both long and short isoforms, induces differentiation of 1221 cells (FIG. 7A), and treatment of 1221 cells with the BET inhibitor, JQ1, results in differentiation and arrested proliferation, in a dose-dependent manner (FIG. 7B-D). Thus this discovery, together with the functional interchangeability of NSD3-NUT and BRD4-NUT, provide evidence that NSD3-NUT utilizes the chromatin-reading function of BRD4. Moreover, these data provide a sound rationale for treatment of patients with NSD3-NUT-positive NMCs using BET inhibitors.

### Example 4

### The Role of NSD3 in NMC

A number of recent variant translocations have been described in NMC, illustrating the heterogeneous nature of this disease (18, 22-24). In all of these previously described variants, both NUT and BET genes are fused. As described herein, the inventors have discovered a novel fusion gene in NMC that does not include a BET protein, but rather a BET-binding protein, NSD3. The inventors discovered that the NSD3-NUT fusion oncogene encodes a protein that is both necessary and sufficient for the blockade of differentiation in NMC. Importantly, the inventors also discovered that wild type NSD3, which binds to BRD4 in non-neoplastic cells, also binds to BRD4-NUT and is required for the blockade of differentiation by more common BRD4-NUT-expressing NMCs. The presence of a fusion oncoprotein involving constituents of a single oncogenic complex is well documented in cancer, and the existence of NSD3-NUT speaks to the importance of NSD3 and its association with BRD4-NUT. NSD3, also known as WHSC1L1, is a histone methyl-transferase that belongs to the mammalian Nuclear SET Domain-containing (NSD) protein family of SET domain-containing methyltransferases, which also includes NSD1 and NSD2 (WHSC1/MMSET). Both NSD3 and NSD2 are known to bind the ET domain of BRD4, thus the dominant negative phenotype of ET and NSD3Tr expression in NMC cells is evidence that this interaction of BRD4 with NSD3 may be critical to BRD4-NUT function. However, it is not clear that the methyltransferase activity of NSD3 is needed for BRD4-NUT function as it is for NUP98-NSD3 fusions (14), because the NSD3 portion of NSD3-NUT lacks the SET domain. Moreover, knockdown of full-length NSD3 in the 1221 cells (FIG. 2B) does not induce differentiation, indicating that NSD3's SET domain is not required for the differentiation blockade. Thus, it appears that the critical portion of NSD3 is its N-terminal, ET-binding domain in NSD3-NUT-expressing NMC. Combining this data with the fact that BRD4 expression and interaction with chromatin is required for NSD3-NUT function (FIG. 7), the inventors have discovered a model whereby the NSD3 portion links NSD3-NUT to BRD4, which tethers NSD3-NUT to chromatin, forming a complex that functions similarly to BRD4-NUT. In the context of BRD4-NUT-expressing NMC, the inventors have discovered that NSD3 is required for BRD4-NUT foci formation (FIG. 5A), demonstrating a role in the aggregation of large BRD4-NUT-containing complexes.

Although its importance is not known in NSD3-NUT+ NMCs, NSD3 methyltransferase activity may be important to the function of BRD4-NUT NMC. The SET domain of NSD proteins is homologous to the Saccharomyces cerevisae histone 3 lysine 36 (H3K36) specific methyltransferase SET2 and is specific for H3K36 dimethylation (H3K36me2) (25). NSD3 has been reported to regulate H3K36 methylation and thereby active gene expression (8, 26). It is possible that akin to the aberrant activation of HoxA1 expression by NUP98-NSD1-mediated methylation of H3K36 in acute leukemia (14), NSD3 may contribute to the transcriptional activation of key targets of BRD4-NUT. Although the NUP98-NSD3 fusion oncogene has been described in acute leukemia as well (13), it has not been further characterized mechanistically. Proteomic analysis of NSD3 indicates that the protein also interacts with the histone protein variant, macroH2A1. MacroH2A1 replaces conventional H2A histones in a subset of nucleosomes, where it represses or activates transcription and participates in stable X chromosome inactivation (27-29). Moreover, macroH2A1 regulates cell growth and differentiation and is differentially expressed in cancer cells (30-32). Thus, NSD3 may regulate differentiation in BRD4-NUT expressing NMC cells by affecting differentiation specific genes via alterations of H3K36 or macroH2A1 levels.

BET and NSD3 Proteins in Cancer

NSD family proteins have been associated with other cancers (Reviewed in (33)). Chromosome translocation resulting in NSD2 (aka MMSET) overexpression leads to multiple myeloma (MM) whereas reduction in NSD2 levels suppresses cancer growth (34-36). Moreover, NSD3 is amplified in breast cancer cell lines and primary breast carcinomas (37, 38). NSD3 has been reported to contribute to the transformed phenotype and invasiveness of these breast cancer cells (39, 40). Most recently, mutations in NSD3 have been identified in pancreatic adenocarcinoma (41). Despite these associations, the mechanism by which NSD3 contributes to oncogenesis in these cancers remains poorly understood.

A key to understanding the role of NSD3 in cancer may be through its association with BRD4. The indispensability of the BRD4 chromatin-binding bromodomains in NMC (4, 6) and its ET domain's role in recruiting NSD proteins demonstrates that BRD4 is a key player in BRD4-NUT chromatin-associated oncogenic complex formation. Moreover, recent studies with BET inhibitors have shown that BRD4 plays a role in other human cancers such as acute myeloid leukemia, multiple myeloma, and Burkitt lymphoma (42-44). In these cancers, as well as in NMC, BRD4 and BRD4-NUT, respectively, are required for the maintenance of MYC expression, and BET inhibitors repress expression of MYC, presumably through interference with BRD4-chromatin interaction (6, 43, 44). The inventors discovery that siRNA knockdown of NSD3 or BET inhibitor blockade of BRD4 function induces differentiation in NSD3-NUT-expressing NMC cells indicates that NSD3 function depends on BRD4 and its interaction with chromatin (FIG. 7). If NSD function is dependent upon BRD4 in other cancers, these NSD-associated cancers may be responsive to BET inhibitor therapy. Thus oncogenic NSD may be a biomarker of response to BET inhibitor therapy.

Drug Targeting of NSD3

Apart from BET inhibitors, this study highlights the importance of the therapeutic potential of targeting the NSD family of proteins. Histone modifying enzymes, including the NSD family, are often deregulated in cancer and aberrant histone modification profiles are intimately linked to carcinogenesis (45). Histone deacetylase (HDAC) inhibitors have been found to be effective in inhibiting NMC (17) and are already approved by the FDA for certain leukemias, while inhibitors to histone methyltransferases, including the NSD family, are under development (33, 45-47). Such therapies hold promise for NUT midline carcinoma as well as other cancers.

### REFERENCES

The references cited herein and throughout the application are incorporated herein in their entirety by reference.
1. French C A, Miyoshi I, Kubonishi I, Grier H E, Perez-Atayde A R, Fletcher J A. BRD4-NUT fusion oncogene: a novel mechanism in aggressive carcinoma. Cancer research. 2003; 63(2):304-7.
2. French C A, Ramirez C L, Kolmakova J, Hickman T T, Cameron M J, Thyne M E, et al. BRD-NUT oncoproteins: a family of closely related nuclear proteins that block epithelial differentiation and maintain the growth of carcinoma cells. Oncogene. 2008; 27(15):2237-42. Epub 2007/10/16.
3. Bauer D E, Mitchell C M, Strait K M, Lathan C S, Stelow E B, Luer S C, et al. Clinicopathologic Features and Long-Term Outcomes of NUT Midline Carcinoma. Clinical cancer research: an official journal of the American Association for Cancer Research. 2012; 18(20):5773-9. Epub 2012/08/17.
4. Filippakopoulos P, Qi J, Picaud S, Shen Y, Smith W B, Fedorov O, et al. Selective inhibition of BET bromodomains. Nature. 2010; 468(7327):1067-73. Epub 2010/09/28.
5. Matzuk M M, McKeown M R, Filippakopoulos P, Li Q, Ma L, Agno J E, et al. Small-molecule inhibition of BRDT for male contraception. Cell. 2012; 150(4):673-84. Epub 2012/08/21.
6. Grayson A R W E, Cameron M J, Godec J, Ashworth T, Ambrose J M, Aserlind A B, Wang H, Evan G, Kluk M J, Bradner J E, Aster J C, French C A. MYC, a downstream target of BRD-NUT, is necessary and sufficient for the blockade of differentiation in NUT midline carcinoma. Oncogene. 2013; in press(in press):in press.
7. Yang Z, Yik J H, Chen R, He N, Jang M K, Ozato K, et al. Recruitment of P-TEFb for stimulation of transcriptional elongation by the bromodomain protein Brd4. Molecular cell. 2005; 19(4):535-45.
8. Rahman S, Sowa M E, Ottinger M, Smith J A, Shi Y, Harper J W, et al. The Brd4 extraterminal domain confers transcription activation independent of pTEFb by recruiting multiple proteins, including NSD3. Molecular and cellular biology. 2011; 31(13):2641-52. Epub 2011/05/11.
9. Reynoird N, Schwartz B E, Delvecchio M, Sadoul K, Meyers D, Mukherjee C, et al. Oncogenesis by sequestration of CBP/p300 in transcriptionally inactive hyperacetylated chromatin domains. The EMBO journal. 2010; 29(17):2943-52. Epub 2010/08/03.
10. Haack H, Johnson L A, Fry C J, Crosby K, Polakiewicz R D, Stelow E B, et al. Diagnosis of NUT Midline Carcinoma Using a NUT-specific Monoclonal Antibody. The American journal of surgical pathology. 2009; 33(7):984-91.
11. Dang T P, Gazdar A F, Virmani A K, Sepetavec T, Hande K R, Minna J D, et al. Chromosome 19 translocation, overexpression of Notch3, and human lung cancer. Journal of the National Cancer Institute. 2000; 92(16):1355-7.
12. Haruki N, Kawaguchi K S, Eichenberger S, Massion P P, Gonzalez A, Gazdar A F, et al. Cloned fusion product from a rare t(15;19)(q13.2;p13.1) inhibit S phase in vitro. J Med Genet. 2005; 42(7):558-64.
13. Rosati R, La Starza R, Veronese A, Aventin A, Schwienbacher C, Vallespi T, et al. NUP98 is fused to the NSD3 gene in acute myeloid leukemia associated with t(8;11)(p11.2;p15). Blood. 2002; 99(10):3857-60. Epub 2002/05/03.
14. Wang G G, Cai L, Pasillas M P, Kamps M P. NUP98-NSD1 links H3K36 methylation to HoxA gene activation and leukaemogenesis. Nature cell biology. 2007; 9(7):804-12. Epub 2007/06/26.
15. Toretsky J A, Jenson J, Sun C C, Eskenazi A E, Campbell A, Hunger S P, et al. Translocation (11;15;19): a highly specific chromosome rearrangement associated with poorly differentiated thymic carcinoma in young patients. Am J Clin Oncol. 2003; 26(3):300-6.
16. Kees U R, Mulcahy M T, Willoughby M L. Intrathoracic carcinoma in an 11-year-old girl showing a translocation t(15;19). Am J Pediatr Hematol Oncol. 1991; 13(4):459-64.
17. Schwartz B E, Hofer M D, Lemieux M E, Bauer D E, Cameron M J, West N H, et al. Differentiation of NUT midline carcinoma by epigenomic reprogramming Cancer research. 2011; 71(7):2686-96. Epub 2011/03/31.
18. Thompson-Wicking K, Francis R W, Stirnweiss A, Ferrari E, Welch M D, Baker E, et al. Novel BRD4-NUT fusion isoforms increase the pathogenic complexity in NUT midline carcinoma. Oncogene. 2013; 32(39):4664-74. Epub 2012/11/07.
19. Engleson J, Soller M, Panagopoulos I, Dahlen A, Dictor M, Jerkeman M. Midline carcinoma with t(15;19) and BRD4-NUT fusion oncogene in a 30-year-old female with response to docetaxel and radiotherapy. BMC Cancer. 2006; 6:69.
20. Yan J, Diaz J, Jiao J, Wang R, You J. Perturbation of BRD4 protein function by BRD4-NUT protein abrogates cellular differentiation in NUT midline carcinoma. The Journal of biological chemistry. 2011; 286(31):27663-75. Epub 2011/06/10.
21. Muntean A G, Hess J L. The pathogenesis of mixed-lineage leukemia. Annual review of pathology. 2012; 7:283-301. Epub 2011/10/25.
22. Ball A, Bromley A, Glaze S, French C A, Ghatage P, Kobel M. A rare case of NUT midline carcinoma. Gynecologic oncology case reports. 2012; 3:1-3. Epub 2012/01/01.
23. Mills A F, Lanfranchi M, Wein R O, Mukand-Cerro I, Pilichowska M, Cowan J, et al. NUT Midline Carcinoma: A Case Report with a Novel Translocation and Review of the Literature. Head and neck pathology. 2013. Epub 2013/08/06.
24. Winkler K. Surgical treatment of pulmonary metastases in childhood. The Thoracic and cardiovascular surgeon. 1986; 34 Spec No 2:133-6. Epub 1986/11/01.
25. Li Y, Trojer P, Xu C F, Cheung P, Kuo A, Drury W J, 3rd, et al. The target of the NSD family of histone lysine methyltransferases depends on the nature of the substrate. The Journal of biological chemistry. 2009; 284(49):34283-95. Epub 2009/10/08.
26. Fang R, Barbera A J, Xu Y, Rutenberg M, Leonor T, Bi Q, et al. Human LSD2/KDM1b/AOF1 regulates gene transcription by modulating intragenic H3K4me2 methylation. Mol Cell. 2010; 39(2):222-33. Epub 2010/07/31.
27. Costanzi C, Pehrson J R. Histone macroH2A1 is concentrated in the inactive X chromosome of female mammals. Nature. 1998; 393(6685):599-601. Epub 1998/06/20.
28. Gamble M J, Frizzell K M, Yang C, Krishnakumar R, Kraus W L. The histone variant macroH2A1 marks repressed autosomal chromatin, but protects a subset of its target genes from silencing. Genes Dev. 2010; 24(1):21-32. Epub 2009/12/17.
29. Buschbeck M, Uribesalgo I, Wibowo I, Rue P, Martin D, Gutierrez A, et al. The histone variant macroH2A is an epigenetic regulator of key developmental genes. Nat Struct Mol Biol. 2009; 16(10):1074-9. Epub 2009/09/08.
30. Novikov L, Park J W, Chen H, Klerman H, Jalloh A S, Gamble M J. QKI-mediated alternative splicing of the histone variant MacroH2A1 regulates cancer cell proliferation. Molecular and cellular biology. 2011; 31(20):4244-55. Epub 2011/08/17.
31. Sporn J C, Jung B. Differential Regulation and Predictive Potential of MacroH2A1 Isoforms in Colon Cancer. Am J Pathol. 2012. Epub 2012/05/01.
32. Creppe C, Janich P, Cantarino N, Noguera M, Valero V, Musulen E, et al. MacroH2A1 regulates the balance between self-renewal and differentiation commitment in embryonic and adult stem cells. Molecular and cellular biology. 2012; 32(8):1442-52. Epub 2012/02/15.
33. Morishita M, di Luccio E. Cancers and the NSD family of histone lysine methyltransferases. Biochim Biophys Acta. 2011; 1816(2):158-63. Epub 2011/06/15.
34. Marango J, Shimoyama M, Nishio H, Meyer J A, Min D J, Sirulnik A, et al. The MMSET protein is a histone methyltransferase with characteristics of a transcriptional corepressor. Blood. 2008; 111(6):3145-54. Epub 2007/12/25.
35. Stec I, Wright T J, van Ommen G J, de Boer P A, van Haeringen A, Moorman A F, et al. WHSC1, a 90 kb SET domain-containing gene, expressed in early development and homologous to a Drosophila dysmorphy gene maps in the Wolf-Hirschhorn syndrome critical region and is fused to IgH in t(4;14) multiple myeloma. Human molecular genetics. 1998; 7(7):1071-82. Epub 1998/06/09.
36. Chesi M, Nardini E, Lim R S, Smith K D, Kuehl W M, Bergsagel P L. The t(4;14) translocation in myeloma dysregulates both FGFR3 and a novel gene, MMSET, resulting in IgH/MMSET hybrid transcripts. Blood. 1998; 92(9):3025-34. Epub 1998/10/27.
37. Garcia M J, Pole J C, Chin S F, Teschendorff A, Naderi A, Ozdag H, et al. A 1 Mb minimal amplicon at 8p11-12 in breast cancer identifies new candidate oncogenes. Oncogene. 2005; 24(33):5235-45. Epub 2005/05/18.
38. Angrand P O, Apiou F, Stewart A F, Dutrillaux B, Losson R, Chambon P. NSD3, a new SET domain-containing gene, maps to 8p12 and is amplified in human breast cancer cell lines. Genomics. 2001; 74(1):79-88. Epub 2001/05/26.
39. Yang Z Q, Liu G, Bollig-Fischer A, Giroux C N, Ethier S P. Transforming properties of 8p11-12 amplified genes in human breast cancer. Cancer research. 2010; 70(21):8487-97. Epub 2010/10/14.
40. Zhou Z, Thomsen R, Kahns S, Nielsen A L. The NSD3L histone methyltransferase regulates cell cycle and cell invasion in breast cancer cells. Biochemical and biophysical research communications. 2010; 398(3):565-70. Epub 2010/07/06.
41. Mann K M, Ward J M, Yew C C, Kovochich A, Dawson D W, Black M A, et al. Sleeping Beauty mutagenesis reveals cooperating mutations and pathways in pancreatic adenocarcinoma. Proceedings of the National Academy of Sciences of the United States of America. 2012; 109(16):5934-41. Epub 2012/03/17.
42. Mertz J A, Conery A R, Bryant B M, Sandy P, Balasubramanian S, Mele D A, et al. Targeting MYC dependence in cancer by inhibiting BET bromodomains. Proceedings of the National Academy of Sciences of the United States of America. 2011; 108(40):16669-74. Epub 2011/09/29.
43. Delmore J E, Issa G C, Lemieux M E, Rahl P B, Shi J, Jacobs H M, et al. BET bromodomain inhibition as a therapeutic strategy to target c-Myc. Cell. 2011; 146(6):904-17. Epub 2011/09/06.
44. Zuber J, Shi J, Wang E, Rappaport A R, Herrmann H, Sison E A, et al. RNAi screen identifies Brd4 as a therapeutic target in acute myeloid leukaemia. Nature. 2011; 478(7370):524-8. Epub 2011/08/05.
45. Bannister A J, Kouzarides T. Regulation of chromatin by histone modifications. Cell research. 2011; 21(3):381-95. Epub 2011/02/16.
46. Sharma S, Kelly T K, Jones P A. Epigenetics in cancer. Carcinogenesis. 2010; 31(1):27-36. Epub 2009/09/16.
47. Best J D, Carey N. Epigenetic opportunities and challenges in cancer. Drug discovery today. 2010; 15(1-2):65-70. Epub 2009/11/10.
48. Marino-Enriquez A, Hornick J L, Dal Cin P, Cibas E S, Qian X. Dedifferentiated liposarcoma and pleomorphic liposarcoma: a comparative study of cytomorphology and MDM2/CDK4 expression on fine-needle aspiration. Cancer cytopathology. 2014; 122(2):128-37. Epub 2013/11/15.
49. La Starza R, Barba G, Nofrini V, Pierini T, Pierini V, Marcomigni L, et al. Multiple EWSR1-WT1 and WT1-EWSR1 copies in two cases of desmoplastic round cell tumor. Cancer genetics. 2013; 206(11):387-92. Epub 2014/01/07.
50. Kim D, Salzberg S L. TopHat-Fusion: an algorithm for discovery of novel fusion transcripts. Genome biology. 2011; 12(8):R72. Epub 2011/08/13.
51. Trapnell C, Pachter L, Salzberg S L. TopHat: discovering splice junctions with RNA-Seq. Bioinformatics. 2009; 25(9):1105-11. Epub 2009/03/18.
52. Murphy G J, Mostoslaysky G, Kotton D N, Mulligan R C. Exogenous control of mammalian gene expression via modulation of translational termination. Nature medicine. 2006; 12(9):1093-9. Epub 2006/08/08.
53. Ince T A, Richardson A L, Bell G W, Saitoh M, Godar S, Karnoub A E, et al. Transformation of different human breast epithelial cell types leads to distinct tumor phenotypes. Cancer cell. 2007; 12(2):160-70.
54. Scheffner M, Munger K, Byrne J C, Howley P M. The state of the p53 and retinoblastoma genes in human cervical carcinoma cell lines. Proceedings of the National Academy of Sciences of the United States of America. 1991; 88(13):5523-7. Epub 1991/07/01.
55. Henikoff S, Ahmad K, Platero J S, van Steensel B. Heterochromatic deposition of centromeric histone H3-like proteins. Proceedings of the National Academy of Sciences of the United States of America. 2000; 97(2):716-21.
56. Senapedis W T, Kennedy C J, Boyle P M, Silver P A. Whole genome siRNA cell-based screen links mitochondria to Akt signaling network through uncoupling of electron transport chain. Molecular biology of the cell. 2011; 22(10):1791-805. Epub 2011/04/05.
57. Stelow E B, French C A. Carcinomas of the upper aerodigestive tract with rearrangement of the nuclear protein of the testis (NUT) gene (NUT midline carcinomas). Adv Anat Pathol. 2009; 16(2):92-6.

1. A method for treating cancer in a subject comprising administering to the subject an effective amount of a pharmaceutical composition comprising a NSD3 inhibitor.

2. The method of claim 1, wherein the cancer is a NSD3-dependent cancer.

3. The method of claim 2, wherein the NSD3-depdendent cancer is NUT midline carcinoma (NMC) characterized by the presence of at least one rearrangement of the NUT gene in a NMC tumor or cancer cell.

4. The method of claim 3, wherein the rearrangement in the NUT gene is a translocation of the NUT gene to form at least one of: a NSD3/NUT fusion gene, a BRD4/NUT fusion gene or a BRD3/NUT fusion gene.

5. The method of claim 1, wherein the cancer is selected from the group consisting of: primary breast carcinoma, pancreatic adenocarcinoma, acute myeloid leukemia or myelodysplastic syndrome with NUP98-NSD3 fusion oncogene.

6. The method of claim 4, wherein the cancer is selected from the group consisting of: leukemia, lymphoma, multiple myeloma, neuroblastoma, acute myeloid leukemia (AML), Burkitt lymphomia, Erythroleukemia, Lung adenocarcinoma, B-ALL (B-cell acute lymphoblastic leukemia), Burkitt Lymphoma, APML (Promyelocytic leukemia), Multiple myeloma, Cervical squamous cell carcinoma, Breast carcinoma, Prostate carcinoma or melanoma.

7. The method of claim 1, wherein the NSD3 inhibitor is selected from the group consisting of antibodies, antibody fragments, RNAi, siRNA, a NSD3 decoy molecule, or a polypeptide that blocks the binding of NSD3 with BRD4 and/or BRD3.

8. The method of claim 7, wherein the NSD3 decoy molecule comprises a portion of the ET domain of BRD4.

9. A method for treating NUT midline carcinoma (NMC) characterized by the rearrangement of the NUT gene to form a NSD3/NUT fusion gene, comprising administering to the subject an effective amount of a BET inhibitor.

10. The method of claim 9, wherein the BET inhibitor is a BRD4 inhibitor.

11. The method of claim 9, wherein the BET inhibitor is selected from the group consisting of: JQ1 ((S)-tert-butyl 2-(4-(4-chlorophenyl)-2,3,9-trimethyl-6H-thieno[3,2-f][1,2,4]triazolo[4,3-a][1,4]diazepin-6-yl)acetate), GSK-525762A, LY294002, 1-[2-(1/-/-benzimidazol-2-ylthio)ethyl]-1,3-dihydro-3-methyl-2H-benzinidazole-2-thione, 1-methylethyl ((2S,4R)-1-acetyl-2-methyl-6-{4-[(methylamino)methyl]phenyl}-1,2,3,4-tetrahydro-4-quinolinyl)carbamate, 2-[(4S)-6-(4-Chlorophenyl)-1-methyl-8-(methyloxy)-4H-[1,2,4]triazolo[4,3-a][1,4]benzodiazepin-4-yl]-N-ethylacetamide, 7-(3,5-dimethyl-4-isoxazolyl)-8-(methoxy)-1-[(1R)-1-(2-pyridinyl)ethyl]-1,3-dihydro-2H-imidazo[4,5-c]quinolin-2-one, 7-(3,5-dimethyl-4-isoxazolyl)-8-(methoxy)-1-[(1R)-phenylethyl]-2-(tetrahydro-2H-pyran-4-yl)-1H-imidazo[4,5-c]quinolone, 4-{(2S,4R)-1-acetyl-4-[(4-chlorophenyl)amino]-2-methyl-1,2,3,4-tetrahydro-6-quinolinyl}benzoic acid, and N-{1-methyl-7-[4-(1-piperidinylmethyl)phenyl][1,2,4]triazolo [4,3-a]quinolin-4-yl}urea.

12. The method of claim 9, comprising an initial step of detecting the presence of NSD3/NUT fusion oncoprotein or NSD3/NUT fusion gene prior to administering an effective amount of a BET inhibitor.

13.-15. (canceled)

16. A method of diagnosing and treating a subject with cancer, the method comprising:
(i) detecting whether a NSD3/NUT fusion oncoprotein or NSD3/NUT fusion gene is present in a biological sample obtained from the subject
(ii) diagnosing the subject with a NUT midline carcinoma (NMC) when the presence of a NSD3/NUT fusion oncoprotein or a NSD3/NUT fusion gene is detected; and
(iii) administering an effective amount of a NSD3 inhibitor or BET inhibitor to the diagnosed subject.

17. A method of diagnosing and treating a subject with cancer, the method comprising:
(i) detecting whether a rearrangement of the NUT gene is present in a biological sample obtained from the subject, wherein the rearrangement of NUT gene results in a BRD4/NUT or BRD3/NUT fusion protein or gene;
(ii) diagnosing the subject with a NUT midline carcinoma (NMC) when the presence of a BRD4/NUT or BRD3/NUT fusion protein or a BRD4/NUT or BRD3/NUT gene is detected; and
(iii) administering an effective amount of a NSD3 inhibitor to the diagnosed subject.

18. The method of claim 15, wherein the biological sample is a tissue sample or biopsy sample.

19. The method of claim 16, wherein the biological sample is a tissue sample or biopsy sample.


SEQUENCE LISTING (113 sequences)

SEQ ID NO: 1 - DNA, 5428 bp, Homo sapiens
gggggctttgtgcgcggcggcggcgggagaggcggcggcggcggccagcacggaggcggaggccgagggggctgtgcacaggtcgccgcggagaggcgtgcgaattccgagccgagcgccgaggaccgtgctacccaggccgggctgccagccgcaggctcctctctggcagcagcggcggcgcggcgacccccgtccctcggcctccccttcccatcccacctcccgagccttcctcttcccgcagcacgcccggcccggcccggccgtggccctcctcagtgccggccgccatggcagaggcgtccggcgcggggaaaatctagcccggggatttcatgcggcctagctcggttccgcctcctcctcgcgcggccccagcggctgcccgcaccccagccccactccgggcctccgtgtctctcctgtgatcgcactgacacggccggggggttagaatggaacaaactgaaggcccgatgagagaaagggaaagttaaggatgctggagcagaacaatggatttctctttctctttcatgcaagggatcatgggaaacacaattcagcaaccacctcaactcattgactccgccaacatccgtcaggaggatgcctttgataacaacagtgacattgctgaagatggtggccagacaccatatgaagctactttgcagcaaggctttcagtacccagctacaacagaagatcttcctccactcacaaatgggtatccatcatcaatcagtgtgtatgaaactcaaaccaaataccagtcatataatcagtatcctaatgggtcagccaatggctttggtgcagttagaaactttagccccactgactattatcattcagaaattccaaacacaagaccacatgaaattctggaaaaaccttcccctccacagccaccacctcctccttcggtaccacaaactgtgattccaaagaagactggctcacctgaaattaaactaaaaataaccaaaactatccagaatggcagggaattgtttgagtcttccctttgtggagaccttttaaatgaagtacaggcaagtgagcacacgaaatcaaagcatgaaagcagaaaagaaaagaggaaaaaaagcaacaagcatgactcatcaagatctgaagagcgcaagtcacacaaaatccccaaattagaaccagaggaacaaaatagaccaaatgagagggttgacactgtatcagaaaaaccaagggaagaaccagtactaaaagaggaagccccagttcagccaatactatcttctgttccaacaacggaagtgtccactggtgttaagtttcaggttggcgatcttgtgtggtccaaggtgggaacctatccttggtggccttgtatggtttcaagtgatccccagcttgaggttcatactaaaattaacacaagaggtgcccgagaatatcatgtccagttttttagcaaccagccagagagggcgtgggttcatgaaaaacgggtacgagagtataaaggtcataaacagtatgaagaattactggctgaggcaaccaaacaagccagcaatcactctgagaaacaaaagattcggaaaccccgacctcagagagaacgtgctcagtgggatattggcattgcccatgcagagaaagcattgaaaatgactcgagaagaaagaatagaacagtatacttttatttacattgataaacagcctgaagaggctttatcccaagcaaaaaagagtgttgcctccaaaaccgaagttaaaaaaacccgacgaccaagatctgtgctgaatactcagccagaacagaccaatgcaggggaggtggcctcctcactctcaagtactgaaattcggagacatagccagaggcggcacacaagtgcggaagaggaagagccaccgcctgttaaaatagcctggaaaactgcggcagcaaggaaatccttaccagcttccattacgatgcacaaagggagcctggatttgcagaagtgtaacatgtctccagttgtgaaaattgaacaagtgtttgctcttcagaatgctacaggggatgggaaatttatcgatcaatttgtttattcaacaaagggaattggtaacaaaacagaaataagtgtcagggggcaagacaggcttataatttctacaccaaaccagagaaatgaaaagccaacgcagagtgtatcatctcctgaagcaacatctggttctacaggctcagtagaaaagaagcaacagagaagatcaattagaactcgttctgaatcagagaaatccactgaggttgtgccaaagaagaagatcaaaaaggagcaggttgaaacagttcctcaggctacagtgaagactggattacagaaaggtgccagcgagatttcagattcctgtaaacctctaaagaaaaggagtcgcgcctcaactgatgtagaaatgactagttcagcatacagagacacatctgactccgattctagaggactgagtgacctgcaggtaggctttggaaagcaagtagatagcccttcagctactgcagatgcagacgtttctgatgtgcagtccatggattcaagtttgtcgagaagaggcactggaatgagtaagaaggacactgtatgtcagatttgtgaaagctctggtgactctctgattccttgtgagggagagtgctgcaaacactttcacctggagtgcctgggattggcatcacttcctgatagcaagttcatctgcatggaatgtaaaactgggcagcacccatgtttttcgtgtaaagtgtctggtaaagatgtgaagcgttgttctgttggtgcttgtgggaaattttatcatgaagcctgtgtccgcaaattccccactgccatctttgaatcaaaaggattccgctgtcctcagcactgctgctctgcctgctctatggagaaagatatccacaaagcaagtaaaggccgcatgatgagatgtttaagatgtccagttgcctatcactctggagatgcttgcattgcggccggaagcatgttagtatcctcctacattctcatctgtagtaatcattccaaacggagcagtaattcttctgctgtaaatgtaggcttttgtttcgtttgtgccagagggctgatagttcaggaccattcagaccccatgttcagttcatatgcctataagtcccactacctactgaatgaatcaaatcgtgctgagttgatgaaattacctatgattccttcttcgtcagcttccaaaaagaaatgtgagaaaggtggaagattgctctgctgtgaatcgtgcccagcttccttccacccggaatgcctaagcatagaaatgccagaaggctgctggaattgtaatgactgtaaagctggcaagaaactacattacaagcagattgtttgggtcaaattgggaaattacagatggtggccagcagagatctgcaaccccaggtctgtgccactgaacatccagggccttaaacatgacttgggggacttccctgtattcttctttggttctcatgactactactgggtacaccagggcagagtgttcccttatgttgaaggagacaaaagctttgctgaagggcagactagtattaacaagaccttcaaaaaggcactggaagaagctgcaaaacgtttccaggaattgaaagcacaaagagaaagtaaagaagccctagagattgaaaaaaactcaagaaaaccccctccctacaaacacatcaaagctaacaaagtaataggaaaggtgcagatccaggttgctgacctgtcagagattccccgctgtaactgcaagccagctgatgaaaacccttgtggcttggaatcggagtgcctgaacagaatgttgcagtatgaatgccacccgcaggtgtgcccagctggagatcgttgtcagaaccagtgctttacaaagagactataccctgatgcagagatcatcaaaacggagcggagaggctggggcctcaggaccaaaaggagcattaagaagggtgaatttgtaaatgaatacgtcggtgaattaattgatgaagaagaatgcagattgcgaatcaagcgagcccacgagaacagtgtaactaatttttatatgttaactgttaccaaggaccgtataattgatgccggcccaaaaggaaattattctcgcttcatgaaccacagttgtaatcccaactgtgaaacacaaaagtggacagtgaatggagatgttcgagtgggactatttgctctctgtgatattcctgcagggatggagttaacatttaattataacctagattgtctgggcaacggcagaacggagtgccactgtggagcagataactgcagtggttttctaggagtgcggccaaagtcggcatgtgcgtcaacaaatgaagagaaggcaaaaaatgctaagttaaaacagaagagacgaaagatcaaaacagaaccaaagcagatgcatgaagattactgttttcaatgtggagatggtggagagctggtcatgtgtgacaaaaaagactgtcccaaagcataccacctcctatgccttaacctgactcagccaccatatggaaagtgggagtgtccgtggcatcagtgcgatgagtgcagcagtgcagctgtttccttctgtgaattctgtccacattcattttgtaaagatcatgaaaagggggccctggttccctctgcactggaaggccgcctctgctgctcggaacatgaccccatggctcctgtgtcaccagaatactggagcaagataaaatgtaaatgggaatcacaagatcatggagaagaagtaaaagaataaatgtgtggtgtcccctcctttctatttaagtgaaaaaagcaaatagatcatgcatttaaaaagaagagactgctacagtgcatacagcctttgccatcggaactgccttattaaagcaaaaatgggaaaccagttcatgcaggcagaagcagttggtggtgtctggtttttgtttgatttggttggtttgggattcttttgtggagggttaaattcccttggtcttttcttgccttttattgtgcttcagtgccattgcagcttgaaaaagaaatgtttttgctgttaaaataagaacaaagagaaaagtaagttttgttaatgagataaatttaaagtctaagatgtgttccttggttgtataaagcaaaagtagccatcattcctttatttattttcatttttaggaatttcaagaagtgtagttcaatagtctaatcaagtgtgtgtgtgttttaagtaggaatctgagaaagccctctaggaaagggtatgataagctttatatacctctttactgagcagtaggtaggctcacttctctttcccttcaaaatgcttttcatagtagagaagggctctatggaagtattaaa

SEQ ID NO: 2 - protein, 1437 aa, Homo sapiens
MDFSFSFMQGIMGNTIQQPPQLIDSANIRQEDAFDNNSDIAEDGGQTPYEATLQQGFQYPATTEDLPPLTNGYPSSISVYETQTKYQSYNQYPNGSANGFGAVRNFSPTDYYHSEIPNTRPHEILEKPSPPQPPPPPSVPQTVIPKKTGSPEIKLKITKTIQNGRELFESSLCGDLLNEVQASEHTKSKHESRKEKRKKSNKHDSSRSEERKSHKIPKLEPEEQNRPNERVDTVSEKPREEPVLKEEAPVQPILSSVPTTEVSTGVKFQVGDLVWSKVGTYPWWPCMVSSDPQLEVHTKINTRGAREYHVQFFSNQPERAWVHEKRVREYKGHKQYEELLAEATKQASNHSEKQKIRKPRPQRERAQWDIGIAHAEKALKMTREERIEQYTFIYIDKQPEEALSQAKKSVASKTEVKKTRRPRSVLNTQPEQTNAGEVASSLSSTEIRRHSQRRHTSAEEEEPPPVKIAWKTAAARKSLPASITMHKGSLDLQKCNMSPVVKIEQVFALQNATGDGKFIDQFVYSTKGIGNKTEISVRGQDRLIISTPNQRNEKPTQSVSSPEATSGSTGSVEKKQQRRSIRTRSESEKSTEVVPKKKIKKEQVETVPQATVKTGLQKGASEISDSCKPLKKRSRASTDVEMTSSAYRDTSDSDSRGLSDLQVGFGKQVDSPSATADADVSDVQSMDSSLSRRGTGMSKKDTVCQICESSGDSLIPCEGECCKHFHLECLGLASLPDSKFICMECKTGQHPCFSCKVSGKDVKRCSVGACGKFYHEACVRKFPTAIFESKGFRCPQHCCSACSMEKDIHKASKGRMMRCLRCPVAYHSGDACIAAGSMLVSSYILICSNHSKRSSNSSAVNVGFCFVCARGLIVQDHSDPMFSSYAYKSHYLLNESNRAELMKLPMIPSSSASKKKCEKGGRLLCCESCPASFHPECLSIEMPEGCWNCNDCKAGKKLHYKQIVWVKLGNYRWWPAEICNPRSVPLNIQGLKHDLGDFPVFFFGSHDYYWVHQGRVFPYVEGDKSFAEGQTSINKTFKKALEEAAKRFQELKAQRESKEALEIEKNSRKPPPYKHIKANKVIGKVQIQVADLSEIPRCNCKPADENPCGLESECLNRMLQYECHPQVCPAGDRCQNQCFTKRLYPDAEIIKTERRGWGLRTKRSIKKGEFVNEYVGELIDEEECRLRIKRAHENSVTNFYMLTVTKDRIIDAGPKGNYSRFMNHSCNPNCETQKWTVNGDVRVGLFALCDIPAGMELTFNYNLDCLGNGRTECHCGADNCSGFLGVRPKSACASTNEEKAKNAKLKQKRRKIKTEPKQMHEDYCFQCGDGGELVMCDKKDCPKAYHLLCLNLTQPPYGKWECPWHQCDECSSAAVSFCEFCPHSFCKDHEKGALVPSALEGRLCCSEHDPMAPVSPEYWSKIKCKWESQDHGEEVKE

SEQ ID NO: 3 - DNA, 1707 bp, Homo sapiens
atggatttctctttctctttcatgcaagggatcatgggaaacacaattcagcaaccacctcaactcattgactccgccaacatccgtcaggaggatgcctttgataacaacagtgacattgctgaagatggtggccagacaccatatgaagctactttgcagcaaggctttcagtacccagctacaacagaagatcttcctccactcacaaatgggtatccatcatcaatcagtgtgtatgaaactcaaaccaaataccagtcatataatcagtatcctaatgggtcagccaatggctttggtgcagttagaaactttagccccactgactattatcattcagaaattccaaacacaagaccacatgaaattctggaaaaaccttcccctccacagccaccacctcctccttcggtaccacaaactgtgattccaaagaagactggctcacctgaaattaaactaaaaataaccaaaactatccagaatggcagggaattgtttgagtcttccctttgtggagaccttttaaatgaagtacaggcaagtgagcacacgaaatcaaagcatgaaagcagaaaagaaaagaggaaaaaaagcaacaagcatgactcatcaagatctgaagagcgcaagtcacacaaaatccccaaattagaaccagaggaacaaaatagaccaaatgagagggttgacactgtatcagaaaaaccaagggaagaaccagtactaaaagaggaagccccagttcagccaatactatcttctgttccaacaacggaagtgtccactggtgttaagtttcaggttggcgatcttgtgtggtccaaggtgggaacctatccttggtggccttgtatggtttcaagtgatccccagcttgaggttcatactaaaattaacacaagaggtgcccgagaatatcatgtccagttttttagcaaccagccagagagggcgtgggttcatgaaaaacgggtacgagagtataaaggtcataaacagtatgaagaattactggctgaggcaaccaaacaagccagcaatcactctgagaaacaaaagattcggaaaccccgacctcagagagaacgtgctcagtgggatattggcattgcccatgcagagaaagcattgaaaatgactcgagaagaaagaatagaacagtatacttttatttacattgataaacagcctgaagaggctttatcccaagcaaaaaagagtgttgcctccaaaaccgaagttaaaaaaacccgacgaccaagatctgtgctgaatactcagccagaacagaccaatgcaggggaggtggcctcctcactctcaagtactgaaattcggagacatagccagaggcggcacacaagtgcggaagaggaagagccaccgcctgttaaaatagcctggaaaactgcggcagcaaggaaatccttaccagcttccattacgatgcacaaagggagcctggatttgcagaagtgtaacatgtctccagttgtgaaaattgaacaagtgtttgctcttcagaatgctacaggggatgggaaatttatcgatcaatttgtttattcaacaaagggaattggtaacaaaacagaaataagtgtcagggggcaagacaggcttataatttctacaccaaaccagagaaatgaaaagccaacgcagagtgtatcatctcctgaagcaacatctggttctaca

SEQ ID NO: 4 - protein, 569 aa, Homo sapiens
MDFSFSFMQGIMGNTIQQPPQLIDSANIRQEDAFDNNSDIAEDGGQTPYEATLQQGFQYPATTEDLPPLTNGYPSSISVYETQTKYQSYNQYPNGSANGFGAVRNFSPTDYYHSEIPNTRPHEILEKPSPPQPPPPPSVPQTVIPKKTGSPEIKLKITKTIQNGRELFESSLCGDLLNEVQASEHTKSKHESRKEKRKKSNKHDSSRSEERKSHKIPKLEPEEQNRPNERVDTVSEKPREEPVLKEEAPVQPILSSVPTTEVSTGVKFQVGDLVWSKVGTYPWWPCMVSSDPQLEVHTKINTRGAREYHVQFFSNQPERAWVHEKRVREYKGHKQYEELLAEATKQASNHSEKQKIRKPRPQRERAQWDIGIAHAEKALKMTREERIEQYTFIYIDKQPEEALSQAKKSVASKTEVKKTRRPRSVLNTQPEQTNAGEVASSLSSTEIRRHSQRRHTSAEEEEPPPVKIAWKTAAARKSLPASITMHKGSLDLQKCNMSPVVKIEQVFALQNATGDGKFIDQFVYSTKGIGNKTEISVRGQDRLIISTPNQRNEKPTQSVSSPEATSGST

SEQ ID NO: 5 - DNA, 249 bp, Homo sapiens
tcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagct

SEQ ID NO: 6 - protein, 82 aa, Homo sapiens
SEEEDKCKPMSYEEKRQLSLDINKLPGEKLGRVVHIIQSREPSLKNSNPDEIEIDFETLKPSTLRELERYVTSCLRKKRKPQ

SEQ ID NO: 7 - DNA, 3795 bp, Homo sapiens
gagttccgtattctagttctgtgtgatctgatctttaccttcccttccttggatccctgtgcacctactggagccaggttactctgggtcctggacctgactgcctcattctggaggcttccagacagccacagttagtgcccaaacctgagaggatggcttcagatggagcatctgcattgccgggaccggatatgagcatgaaacctagtgccgccctgtctccatcccctgcacttccctttctcccaccaacttctgacccaccagaccacccacccagggagccacctccacagcccatcatgccttcagtattctctccagacaaccctctgatgctctctgctttccccagctcactgttggtgacaggggacgggggcccttgcctcagtggggctggggctggcaaggtcattgtcaaagtcaagacagaaggggggtcagctgagccctctcaaactcagaactttatccttactcagactgccctcaattcgactgccccgggcactccctgtggaggccttgagggtcctgcacctccatttgtgacagcatctaatgtgaagaccattctgccctctaaggctgttggtgtcagccaggagggtcctccaggccttccgcctcagcctccaccaccagttgctcaactggtccccattgtgcccctggaaaaagcttggccagggccacatgggacaaccggggaaggaggtcctgtggccactctatccaagccttccctaggtgaccgctccaaaatttccaaggacgtttatgagaacttccgtcagtggcagcgttacaaagccttggcccggaggcacctatcccagagtcctgacacagaagctctttcctgttttcttatcccagtgcttcgttccctggcccggctgaagcccactatgaccctggaggagggactgccattggctgtgcaggagtgggagcacaccagcaactttgaccggatgatcttttatgagatggcagaaaggttcatggagtttgaggctgaggagatgcagattcagaacacacagctgatgaatgggtctcagggcctgtctcctgcaacccctttgaaacttgatcctctagggcccctggcctctgaggtttgccagcagccagtgtacattccgaagaaggcagcctccaagacacgggccccccgccggcgtcagcgtaaagcccagagacctcctgctcctgaggcacccaaggagatcccaccagaagctgtgaaggagtatgttgacatcatggaatggctggtggggactcacttggccactggggagtcagatggaaaacaagaggaagaagggcagcagcaggaggaggaagggatgtatccagatccaggtctcctgagctacatcaatgagctgtgttctcagaaggtctttgtctccaaggtggaggctgtcattcaccctcaatttctggcagatctgctgtccccagaaaaacagagagatcccttggccttaattgaggagctagagcaagaagaaggactcactcttgcccagctggtccagaagcgactcatggccttggaagaggaggaagatgcagaggcgcctccaagtttcagtggcgctcagttggactcaagtccttctggttctgttgaggatgaagatggggatgggcggcttcggccctcacctgggcttcagggggctgggggcgccgcttgccttggaaaggtttcttcttcaggaaaacgggcaagagaagtgcatggtgggcaggagcaagccctagatagccccagagggatgcacagggatgggaacactctgccatcccccagcagctgggacctgcagccagaacttgcagctccacagggaactccgggacccttgggtgtggagaggagagggtctgggaaggttataaaccaggtatctctacatcaggatggccatctaggaggcgctgggcctcctgggcactgcctggtggctgataggacttcagaggctctgcccctttgttggcagggaggcttccagcctgagagcactcccagtttggatgctggacttgcagagctggctcctctgcaaggacaagggttagaaaagcaagtcctgggattgcagaaaggacaacaaacagggggtcgtggagtgcttcctcaagggaaggagcctttagcagtgccctgggaaggctcttcaggagccatgtggggagatgacagaggtacccccatggctcagagttatgatcagaatccttcccctagagcagctggggagagggacgatgtctgtctcagcccaggagtttggctgagcagtgagatggatgctgtaggcttggagctgcctgtacaaatagaggaggtcatagagagcttccaagttgagaagtgtgtaactgagtatcaggaaggctgccagggactgggctccaggggcaacatttccctgggtcctggagaaaccctagtacctggggatacggagagcagtgtgattccctgtggaggcacagttgcggcagctgccctagaaaagagaaactattgcagcttgccaggacctttgagggccaacagcccacccttgaggtccaaagaaaatcaagaacagagctgtgaaaccgtagggcatcccagtgatctgtgggcagaaggttgcttcccattgctagaaagtggtgattccacactggggtcttccaaagaaacccttccacccacatgccaaggcaatctccttatcatggggactgaggatgcctcctccttgcctgaagccagtcaagaggcagggagcagaggcaattccttttctcctctgttggaaaccatagaacctgtcaacatactagatgttaaagatgactgtggcctccaactaagggtcagcgaggacacctgcccactgaatgttcattcttatgacccccaaggagaaggcagggtggatcctgatctgtccaagcctaaaaaccttgctcctttacaagagagtcaggagtcttacacaactgggactcccaaagcaacatcttctcaccagggccttggaagcactttgcctagaaggggaaccaggaatgccatagttccgagagaaacttctgttagtaaaacacacaggtcagcagacagggccaaaggaaaggagaaaaagaaaaaggaagcagaggaagaggatgaggaactctccaactttgcttacctcttggcctctaaacttagcctctcaccaagggagcatcccctcagtcctcaccatgcctcaggaggtcagggcagccagagagcatcccacctgctccctgctggagcaaaaggccccagcaaacttccatatcctgttgccaagtctgggaagcgagctctagctggaggtccagcccctactgaaaagacaccccactcaggagctcaacttggggtccccagggagaaacccctagctctgggagtagttcgaccctcacagcctcgtaaaaggcggtgtgacagttttgtcacgggcagaaggaagaaacgacgtcgtagccagtagggagcagcgggaccatctgaccccacttgccagtccctaaaggtgggtgccccagagtagattccacccctgctgcccaccaatggagaatcccaatgttgaatctcatcccaatgttgttttgttgttctgcaaaagtggcaagcatggagagagaggtcagactggctaggctgcagggggaattacctttggaaggagctatatagaaaaaaaatgaataaagtgttttgttggaaaa

SEQ ID NO: 8 - protein, 1131 aa, Homo sapiens
MASDGASALPGPDMSMKPSAALSPSPALPFLPPTSDPPDHPPREPPPQPIMPSVFSPDNPLMLSAFPSSLLVTGDGGPCLSGAGAGKVIVRVKTEGGSAEPSQTQNFILTQTALNSTAPGTPCGGLEGPAPPFVTASNVKTILPSKAVGVSQEGPPGLPPQPPPPVAQLVPIVPLEKAWPGPHGTTGEGGPVATLSKPSLGDRSKISKDVYENFRQWQRYKALARRHLSQSPDTEALSCFLIPVLRSLARLKPTMTLEEGLPLAVQEWEHTSNFDRMIFYEMAERFMEFEAEEMQIQNTQLMNGSQGLSPATPLKLDPLGPLASEVCQQPVYIPKKAASKTRAPRRRQRKAQRPPAPEAPKEIPPEAVKEYVDIMEWLVGTHLATGESDGKQEEEGQQQEEEGMYPDPGLLSYINELCSQKVFVSKVEAVIHPQFLADLLSPEKQRDPLALIEELEQEEGLTLAQLVQKRLMALEEEEDAEAPPSFSGAQLDSSPSGSVEDEDGDGRLRPSPGLQGAGGAACLGKVSSSGKRAREVHGGQEQALDSPRGMHRDGNTLPSPSSWDLQPELAAPQGTPGPLGVERRGSGKVINQVSLHQDGHLGGAGPPGHCLVADRTSEALPLCWQGGFQPESTPSLDAGLAELAPLQGQGLEKQVLGLQKGQQTGGRGVLPQGKEPLAVPWEGSSGAMWGDDRGTPMAQSYDQNPSPRAAGERDDVCLSPGVWLSSEMDAVGLELPVQIEEVIESFQVEKCVTEYQEGCQGLGSRGMISLGPGETLVPGDTESSVIPCGGTVAAAALEKRNYCSLPGPLRANSPPLRSKENQEQSCETVGHPSDLWAEGCFPLLESGDSTLGSSKETLPPTCQGNLLIMGTEDASSLPEASQEAGSRGSFSPLLETIEPVNILDVKDDCGLQLRVSEDTCPLNVHSYDPQGEGRVDPDLSKPKNLAPLQESQESYTTGTPKATSSHQGLGSTLPRRGTRNAIVPRETSVSKTHRSADRAKGKEKKKKEAEEEDEELSNFAYLLASKLSLSPREHPLSPHHASGGQGSQRASHLLPAGAKGPSKLPYPVAKSGKRALAGGPAPTEKTPHSGAQLGVPREKPLALGVVRPSQPRKRRCDSFVTGRRKKRRRSQ

SEQ ID NO: 9 - DNA, 5198 bp, Homo sapiens
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaaggaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctgagcgcacctaggtggcttctgactttgattttctggcaaaacattgactttccatagtgttaggggcggtggtggaggtgggatcagcggccaggggatgcctcagggcctggccctcctgcatgctatgcccggggcaggcctgacgggcagctgaggattgcagagcctgtctgccttacggccagtcggacagacgtcccgccacccaccacccctcacaggacgtccgctcagcacacgccttgttacgagcaagtgccggctggacccaagccctgcatccccacatgcggggcagaggcccttctctccgccaaatgtctacacagtatacacaggacatcgttgctgccgccgtgactggttttctgtccccaagaacgtgacgttcgtgatgtcctgcccgccgggagtctttccccacaccccagccatcgccgcccgctcccaggaggccagggcaggcctgcgtgggctggaggcgggcgaggccggcccaccccctcgctggcactgactttgccttgaacagaccccccgaccctcccccacaagcctttaattgagagccgctctctgtaagtgtttgcttgtgcaaaagggaatagtgccgtggaggtgtgtgtgtccatggcatccggagcgaggcgactgtcctgcgtgggtagccctcggccggggagtgaggccaccaaccaaagtcagttccttcccacctgtgtttctgtttcgtttttttttttcttttttttctatatatattttttgttgaattctattttatttttaattctctcttctcctccagacacaatggcactgcttatctccgaaatggtgtgatcgtctcctcattgagcagcggctgccaccgcgctgtgggta

SEQ ID NO: 10 - protein, 1359 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQRINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTRKGVKRKADTTTPTTIDPIHEPPSLPPEPKTTKLGQRRESSRPVKPPKKDVPDSQQHPAPERSSKVSEQLKCCSGILKEMFAKKHAAYAWPFYKPVDVEALGLHDYCDIIKHPMDMSTIKSKLEAREYRDAQEFGADVRLMFSNCYKYPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSRKEPAPMKSKPPPTYESEEEDKCKPMSYEEKRQLSLDINLPGEKLGRVVHIIQSREPSLKNSNPDEIEIDFETLKPSTLRELERYVTSCLRKKRKPQAEICVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLWVKEEKIHSPIIRSEPFSPSLRPEPPKHPESIAPVHLPQRPEMKPVDVGRPVIRPPEQNAPPPGAPDKDKQKQEPKTPVAPKKDLKIKNMGSWASLVQKHPTTPSSTARSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 11 - DNA, 5673 bp, Homo sapiens
tgccggggccggcgagccaaagaggagccggccgcgcgggccgggaggggacggccgccggagccgcgaggccaactgtcgcctggttgggcccggaaatgggacgtcgcgctttctcagggagcgtagaagcagccagggcctctccaagccgctgctgtgacagaaagtgagtgagctgccggaggatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctacgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccccgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagacagctgaagaaaggcggcaagcaggcatctgcctcctacgactcagaggaagaggaggagggcctgcccatgagctacgatgaaaagcgccagcttagcctggacatcaaccggctgcccggggagaagctgggccgggtagtgcacatcatccaatctcgggagccctcgctcagggactccaaccccgacgagatagaaattgactttgagactctgaaacccaccactttgcgggaactggagagatatgtcaagtcttgtttacagaaaaagcaaaggaaaccgttctcagcaagcgggaagaaacaggcagccaagtcgaaagaggagctagctcaggaaaagaagaaggagctggaaaagcgtctgcaggatgtcagcgggcagctgagcagcagcaagaagcccgcccggaaagagaagcccggctcagcaccctcagggggcccgtccaggctcagcagcagcagctcctccgagtctgggagcagcagctccagcgggtccagctctgacagcagtgactcagaatgaactggcttcggacagaacaggacagatggatgtcgcacacgccgagactctgccgtacccctctgtggttcatattactacttctgttccatggtgtgcaggtctgcttcttaattcagtgttatgatatcttccagtttttgctttcataggtcagagatctatcttgtgtgtgcgttagacttgatgagaaggtgtgaactctgcagaaagtctcttcttcatcactgaattcagtcacttggagatgacaacttcaaatgctaacccgatgaccccagaaaaccgtgtgagattcgtaccgaagaaccttgtggaatccctttgcttaggcccaacctggtcgatagctcgagaaagaattttttccaaggaaatgtctcggatatgggtactgtatttgaaagctgttagctttgtcaacacgcattgtccttgtcatttgggccccgagctctgaccctcgtgtctgacgcggccacctctttctggaggggctgaggacagatgtgcctgcttgtggagaccaggctgggcctaagcgaagggtcatcgcagccccagcccggagcgtggagcccttggggggtggtcgggtgggatgtgcgttctccgctcgtggtgatgtcaggagctcctcggagggaacagagcggctgtgtatgcagcctgcaggtttccatacactgaagcttttacctcaactttaaaaaaaaaaaaagaagaaaagatttaaaaaaaaaaaaaaaggagactttttttgtaaggttcggcaattttctacggaagtccagcccgctgtgagtccccgcctggctagcgctgcccctgctgctgctgccgcgccacccttgggaaccacttcccgagcttatgtgtatataaatagttatttattaacatcattggtcatttttaaaaaaaagaaaataagaaaaaaccgcagaagaaatgcattcacacagtcgcagagatgcaggccttgccagtggtgtgccgggcgcgggtcctgtctggcggcggcctgtcgtctccaggtgctaactcctgccaccgcgcggtgctcacccacgtctgttcgcgcgctcgcccctgggtttgttgggttttttggtttttttctttgtggtttttttttttttttttttttgtatgaaacttggaggcttacaggtatagacagctttcagctacagcacattctaattttttattttgtttagttcttttgtattcacttctggtctctttaagactgttttaaaagaaatcaatttagggaaccccagttatataatataaactttgtaatctgagagaaaaaatgtatagtaaatctaagtcttgatttttaactttctattgtaaaaaataataatatacagagtttaatagaaggtgatgttttggttttgttttcccagaggctgccatatggtctttgagtacggggatgtcccaaactggcccaccaatgagcatggcggctccggccaggaatgccagagttagcctcccaggcttgcgggtggacatgcctgctccctgccagcctccagtggcctggccaggccctcccgagcctgtctgccctccccaggggtggaggagtctctgggccccaggaggattccctcccggagactcgcacggtgctccctgctcacgcgttgtcacagttagtccggaaatgactgaaaccaggcattctcccggacctcagcgtgggggagcctccaggcagacgctgggtatggagctgtggtgtggtctgtcctgtatggtggccagtgctttctgccagcatttctggatggatatagggactatcattagtatcctaatacacggtgattttaaaacaaccataaaattgattcagagtccactgacccttacagatgtaggtatacccttactggagagggaactctgatgaggagatgctggtaaattatcattttttaaattgctggtgagtctgacacttggtgagttttcagccagtttgttaaacttttaattaagttttgtttataataaaaatataaatggatttgaaagtttccattttttaaagttaccctcgttttcaaaggtattttctaaacagatctttaatggactatttaaaccgaatttaaggaattcacacacgacagttgacaggtcttcacgcaggctggttggtaacgtgctgccagcacagggctgggtgatacgtacaccctaagccgggggtgcctggggctggggggcgctccttgcaatgcccctccagccacagggcagtgaggtgctgcctgtgtgagccgtcgggggagcggccggctgtgggggcagcgcagcaggagcatcgtggggcctttccttctcggctggttctctgtgacggtggcgtcggctcgcctctgctcctttcatctagaaagaagccactgaccctgacagcccacggcgggtacactgagcagctgcattggtgctgtcacttttttaaggctttctgtccagacttcaacactggtttcttttcagagtttcgaaggattaatgacttcctcagcgcccttgctggcgggctgagggtgacagtcacgtccgtttcttctgtattagaaggctgcggtgattcaattagattgtcccactgctgagacctgtagggcagcttctaacatgcttttttcaaggggagaggagtagtgacaagtcgtgtgtcggaattggatttgagaacactctgaatgacccctggaggccgagggggcaggcttcgggcgtgaactgaactccagacccctctttgtgttgggcagtgtcatcttgcttacaaactgtaagacacatttttttgtgtgtttgtttttgttgttgttcttttgcagcactcacgcctctgacagtcttttgggaaagagtaacacccacatacagaatttgtcacatccagagtagcactgttccttaatactggcataatgcttccaggaagtttttcttttttatatttaaaatgttacttttctgtatgatgtgcatgcaagtttaccgtaacttttcttaaactttttagtgccgtttctagtatattcctgtaaatgtcagttactgaaaatgagtccaatgtaagtagtttagcttgtttattgcaatgctggcctcaacacaacagaataaaaatggtagaaagtactctttgatgtttctggtaatcatggacccttctcctggggcatttgttttgttttcataataaaaagcaaaaaaaaaaaaaaaaaaaaaaaaaaaaaaaaaaaaaaaa

SEQ ID NO: 12 - protein, 725 aa, Homo sapiens
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRQLKKGGKQASASYDSEEEEEGLPMSYDEKRQLSLDINRLPGEKLGRVVHIIQSREPSLRDSNPDEIEIDFETLKPTTLRELERYVKSCLQKKQRKPFSASGKKQAAKSKEELAQEKKKELEKRLQDVSGQLSSSKKPARKEKPGSAPSGGPSRLSSSSSSESGSSSSSGSSSDSSDSE

SEQ ID NO: 13 - protein, 4 aa, Homo sapiens
PWWP

SEQ ID NO: 14 - DNA, 20 bp, Artificial Sequence
  Description of Artificial Sequence Synthetic primer
aagagccaccgcctgttaaa

SEQ ID NO: 15 - RNA, 21 nt, Artificial Sequence
  Description of Artificial Sequence Synthetic oligonucleotide
cuguaaaccucuaaagaaauu

SEQ ID NO: 16 - RNA, 21 nt, Artificial Sequence
  Description of Artificial Sequence Synthetic oligonucleotide
gaaaggugccagcgagauuuu

SEQ ID NO: 17 - RNA, 19 nt, Artificial Sequence
  Description of Artificial Sequence Synthetic oligonucleotide
gaacgugcucagugggaua

SEQ ID NO: 18 - RNA, 19 nt, Artificial Sequence
  Description of Artificial Sequence Synthetic oligonucleotide
gcuugagguucauacuaaa

SEQ ID NO: 19 - protein, 29 aa, Unknown
  Description of Unknown GALA peptide
AALEALAEALEALAEALEALAEAAAAGGC

SEQ ID NO: 20 - protein, 30 aa, Unknown
  Description of Unknown EALA peptode
AALAEALAEALAEALAEALAEALAAAAGGC

SEQ ID NO: 21 - protein, 15 aa, Artificial Sequence
  Description of Artificial Sequence Synthetic peptide
ALEALAEALEALAEA

SEQ ID NO: 22 - protein, 22 aa, Unknown
  Description of Unknown INF-7 peptide
GLFEAIEGFIENGWEGMIWDYG

SEQ ID NO: 23 - protein, 23 aa, Unknown
  Description of Unknown Inf HA-2 peptide
GLFGAIAGFIENGWEGMIDGWYG

SEQ ID NO: 24 - protein, 48 aa, Unknown
  Description of Unknown diINF-7 peptide
GLFEAIEGFIENGWEGMIDGWYGCGLFEAIEGFIENGWEGMIDGWYGC

SEQ ID NO: 25 - protein, 44 aa, Unknown
  Description of Unknown diINF-3 peptide
GLFEAIEGFIENGWEGMIDGGCGLFEAIEGFIENGWEGMIDGGC

SEQ ID NO: 26 - protein, 35 aa, Unknown
  Description of Unknown GLF peptide
GLFGALAEALAEALAEHLAEALAEALEALAAGGSC

SEQ ID NO: 27 - protein, 34 aa, Unknown
  Description of Unknown GALA-INF3 peptide
GLFEAIEGFIENGWEGLAEALAEALEALAAGGSC

SEQ ID NO: 28 - protein, 41 aa, Unknown
  Description of Unknown INF-5 peptide
GLFEAIEGFIENGWEGLIDGKGLFEAIEGFIENGWEGLIDG

SEQ ID NO: 29 - protein, 19 aa, Unknown
  Description of Unknown JTS-1 peptide
LFEALLELLESLWELLLEA

SEQ ID NO: 30 - protein, 20 aa, Unknown
  Description of Unknown ppTG1 peptide
GLFKALLKLLKSLWKLLLKA

SEQ ID NO: 31 - protein, 20 aa, Unknown
  Description of Unknown ppTG20 peptide
GLFRALLRLLRSLWRLLLRA

SEQ ID NO: 32 - protein, 30 aa, Unknown
  Description of Unknown KALA peptide
WEAKLAKALAKALAKHLAKALAKALKACEA

SEQ ID NO: 33 - protein, 22 aa, Unknown
  Description of Unknown HA peptide
GLFFEAIAEFIEGGWEGLIEGC

SEQ ID NO: 34 - protein, 26 aa, Unknown
  Description of Unknown Melittin peptide
GIGAVLKVLTTGLPALISWIKRKRQQ

SEQ ID NO: 35 - protein, 8 aa, Artificial Sequence
  Description of Artificial Sequence Synthetic peptide
HHHHHWYG

SEQ ID NO: 36 - protein, 10 aa, Artificial Sequence
  Description of Artificial Sequence Synthetic peptide
CHKKKKKKHC

SEQ ID NO: 37 - protein, 16 aa, Unknown
  Description of Unknown penetratin peptide
RQIKIWFQNRRMKWKK

SEQ ID NO: 38 - protein, 14 aa, Human immunodeficiency virus
GRKKRRQRRRPPQC

SEQ ID NO: 39 - protein, 27 aa, Unknown
  Description of Unknown signal sequence based peptide
GALFLGWLGAAGSTMGAWSQPKKKRKV

SEQ ID NO: 40 - protein, 18 aa, Unknown
  Description of Unknown PVEC peptide
LLIILRRRIRKQAHAHSK

SEQ ID NO: 41 - protein, 26 aa, Unknown
  Description of Unknown transportan peptide
GWTLNSAGYLLKINLKALAALAKKIL

SEQ ID NO: 42 - protein, 18 aa, Unknown
  Description of Unknown amphiphilic model peptide
KLALKLALKALKAALKLA

SEQ ID NO: 43 - protein, 9 aa, Artificial Sequence
  Description of Artificial Sequence Synthetic peptide
RRRRRRRRR

SEQ ID NO: 44 - protein, 10 aa, Unknown
  Description of Unknown bacterial cell wall permeating peptide
KFFKFFKFFK

SEQ ID NO: 45 - protein, 37 aa, Unknown
  Description of Unknown LL-37 peptide
LLGDFFRKSKEKIGKEFKRIVQRIKDFLRNLVPRTES

SEQ ID NO: 46 - protein, 31 aa, Unknown
  Description of Unknown cecropin P1 peptide
SWLSKTAKKLENSAKKRISEGIAIAIQGGPR

SEQ ID NO: 47 - protein, 30 aa, Unknown
  Description of Unknown alpha-defensin peptide
ACYCRIPACIAGERRYGTCIYQGRLWAFCC

SEQ ID NO: 48 - protein, 36 aa, Unknown
  Description of Unknown beta-defensin peptide
DHYNCVSSGGQCLYSACPIFTKIQGTCYRGKAKCCK

SEQ ID NO: 49 - protein, 42 aa, Unknown
  Description of Unknown PR-39 peptide
RRRPRPPYLPRPRPPPFFPPRLPPRIPPGFPPRFPPRFPGKR

SEQ ID NO: 50 - protein, 13 aa, Unknown
  Description of Unknown indolicidin peptide
ILPWKWPWWPWRR

SEQ ID NO: 51 - protein, 16 aa, Unknown
  Description of Unknown RFGF peptide
AAVALLPAVLLALLAP

SEQ ID NO: 52 - protein, 11 aa, Unknown
  Description of Unknown RFGF analogue peptide
AALLPVLLAAP

SEQ ID NO: 53 - protein, 12 aa, Unknown
  Description of Unknown bactenecin peptide
RKCRIVVIRVCR

SEQ ID NO: 54 - DNA, 20 bp, Artificial Sequence
  Description of Artificial Sequence Synthetic primer
gctgtcacaaatggaggtgc

SEQ ID NO: 55 - DNA, 23 bp, Artificial Sequence
  Description of Artificial Sequence Synthetic primer
tcaagtggggcgatgctggcgct

SEQ ID NO: 56 - DNA, 22 bp, Artificial Sequence
  Description of Artificial Sequence Synthetic primer
agggggccctccgacgcctgct

SEQ ID NO: 57 - RNA, 23 nt, Artificial Sequence
  Description of Artificial Sequence Synthetic oligonucleotide
aaacucagaacuuuauccuuauu

SEQ ID NO: 58 - RNA, 19 nt, Artificial Sequence
  Description of Artificial Sequence Synthetic oligonucleotide
uuaccuuuggaaggagcua

SEQ ID NO: 59 - RNA, 21 nt, Artificial Sequence
  Description of Artificial Sequence Synthetic oligonucleotide
gggagaaagaggagcgugauu

SEQ ID NO: 60 - RNA, 19 nt, Artificial Sequence
  Description of Artificial Sequence Synthetic oligonucleotide
gguauaggauuuugaagca

SEQ ID NO: 61 - RNA, 18 nt, Artificial Sequence
  Description of Artificial Sequence Synthetic oligonucleotide
ggauaacgauggcuacua

SEQ ID NO: 62 - RNA, 19 nt, Artificial Sequence
  Description of Artificial Sequence Synthetic oligonucleotide
guaaugaucgaccgaaugu

SEQ ID NO: 63 - RNA, 19 nt, Artificial Sequence
  Description of Artificial Sequence Synthetic oligonucleotide
ccaccacguucaaugggaa

SEQ ID NO: 64 - RNA, 19 nt, Artificial Sequence
  Description of Artificial Sequence Synthetic oligonucleotide
uagcagaaauguacaacua

SEQ ID NO: 65 - RNA, 19 nt, Artificial Sequence
  Description of Artificial Sequence Synthetic oligonucleotide
gcgcaauaauguauacuuu

SEQ ID NO: 66 - DNA, 20 bp, Artificial Sequence
  Description of Artificial Sequence Synthetic primer
ttctaggagtgcggccaaag

SEQ ID NO: 67 - DNA, 20 bp, Artificial Sequence
  Description of Artificial Sequence Synthetic primer
cagctctccaccatctccac

SEQ ID NO: 68 - DNA, 20 bp, Artificial Sequence
  Description of Artificial Sequence Synthetic primer
gccccagttcagccaatact

SEQ ID NO: 69 - DNA, 20 bp, Artificial Sequence
  Description of Artificial Sequence Synthetic primer
accatacaaggccaccaagg

SEQ ID NO: 70 - DNA, 23 bp, Artificial Sequence
  Description of Artificial Sequence Synthetic primer
cctggaggagaagaggaaagaga

SEQ ID NO: 71 - DNA, 25 bp, Artificial Sequence
  Description of Artificial Sequence Synthetic primer
ttgaggacctctgtgtatttgtcaa

SEQ ID NO: 72 - DNA, 50 bp, Artificial Sequence
  Description of Artificial Sequence Synthetic oligonucleotide
ccaacgcagagtgtatcatctcctgaagcaacatctggttctacagcatc

SEQ ID NO: 73 - DNA, 50 bp, Artificial Sequence
  Description of Artificial Sequence Synthetic oligonucleotide
caacgcagagtgtaccatctcctgaagcaacatctggttctacagcatct

SEQ ID NO: 74 - DNA, 50 bp, Artificial Sequence
  Description of Artificial Sequence Synthetic oligonucleotide
caacgcagagtgtatcatctcctgaagcaacatctggttctacagcatct

SEQ ID NO: 75 - DNA, 50 bp, Artificial Sequence
  Description of Artificial Sequence Synthetic oligonucleotide
caacgcagagtgtatcatctcctgaagcaacatctggttctacagcgtct

SEQ ID NO: 76 - DNA, 50 bp, Artificial Sequence
  Description of Artificial Sequence Synthetic oligonucleotide
aacgcagagtgtatcatctcctgaagcaacatctggttctacagcatctg

SEQ ID NO: 77 - DNA, 50 bp, Artificial Sequence
  Description of Artificial Sequence Synthetic oligonucleotide
ctcgcagagtgtatcatctcctgaagcaacatctggttctacagcatctg

SEQ ID NO: 78 - DNA, 50 bp, Artificial Sequence
  Description of Artificial Sequence Synthetic oligonucleotide
acgcagagtgtatcatctcctgaagcaacatctggttctacagcatctgc

SEQ ID NO: 79 - DNA, 50 bp, Artificial Sequence
  Description of Artificial Sequence Synthetic oligonucleotide
cgcagagtgtattatctcctgaagcaacatctggttctacagcatctgca

SEQ ID NO: 80 - DNA, 50 bp, Artificial Sequence
  Description of Artificial Sequence Synthetic oligonucleotide
cgcagagtgtatcatctcctgaagcaacatctggttctacagcatctgag

SEQ ID NO: 81 - DNA, 50 bp, Artificial Sequence
  Description of Artificial Sequence Synthetic oligonucleotide
cgcagagtgtatcatctcctgaagcaacatctggttctacagcatctgca

SEQ ID NO: 82 - DNA, 50 bp, Artificial Sequence
  Description of Artificial Sequence Synthetic oligonucleotide
gcagagtgtatcatctcctgaagcaacatctggttctacagcatctgcat

SEQ ID NO: 83 - DNA, 50 bp, Artificial Sequence
  Description of Artificial Sequence Synthetic oligonucleotide
cagagtgtatcatctcctgaagcaacatctggttctacagcatctgcatt

SEQ ID NO: 84 - DNA, 50 bp, Artificial Sequence
  Description of Artificial Sequence Synthetic oligonucleotide
agagtgtatcatctcctgaagcaacatctggttctacagcatctgcattg

SEQ ID NO: 85 - DNA, 50 bp, Artificial Sequence
  Description of Artificial Sequence Synthetic oligonucleotide
tgagtgtatcatctcctgaagcaacatctggttctacagcatctgcattg

SEQ ID NO: 86 - DNA, 50 bp, Artificial Sequence
  Description of Artificial Sequence Synthetic oligonucleotide
gagtgtatcatctcctgaagcaacatctggttctacagcatctgcattgc

SEQ ID NO: 87 - DNA, 50 bp, Artificial Sequence
  Description of Artificial Sequence Synthetic oligonucleotide
agtgtatcatctcctgaagcaacatctggttctacagcatctgcattgcc

SEQ ID NO: 88 - DNA, 50 bp, Artificial Sequence
  Description of Artificial Sequence Synthetic oligonucleotide
gtgtatcatctcctgaagcaacatctggttctacagcatctgcattgccg

SEQ ID NO: 89 - DNA, 50 bp, Artificial Sequence
  Description of Artificial Sequence Synthetic oligonucleotide
tgtatcatctcctgaagcaacatctggttctacagcatctgcattgccgg

SEQ ID NO: 90 - DNA, 50 bp, Artificial Sequence
  Description of Artificial Sequence Synthetic oligonucleotide
tattatctcctgaagcaacatctggttctacagcatctgcattgccggga

SEQ ID NO: 91 - DNA, 50 bp, Artificial Sequence
  Description of Artificial Sequence Synthetic oligonucleotide
tatcatctcctgaagcaacatctggttctacagcatctgcattgccggga

SEQ ID NO: 92 - DNA, 50 bp, Artificial Sequence
  Description of Artificial Sequence Synthetic oligonucleotide
atcatctcctgaagcaacatctggttctacagcatctgcattgccgggac

SEQ ID NO: 93 - DNA, 50 bp, Artificial Sequence
  Description of Artificial Sequence Synthetic oligonucleotide
tcatctcctgaagcaacatctggttctacagcatctgcattgccgggacc

SEQ ID NO: 94 - DNA, 50 bp, Artificial Sequence
  Description of Artificial Sequence Synthetic oligonucleotide
catctcctgaagcaacatctggttctacagcatctgcattgccgggaccg

SEQ ID NO: 95 - DNA, 50 bp, Artificial Sequence
  Description of Artificial Sequence Synthetic oligonucleotide
atctcctgaagcaacatctggttctacagcatctgcattgccgggaccgg

SEQ ID NO: 96 - DNA, 50 bp, Artificial Sequence
  Description of Artificial Sequence Synthetic oligonucleotide
tctcctgaagcaacatctggttctacagcatctgcattgccgggaccgga

SEQ ID NO: 97 - DNA, 50 bp, Artificial Sequence
  Description of Artificial Sequence Synthetic oligonucleotide
ctcctgaagcaacatctggttctacagcatctgcattgccgggaccggat

SEQ ID NO: 98 - DNA, 50 bp, Artificial Sequence
  Description of Artificial Sequence Synthetic oligonucleotide
tcctgaagcaacatctggttctacagcatctgcattgccgggaccggata

SEQ ID NO: 99 - DNA, 50 bp, Artificial Sequence
  Description of Artificial Sequence Synthetic oligonucleotide
ctgaagcaacatctggttctacagcatctgcattgccgggaccggatatg

SEQ ID NO: 100 - DNA, 50 bp, Artificial Sequence
  Description of Artificial Sequence Synthetic oligonucleotide
tgaagcaacatctggttctacagcatctgcattgccgggaccggatatga

SEQ ID NO: 101 - DNA, 50 bp, Artificial Sequence
  Description of Artificial Sequence Synthetic oligonucleotide
gaagcaacatctggttctacagcatctgcattgccgggaccggatatgag

SEQ ID NO: 102 - DNA, 50 bp, Artificial Sequence
  Description of Artificial Sequence Synthetic oligonucleotide
aagcaacatctggttctacagcatctgcattgccgggaccggatatgagc

SEQ ID NO: 103 - DNA, 50 bp, Artificial Sequence
  Description of Artificial Sequence Synthetic oligonucleotide
agcaacatctggttctacagcatctgcattgccgggaccggatatgagca

SEQ ID NO: 104 - DNA, 50 bp, Artificial Sequence
  Description of Artificial Sequence Synthetic oligonucleotide
gcaacatctggttctacagcatctgcattgccgggaccggatatgagcat

SEQ ID NO: 105 - DNA, 50 bp, Artificial Sequence
  Description of Artificial Sequence Synthetic oligonucleotide
caacatctggttctacagcatctgcattgccgggaccggatatgagcatg

SEQ ID NO: 106 - DNA, 50 bp, Artificial Sequence
  Description of Artificial Sequence Synthetic oligonucleotide
aacatctggttctacagcatctgcattgccgggaccggatatgagcatga

SEQ ID NO: 107 - DNA, 50 bp, Artificial Sequence
  Description of Artificial Sequence Synthetic oligonucleotide
acatctggttctacagcatctgcattgccgggaccggatatgagcatgaa

SEQ ID NO: 108 - DNA, 50 bp, Artificial Sequence
  Description of Artificial Sequence Synthetic oligonucleotide
atctggttctacagcatctgcattgccgggaccggatatgagcatgaaac

SEQ ID NO: 109 - DNA, 50 bp, Artificial Sequence
  Description of Artificial Sequence Synthetic oligonucleotide
tctggttctacagcatctgcattgccgggaccggatatgagcatgaaacc

SEQ ID NO: 110 - DNA, 50 bp, Artificial Sequence
  Description of Artificial Sequence Synthetic oligonucleotide
ctggttctacagcatctgcattgccgggaccggatatgagcatgaaacct

SEQ ID NO: 111 - DNA, 50 bp, Artificial Sequence
  Description of Artificial Sequence Synthetic oligonucleotide
tggttctacagcatctgcattgccgggaccggatatgagcatgaaaccta

SEQ ID NO: 112 - DNA, 50 bp, Artificial Sequence
  Description of Artificial Sequence Synthetic oligonucleotide
ggttctacagcatctgcattgccgggaccggatatgagcatgaaacctag

SEQ ID NO: 113 - DNA, 50 bp, Artificial Sequence
  Description of Artificial Sequence Synthetic oligonucleotide
gttctacagcatctgcattgccgggaccggatatgagcatgaaacctagt